(19) United States
(12) Patent Application Publication
(10) Pub. No.: US 2024/0395384 A1
(43) Pub. Date: Nov. 28, 2024

(54) PATIENT CENTRIC PRECISION MODEL FOR ANTI-TNF THERAPY

(71) Applicant: Prometheus Laboratories Inc., San Diego, CA (US)

(72) Inventor: Thierry DERVIEUX, San Diego, CA (US)

(21) Appl. No.: 18/689,264

(22) PCT Filed: Sep. 29, 2022
(86) PCT No.: PCT/US2022/045261

§ 371 Date: Mar. 5, 2024

Related U.S. Application Data
(60) Provisional application No. 63/261,938, filed on Sep. 30, 2021.
(60) Provisional application No. 63/273,082, filed on Oct. 28, 2021.

(51) Int. Cl. G16H 20/17; A61K 31/4245; A61K 31/519; A61P 37/06; C07K 16/24; G16B 20/20; G16H 10/60; G16H 50/70

(52) U.S. Cl. CPC G16H 20/17; A61K 31/4245; A61K 31/519; A61P 37/06; C07K 16/241; G16B 20/20; G16H 10/60; G16H 50/70

(57) ABSTRACT

Provided are systems and methods for treating an immune-mediated inflammatory disease (e.g., inflammatory bowel disease) in a subject or selecting the subject for treatment, based on an estimated time to remission following induction of an anti-TNF therapy calculated by a patient-centric precision model.

### CROSS-REFERENCE

This application claims the benefit of U.S. Provisional Application No. 63/261,938, filed Sep. 30, 2021, and U.S. Provisional Application No. 63/273,082, filed Oct. 28, 2021, each of which is incorporated herein by reference in its entirety.

### BACKGROUND

Inflammatory bowel disease (IBD) is a chronic inflammation of tissues in the digestive track marked by cycles of active disease and remission. Types of IBD include ulcerative colitis (UC) and Crohn's disease (CD). Symptoms of IBD include diarrhea with or without blood, abdominal pain, weight loss, and fatigue. Noncurative therapies are widely used to relieve IBD patients of these symptoms, but they often result in patient relapse and treatment failures overtime. Targeted IBD treatment modalities encompass anti-tumor necrosis factor (TNF) agents. Anti-TNF agents include Infliximab (IFX), adalimumab (ADA), certolizumab pegol, and golimumab, all of which are monoclonal antibodies targeting TNF to induce and maintain remission in patients with IBD. However, one-fifth of IBD patients will have no response at all to these agents (primary non-response, PNR) and an additional one-third will eventually fail therapy (secondary loss of response), requiring an addition or change to another medication or surgery.

Population pharmacokinetic models are used to describe the time course of drug exposure in patients, and can be used to simulate alternative dose regimens to achieve a desired outcome.

### SUMMARY

The present disclosure provides one or more statistical models that predicts whether a patient will fail therapeutically respond to an induction of a biologic drug (e.g., an anti-TNF agent), or lose response to the biologic drug overtime. Also provided are methods of utilizing the one or more statistical modules disclosed herein to optimize a treatment course for the patient, which may include modulating the dose, inter-dose interval, or type of therapeutic agent to achieve and sustain disease remission, based at least in part on the output from the one or more statistical models. The one or more statistical models disclosed herein predict disease remission using a combination of different parameters, such as, for example, (i) estimated clearance of the therapeutic agent (e.g., using weight, albumin, and/or c-reactive protein (CRP) as covariates), (ii) presence of risk genotypes associated with increased risk for developing severe disease and autoantibodies against the biologic drug, (iii) a presence or high level of serological markers, (iv) patient wellbeing information from the subject, such as symptoms, disease activity scores, and the like, or (v) any combination thereof.

Aspects disclosed herein provide methods for treating an immune-mediated inflammatory disease in a subject, said method comprising: administering a dose of an anti-tumor necrosis factor (TNF) therapy to the subject to treat the immune-mediated inflammatory disease, wherein the subject is selected based on a combined poor prognosis factor (PPF) score below 3 on a scale of 0 to 3 calculated using a statistical model, wherein the combined PPF score reflects a clearance estimate of the anti-TNF therapy and a presence of a genotype at a single nucleotide variant (SNV) comprising rs2097432 or a SNV in linkage disequilibrium therewith detected in a biological sample obtained from the subject. In some embodiments, the anti-TNF therapy comprises infliximab, adalimumab, etanercept, golimumab, or certolizumab. In some embodiments, the statistical model comprises one or more algorithms comprising a Naïve Bayes classifier algorithm, a non-linear mixed effects model (NLME), or a Metropolis Hastings algorithm, or any combination thereof. In some embodiments, the statistical model predicts a time to remission for the subject of the immune-mediated inflammatory disease. In some embodiments, the statistical model predicts the time to remission for the subject by a method comprising: (a) receiving data from a database, wherein the data is related to disease remission in individuals from a reference population having the immune-mediated inflammatory disease that have been treated with the anti-TNF therapy; (b) establishing a first set of parameter estimates from the data; (c) deriving a second set of parameter estimates for the statistical model based at least in part on the first set of parameter estimates; (d) receiving subject specific data comprising: (i) the genotype at the SNV comprising rs2097432 or the SNV in linkage disequilibrium therewith; (ii) a weight or body mass index (BMI) of the subject; and (iii) a level of albumin measured in the biological sample; (e) updating the model based at least in part on the subject specific data received in (d); and (f) determining the time to remission for the subject, wherein the time to remission is reflected in a combined PPF score on the scale of 0 to 3. In some embodiments, the subject specific data further comprises a level of c-reactive protein (CRP), wherein a CRP above 3 milligrams per liter (mg/L) is indicative of a presence of inflammation in the subject. In some embodiments, the subject specific data further comprises a level of autoantibodies against the anti-TNF therapy, a level of the anti-TNF therapy, or a combination thereof. In some embodiments, the combined PPF score further reflects a wellbeing of the subject based on patient wellbeing data received by the structural model. In some embodiments, the patient wellbeing data comprises a score on a disease activity index for the immune-mediated inflammatory disease. In some embodiments, the disease activity index is Crohn's Disease Activity Index (CDAI) when the immune-mediated inflammatory disease is Crohn's disease. In some embodiments, the disease activity index is a Mayo Clinical Score when the immune-mediated inflammatory disease is Ulcerative Colitis. In some embodiments, the patient wellbeing data comprises symptoms of the immune-mediated inflammatory disease, wherein the symptoms comprise diarrhea, abdominal pain, abdominal cramping, loss of appetite, weight loss, fatigue, fever, rectal bleeding, a skin rash, joint pain, stiffness, or swelling, decreased range of motion for joints, redness at joints, or any combination thereof. In some embodiments, the patient wellbeing data comprises body mass index (BMI) or weight.

Aspects disclosed herein provide methods for treating an immune-mediated inflammatory disease in a subject, said method comprising: administering a dose of an immune-mediated inflammatory disease therapy other than an anti-TNF therapy to the subject to treat the immune-mediated inflammatory disease, wherein the subject was selected based on combined poor prognosis factor (PPF) score of 3 on a scale of 0 to 3 calculated using a statistical model, wherein the combined PPF score reflects a clearance estimate of the anti-TNF therapy and a presence of a genotype at a single nucleotide variant (SNV) comprising rs2097432 or a SNV in linkage disequilibrium therewith detected in a biological sample obtained from the subject. In some embodiments, the immune-mediated inflammatory disease therapy other than the anti-TNF therapy comprises a small molecule Janus Kinase (JAK) or a small molecule modulator of sphingosine 1-phosphate (S1P). In some embodiments, the anti-TNF therapy comprises infliximab, adalimumab, etanercept, golimumab, or certolizumab. In some embodiments, the statistical model comprises one or more algorithms comprising a Naïve Bayes classifier algorithm, a non-linear mixed effects model (NLME), or a Metropolis Hastings algorithm, or any combination thereof. In some embodiments, the statistical model predicts a time to remission for the subject of the immune-mediated inflammatory disease. In some embodiments, the statistical model predicts the time to remission for the subject by a method comprising: (a) receiving data from a database, wherein the data is related to disease remission in individuals from a reference population having the immune-mediated inflammatory disease that have been treated with the anti-TNF therapy; (b) establishing a first set of parameter estimates from the data; (c) deriving a second set of parameter estimates for the statistical model based at least in part on the first set of parameter estimates; (d) receiving subject specific data comprising: (i) the genotype at the SNV comprising rs2097432 or the SNV in linkage disequilibrium therewith; (ii) a weight or body mass index (BMI) of the subject; and (iii) a level of albumin measured in the biological sample; (e) updating the model based at least in part on the subject specific data received in (d); and (f) determining the time to remission for the subject, wherein the time to remission is reflected in a combined PPF score on the scale of 0 to 3. In some embodiments, the subject specific data further comprises a level of c-reactive protein (CRP), wherein a CRP above 3 milligrams per liter (mg/L) is indicative of a presence of inflammation in the subject. In some embodiments, the subject specific data further comprises a level of autoantibodies against the anti-TNF therapy, a level of the anti-TNF therapy, or a combination thereof. In some embodiments, the combined PPF score further reflects a wellbeing of the subject based on patient wellbeing data received by the structural model. In some embodiments, the patient wellbeing data comprises a score on a disease activity index for the immune-mediated inflammatory disease. In some embodiments, the disease activity index is Crohn's Disease Activity Index (CDAI) when the immune-mediated inflammatory disease is Crohn's disease. In some embodiments, the disease activity index is a Mayo Clinical Score when the immune-mediated inflammatory disease is Ulcerative Colitis. In some embodiments, the patient wellbeing data comprises symptoms of the immune-mediated inflammatory disease, wherein the symptoms comprise diarrhea, abdominal pain, abdominal cramping, loss of appetite, weight loss, fatigue, fever, rectal bleeding, a skin rash, joint pain, stiffness, or swelling, decreased range of motion for joints, redness at joints, or any combination thereof. In some embodiments, the patient wellbeing data comprises body mass index (BMI) or weight.

Aspects disclosed herein provide methods for treating an immune-mediated inflammatory disease in a subject, said method comprising: (a) calculating a poor prognosis factor 1 (PPF1) score for the subject comprising: (i) obtaining or having obtained a biological sample from the subject; (ii) measuring a level of albumin in the biological sample; (iii) calculating a rate of which an anti-tumor necrosis factor (TNF) therapy is estimated to be cleared from the subject using a first statistical model based, at least in part, on the level of the albumin measured in the biological sample and a weight of the subject; and (iv) comparing the rate to a predetermined threshold for the anti-TNF therapy, to produce an estimated clearance score on a scale of 0 to 1, wherein 0 is indicative of a low rate of clearance below the predetermined threshold, and 1 is indicative of a high rate of clearance above the predetermined threshold; (b) calculating a poor prognosis factor 2 (PPF 2) score for the subject, comprising: (i) obtaining or having obtained the biological sample from the subject; (ii) performing or having performed a genotyping assay on the biological sample to detect a genotype at single nucleotide variant (SNV) comprising rs2097432 or a SNV in linkage disequilibrium therewith; and (iii) calculating a PPF2 score using a second statistical model, wherein the PPF2 score is on a scale of 0 to 2, wherein 0 is indicative of the genotype that is homozygous non-risk, 1 is indicative of the genotype that is heterozygous risk at rs2097432, and 2 comprises a presence of two genetic loci of said genotype; (c) calculating a poor prognosis factor 4 (PPF 4) for the subject comprising: (1) receiving wellbeing data from said subject comprising one or more symptoms related to the immune-mediated inflammatory disease; (ii) calculating a wellbeing score with a fourth statistical model of 0 or 1 based on the wellbeing data received, wherein 0 is indicative of an improvement of at least one symptom of the one or more symptoms, and 1 is indicative of no improvement in the at least one symptom of the one or more symptoms; and (d) calculating a combined PPF score comprising the PPF1 score, the PPF2 score, and the PPF4 score to predict a time to remission following administration of the anti-TNF therapy to the subject; (e) if the time to remission predicted in (d) for the subject is above a pre-specified threshold, then administering a first dosage of the anti-TNF therapy to the subject to treat the immune-mediated inflammatory disease in the subject; and (f) if the time to remission predicted in (d) for the subject is below the pre-specified threshold, then administering to the subject: a second dosage of the anti-TNF therapy, wherein the first dosage of the anti-TNF therapy is different than the second dosage of the anti-TNF therapy; or (ii) a therapy other than the anti-TNF therapy. In some embodiments, the methods further comprise calculating a poor prognosis factor 3 (PPF 3) score for the subject, comprising: (i) obtaining or having obtained the biological sample from the subject; (ii) performing or having performed an immunological assay on the biological sample to detect a level of a serological marker; and (iii) calculating a PPF3 score using a third statistical model, wherein the PPF3 score is on a scale of 0 to 2, wherein 0 is indicative of a standard response to the anti-TNF therapy, 1 is indicative e of a low response to the anti-TNF therapy, and 2 is indicative of very low response to the anti-TNF therapy. In some embodiments, the serological marker comprises perinuclear antineutrophil cytoplasmic antibodies (pANCA), proteinase 3 (PR3), or one or more antibodies against an integrin. In some embodiments, the predetermined threshold comprises about 0.2 to 0.4 L/day of the anti-TNF therapy. In some embodiments, the predetermined threshold is above about 0.3 L/day of the anti-TNF therapy. In some embodiments, the anti-TNF therapy is Infliximab. In some embodiments, the pre-specified threshold is a percentage reduction in hazard remission above 78%. In some embodiments, the first dose of the anti-TNF therapy comprises 5 milligrams per kilogram (mg/Kg) of Infliximab. In some embodiments, the pre-specified threshold is a percentage reduction in hazard remission comprising from 56% to 78%. In some embodiments, the first dose of the anti-TNF therapy comprises 7.5 mg/Kg of Infliximab. In some embodiments, the pre-specified threshold is a percentage reduction in hazard remission that comprises from 34 to 56%. In some embodiments, the first dose of the anti-TNF therapy comprises 10 mg/Kg of Infliximab. In some embodiments, the pre-specified threshold is a percentage reduction in hazard remission comprising 34% or less. In some embodiments, the therapy other than the anti-TNF therapy comprises a small molecule Janus Kinase (JAK) or a small molecule modulator of sphingosine 1-phosphate (S1P). In some embodiments, the small molecule inhibitor of JAK comprises Tofacitinib. In some embodiments, the small molecule modulator of S1P comprises Ozanimod. In some embodiments, the first statistical model, the second statistical model, the third statistical model, or the fourth statistical model comprises one or more algorithms comprising a Naïve Bayes classifier algorithm, a non-linear mixed effects model (NLME), or a Metropolis Hastings algorithm, or any combination thereof. In some embodiments, the anti-TNF therapy comprises Infliximab, adalimumab, etanercept, golimumab, or certolizumab. In some embodiments, the immune-mediated inflammatory disease comprises an inflammatory bowel disease (IBD), rheumatoid arthritis (RA), cytokine release syndrome, multiple sclerosis (MS), ankylosing spondylitis (AS), lupus, plaque psoriasis, atopic dermatitis, gout, migraine, cancer, or a neoplasm. In some embodiments, the IBD comprises Crohn's disease. In some embodiments, the IBD comprises Ulcerative Colitis. In some embodiments, the biological sample obtained from the subject is blood serum. In some embodiments, the genotype at the SNV comprising rs2097432 comprises a “G.” In some embodiments, the genotype at the SNV comprising s2097432 is homozygous. In some embodiments, the linkage disequilibrium is determined with an r² of at least about 0.84. In some embodiments, the subject is age 18 or older.

Aspects disclosed herein provide computer-implemented systems comprising a computing device comprising at least one processor, an operating system configured to perform executable instructions, a memory, and a computer program including instructions executable by the computing device to perform a method comprising: (a) calculating a poor prognosis factor 1 (PPF1) score for the subject comprising: (i) receiving a level of albumin measured in a biological sample obtained from a subject receiving anti-tumor necrosis factor (TNF) therapy for treatment of a immune-mediated inflammatory disease, and a weight of the subject; (ii) calculating a rate of which the anti-TNF therapy is estimated to be cleared from the subject using a first statistical model based, at least in part, on the level of the albumin measured in the biological sample, and the weight of the subject; and (iii) comparing the rate to a predetermined threshold for the anti-TNF therapy, to produce an estimated clearance score on a scale of 0 to 1, wherein 0 is indicative of a low rate of clearance below the predetermined threshold, and 1 is indicative of a high rate of clearance above the predetermined threshold; (b) calculating a poor prognosis factor 2 (PPF 2) score for the subject comprising: (i) receiving a genotype of the subject at a single nucleotide variant (SNV) comprising rs2097432 or a SNV in linkage disequilibrium therewith; and (ii) calculating a PPF2 score using a second statistical model, wherein the PPF2 score is on a scale of 0 to 2, wherein 0 is indicative of the genotype that is homozygous non-risk, 1 is indicative of the genotype that is heterozygous risk at rs2097432, and 2 comprises a presence of two genetic loci of said genotype; and (c) calculating a combined PPF score comprising the PPF1 score and the PPF2 score, to predict a time to remission following administration of the anti-TNF therapy to the subject. In some embodiments, the method further comprises: (d) calculating a poor prognosis factor 3 (PPF 3) score for the subject, comprising: (i) receiving a level of a serological marker detected in the biological sample obtained from the subject; and (ii) calculating a PPF3 score using a third statistical model, wherein the PPF3 score is on a scale of 0 to 2, wherein 0 is indicative of a standard response to the anti-TNF therapy, 1 is indicative e of a low response to the anti-TNF therapy, and 2 is indicative of very low response to the anti-TNF therapy. In some embodiments, the serological marker comprises perinuclear antineutrophil cytoplasmic antibodies (pANCA), proteinase 3 (PR3), or one or more antibodies against an integrin. In some embodiments, the method further comprises: (e) calculating a poor prognosis factor 4 (PPF 4) for the subject comprising: (i) receiving wellbeing data from the subject comprising one or more symptoms related to the immune-mediated inflammatory disease; (ii) calculating a wellbeing score with a fourth statistical model of 0 or 1 based on the wellbeing data received, wherein 0 is indicative of an improvement of at least one symptom of the one or more symptoms, and 1 is indicative of no improvement in the at least one symptom of the one or more symptoms, wherein the combined PPF score calculated in (c) further comprises the PPF score calculated in (d). In some embodiments, the anti-TNF therapy comprises Infliximab, Adalimumab, Etanercept, Golimumab, or Certolizumab. In some embodiments, the immune-mediated inflammatory disease comprises an inflammatory bowel disease (IBD), rheumatoid arthritis (RA), cytokine release syndrome, multiple sclerosis (MS), ankylosing spondylitis (AS), lupus, plaque psoriasis, atopic dermatitis, gout, migraine, cancer, or a neoplasm. In some embodiments, the IBD comprises Crohn's disease. In some embodiments, the IBD comprises Ulcerative Colitis. In some embodiments, the systems further comprise the biological sample, wherein the biological sample is blood serum. In some embodiments, the genotype at the SNV comprising rs2097432 comprises a “G.” In some embodiments, the genotype at the SNV comprising s2097432 is homozygous. In some embodiments, the linkage disequilibrium is determined with an r² of at least about 0.84. In some embodiments, the subject is age 18 or older. In some embodiments, the predetermined threshold comprises about 0.2 to 0.4 L/day of the anti-TNF therapy. In some embodiments, the predetermined threshold is above about 0.3 L/day of the anti-TNF therapy. In some embodiments, the method further comprising determining a percentage reduction in hazard remission based on the time to remission. In some embodiments, the percentage reduction in hazard remission is above 34%, then the subject is indicated for treatment with Infliximab. In some embodiments, if the percentage reduction in hazard remission is above 78%, then the subject is indicated for treatment with 5 mg/Kg of Infliximab. In some embodiments, if the percentage reduction in hazard remission comprises from 56% to 78%, then the subject is indicated for treatment with 7.5 mg/Kg of Infliximab. In some embodiments, if the percentage reduction in hazard remission comprises from 34% to 56%, then the subject is indicated for treatment with 10 mg/Kg of Infliximab. In some embodiments, if the percentage reduction in hazard remission is 34% or below, then the subject is indicated for treatment with a therapy other than the anti-TNF therapy.

Aspects disclosed herein provide non-transitory computer readable storage media encoded with a computer program including instructions executable by one or more processors to statistic model for predicting disease remission in a subject with an immune-mediated inflammatory disease, the computer program configured to cause the one or more processors to perform a method comprising: (a) calculating a poor prognosis factor 1 (PPF1) score for the subject comprising: (i) receiving a level of albumin measured in a biological sample obtained from a subject receiving anti-tumor necrosis factor (TNF) therapy for treatment of a immune-mediated inflammatory disease, and a weight of the subject; (ii) calculating a rate of which the anti-TNF therapy is estimated to be cleared from the subject using a first statistical model based, at least in part, on the level of the albumin measured in the biological sample, and the weight of the subject; and (iii) comparing the rate to a predetermined threshold for the anti-TNF therapy, to produce an estimated clearance score on a scale of 0 to 1, wherein 0 is indicative of a low rate of clearance below the predetermined threshold, and 1 is indicative of a high rate of clearance above the predetermined threshold; (b) calculating a poor prognosis factor 2 (PPF 2) score for the subject comprising: (i) receiving a genotype of the subject at a single nucleotide variant (SNV) comprising rs2097432 or a SNV in linkage disequilibrium therewith; and (ii) calculating a PPF2 score using a second statistical model, wherein the PPF2 score is on a scale of 0 to 2, wherein 0 is indicative of the genotype that is homozygous non-risk, 1 is indicative of the genotype that is heterozygous risk at rs2097432, and 2 comprises a presence of two genetic loci of said genotype; and (iii) calculating a combined PPF score comprising the PPF1 score, the PPF2 score, and the PPF3 score to predict a time to remission following administration of the anti-TNF therapy to the subject. In some embodiments, the method further comprises: (d) calculating a poor prognosis factor 3 (PPF 3) score for the subject, comprising: (i) receiving a level of a serological marker detected in the biological sample obtained from the subject; and (ii) calculating a PPF3 score using a third statistical model, wherein the PPF3 score is on a scale of 0 to 2, wherein 0 is indicative of a standard response to the anti-TNF therapy, 1 is indicative e of a low response to the anti-TNF therapy, and 2 is indicative of very low response to the anti-TNF therapy. In some embodiments, the serological marker comprises perinuclear antineutrophil cytoplasmic antibodies (pANCA), proteinase 3 (PR3), or one or more antibodies against an integrin. In some embodiments, the method further comprises: (e) calculating a poor prognosis factor 4 (PPF 4) for the subject comprising: (i) receiving wellbeing data from the subject comprising one or more symptoms related to the immune-mediated inflammatory disease; (ii) calculating a wellbeing score with a fourth statistical model of 0 or 1 based on the wellbeing data received, wherein 0 is indicative of an improvement of at least one symptom of the one or more symptoms, and 1 is indicative of no improvement in the at least one symptom of the one or more symptoms, wherein the combined PPF score calculated in (c) further comprises the PPF score calculated in (d). In some embodiments, the anti-TNF therapy comprises Infliximab, adalimumab, etanercept, golimumab, or certolizumab. Th In some embodiments, the immune-mediated inflammatory disease comprises an inflammatory bowel disease (IBD), rheumatoid arthritis (RA), cytokine release syndrome, multiple sclerosis (MS), ankylosing spondylitis (AS), lupus, plaque psoriasis, atopic dermatitis, gout, migraine, cancer, or a neoplasm. In some embodiments, the IBD comprises Crohn's disease. In some embodiments, the IBD comprises Ulcerative Colitis. In some embodiments, the biological sample is blood serum. In some embodiments, the genotype at the SNV comprising rs2097432 comprises a “G.” In some embodiments, the genotype at the SNV comprising s2097432 is homozygous. In some embodiments, the linkage disequilibrium is determined with an r² of at least about 0.84. In some embodiments, the subject is age 18 or older. In some embodiments, the predetermined threshold comprises about 0.2 to 0.4 L/day of the anti-TNF therapy. In some embodiments, the predetermined threshold is above about 0.3 L/day of the anti-TNF therapy. In some embodiments, the method further comprising determining a percentage reduction in hazard remission based on the time to remission. In some embodiments, if the percentage reduction in hazard remission is above 34%, then the subject is indicated for treatment with Infliximab. In some embodiments, if the percentage reduction in hazard remission is above 78%, then the subject is indicated for treatment with 5 mg/Kg of Infliximab. In some embodiments, if the percentage reduction in hazard remission comprises from 56% to 78%, then the subject is indicated for treatment with 7.5 mg/Kg of Infliximab. In some embodiments, if the percentage reduction in hazard remission comprises from 34% to 56%, then the subject is indicated for treatment with 10 mg/Kg of Infliximab. In some embodiments, if the percentage reduction in hazard remission is 34% or below, then the subject is indicated for treatment with a therapy other than the anti-TNF therapy.

Aspects disclosed herein provide methods of predicting whether an immune-mediated inflammatory disease in a subject will positively respond to a treatment with an anti-TNF therapy, the method comprising: (a) calculating an estimated clearance rate at which an anti-tumor necrosis factor (TNF) therapy is cleared from the subject using a first statistical model based, at least in part, on a level of albumin measured in a biological sample obtained from the subject, and a weight of the subject; (b)

comparing the clearance rate calculated in (a) to a predetermined threshold, to produce poor prognosis factor 1 (PPF1) score on a scale of 0 to 1, wherein 0 is indicative of a low rate of clearance below the predetermined threshold, and 1 is indicative of a high rate of clearance above the predetermined threshold; (c) calculating a poor prognosis factor 2 (PPF2) score on a scale of 0 to 2 with a second statistical model, based at least in part, on a presence of a genotype at a single nucleotide variant (SNV) comprising rs2097432 or a SNV in linkage disequilibrium therewith detected in the biological sample obtained from the subject, wherein 0 is indicative of the genotype that is homozygous non-risk, 1 is indicative of the genotype that is heterozygous risk at rs2097432, and 2 comprises a presence of two genetic loci of said genotype; and (d) calculating a combined PPF score comprising the PPF1 score and the PPF2 score to predict whether the immune-mediated inflammatory disease in the subject will positively respond to the treatment with the anti-TNF therapy. In some embodiments, the methods further comprise (e) calculating a poor prognosis factor 4 (PPF4) score on a scale of 0 to 1 with a third statistical model, based at least in part on patient wellbeing received from the subject, wherein a PPF4 score of 0 is indicative of an improvement of at least one symptom of the immune-mediated inflammatory disease, and a PPF4 score of 1 is indicative of no improvement in the at least one symptom, and wherein the combined PPF score calculated in (d) further comprises the PPF4 score. In some embodiments, the methods further comprise (f) calculating a poor prognosis factor 3 (PPF3) score on a scale of 0 to 2 with a fourth statistic model, wherein 0 is indicative of a standard response to the anti-TNF therapy, 1 is indicative e of a low response to the anti-TNF therapy, and 2 is indicative of very low response to the anti-TNF therapy. In some embodiments, the predetermined threshold is about 0.2 to 0.4 L/day. In some embodiments, the predetermined threshold is above 0.3 L/day. In some embodiments, the methods further comprise calculating a time to remission for the subject, wherein the time to remission is expressed as a percentage reduction in hazard remission. In some embodiments, the percentage reduction in hazard remission is above 34% and the anti-TNF therapy comprises Infliximab. In some embodiments, the methods further comprise administering to the subject 5 mg/Kg of Infliximab, provided the percentage reduction in hazard remission is above 78%. In some embodiments, the methods further comprise administering to the subject 7.5 mg/Kg of Infliximab, provided the percentage reduction in hazard remission comprises from 56% to 78%.

In some embodiments, the methods further comprise administering to the subject 10 mg/Kg of Infliximab, provided the percentage reduction in hazard remission comprises 34% to 56%.

In some embodiments, the methods further comprise administering to the subject a therapy other than the anti-TNF therapy, provided the percentage reduction in hazard remission is 34% or below. In some embodiments, the therapy other than the anti-TNF therapy comprises a small molecule inhibitor of Janus Kinase (JAK) or a small molecule modulator of sphingosine 1-phosphate (S1P). In some embodiments, the small molecule inhibitor of Janus Kinase (JAK) comprises Tofacitinib. In some embodiments, the first statistical model and the second statistical model comprises one or more algorithms comprising a Naïve Bayes classifier algorithm, a non-linear mixed effects model (NLME), or a Metropolis Hastings algorithm, or any combination thereof. In some embodiments, the anti-TNF therapy comprises Infliximab, adalimumab, etanercept, golimumab, or certolizumab. In some embodiments, the immune-mediated inflammatory disease comprises an inflammatory bowel disease (IBD), rheumatoid arthritis (RA), cytokine release syndrome, multiple sclerosis (MS), ankylosing spondylitis (AS), lupus, plaque psoriasis, atopic dermatitis, gout, migraine, cancer, or a neoplasm. In some embodiments, the IBD comprises Crohn's disease. In some embodiments, the IBD comprises Ulcerative Colitis. In some embodiments, the biological sample obtained from the subject is blood serum. In some embodiments, the genotype at the SNV comprising rs2097432 comprises a “G.” In some embodiments, the genotype at the SNV comprising s2097432 is homozygous. In some embodiments, the linkage disequilibrium is determined with an r² of at least about 0.84. In some embodiments, the subject is age 18 or older. In some embodiments, the patient wellbeing data comprises a score on a disease activity index for the immune-mediated inflammatory disease. In some embodiments, the disease activity score index comprises Crohn's Disease Activity Index (CDAI) or a Mayo Clinical Score.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the inventive concepts are set forth with particularity in the appended claims. A better understanding of the features and advantages of the present inventive concepts will be obtained by reference to the following detailed description that sets forth illustrative embodiments, in which the principles of the inventive concepts are utilized, and the accompanying drawings of which:

FIG. 1 shows a diagram of the patient-centric precision model having four modules termed “poor prognosis factors (PPF),” which includes poor prognosis factor 1 (PPF1, “pharmacokinetic system”) to predict clearance of a biologic drug, poor prognosis factor 2 (PPF 2, “genetics system”) using genotypes at the HLADQA1*05 locus associated with increased risk for autoantibodies against the biologic drug, poor prognosis factor 3 (PPF3, “antigenic load”) to predict response to the biologic drug by measuring an inflammatory load using levels of biomarkers such as c-reactive protein (CRP), and/or perinuclear antineutrophil cytoplasmic antibodies (pANCA), proteinase 3 (PR3), or integrins, and poor prognosis factor 4 (PPF4, “patient wellbeing”), which includes disease activity scores or symptoms of disease monitored in real-time by the patient;

FIG. 2 shows a graph of baseline clearance in subjects predicted using the pharmacokinetic system (PPF1) in subjects with inflammatory bowel disease (IBD) using albumin and weight as covariates, at the point of the encounter, such as, for example when the patient visits the clinician office;

FIG. 3 shows a graph of time to remission in subjects based on baseline clearance;

FIG. 4 shows a non-limiting example of a computer interface for calculating baseline clearance in subjects;

FIG. 5 shows a graph of time to remission in subjects using the genetic system (PPF2), which is based on a number of alleles at the HLADQA1*05 locus in subjects with IBD, with #hla_nb_:0-1 referring to genotypes that are homozygous non-risk (score: 0), or heterozygous risk (score: 1), and #hla_nb_:1-2 referring to heterozygous risk (score: 1) or homozygous risk (score=2);

FIG. 6 shows a graph of time to remission based on the combined poor prognosis factor scoring system including PPF1 and PPF2;

FIG. 7 shows a graph of individualized times to remission in subjects; x axis is the score of a combined PPF including PPF1 and PPF2, and they axis is the time to remission in days;

FIG. 8 shows a non-limiting example of a mobile App interface for subjects to report patient wellbeing data for PPF4, which can include disease activity scores or symptom information related to the disease;

FIG. 9 shows a computer system of the present disclosure;

FIG. 10 shows a graph of time to remission in subjects;

FIG. 11 shows a graph of time to remission in subjects based on the number of alleles at the HLADQA1*05 locus in subjects;

FIG. 12 shows a graph of time to remission in subjects based on baseline clearance with #hla_nb_01:0 referring to genotypes that are homozygous non-risk (score: 0), and #hla_nb_01:1 referring to heterozygous risk (score: 1);

FIG. 13 shows a graph of time to remission based on the combined poor prognosis factor scoring system including PPF1 and PPF2;

FIG. 14 shows a graph of time to remission based on proactive maintenance of Infliximab (IFX) levels; #IItrough_trough:16.4372-56.2095 and #IItrough_trough: 0.667039-16.437 refers to a the recited ranges in trough concentration (mg/L);

FIG. 15 shows a graph of time to remission based on the combined poor prognosis factor scoring system including pharmacokinetic system (PF1) with IFX levels and genetic system (PF2);

FIG. 16 shows a graph of time to remission estimates based on the combined poor prognosis factor scoring system including pharmacokinetic system (PF1) with IFX levels and genetic system (PF2);

FIG. 17 shows a diagram of poor prognosis factors;

FIG. 18 shows graphs of time to remission analysis with induction estimates;

FIG. 19 show graphs of time to remission analysis with induction estimates;

FIG. 20 shows the time to remission analysis with induction estimates based on the combined poor prognosis pharmacokinetic factors for week 2, 6, and 14;

FIG. 21 shows estimated clearance of anti-TNF therapies, IFX and Adalimumab (ADA) using the poor prognosis factor 1 (PPF1) with and without c-reactive protein (CRP);

FIG. 22 shows the probability for sustained efficacy during treatment calculated using logistic regression;

FIG. 23 shows the sensitivity, specificity, positive predictive value and negative predictive value of PPF1;

FIG. 24 shows the probability to achieve disease control in the presence of clearance before starting therapy;

FIG. 25 shows the probability of sustained versus not sustained CRP-based clinical remission for IFX and ADA;

FIG. 26 shows validation in two independent cohorts of IBD patients of PPF1 for ADA without CRP;

FIG. 27 shows validation in two independent cohorts of IBD patients of PPF1 for ADA with CRP;

FIG. 28 shows a distribution of PPF1 scores across treatments;

FIG. 29 shows the median interquartile range of baseline clearance (L/day) in relation to ADA and IFX efficacy in inflammatory bowel disease patients with PPF, using weight, albumin and CRP as covariates;

FIG. 30 shows that patients with accelerated clearance have lower likelihood to achieve disease control following IFX or ADA;

FIG. 31 shows sustained remission over maintenance period with ADA starting 14 weeks after induction treatment with ADA;

FIG. 32 shows the positive and negative predictive value of accelerated clearance at various pre-test probabilities;

FIG. 33 shows a non-limiting example of a computing device; in this case, a device with one or more processors, memory, storage, and a network interface;

FIG. 34 shows a non-limiting example of a web/mobile application provision system; in this case, a system providing browser-based and/or native mobile user interfaces;

FIG. 35 shows a non-limiting example of a cloud-based web/mobile application provision system; in this case, a system comprising an elastically load balanced, auto-scaling web server and application server resources as well synchronously replicated databases;

FIG. 36 shows the distribution of scores associated with anti-TNF response, where the points are illustrated on the x axis and the frequency of subjects is shown on the y axis;

FIG. 37 shows the performance of the patient-centric precision model in predicting anti-TNF response based on the PPF1 scores (0, 1, 2) calculated;

FIG. 38 shows that sustained clinical remission in achieved in IBD patients having a PPF1 score of 2 (left) and the distribution of scores using the patient-centric precision model (right); and

FIG. 39 shows that a PPF3 score of 1 or 0 is predictive of sustained disease remission, as compared with a PPF3 score of 2.

### DETAILED DESCRIPTION

Provided herein are systems and methods for determining a dosage or a type of a therapeutic agent for treatment of an immune-mediated inflammatory disease (e.g., inflammatory bowel disease) in a subject using a patient-centric precision model for predicting a time to remission following induction of the therapeutic agent. In some embodiments, the therapeutic agent comprises an anti-Tumor Necrosis Factor (TNF) therapy. In some embodiments, the anti-TNF therapy comprises Remicade® (Infliximab), Enbrel® (Etanercept), Humira® (Adalimumab), Cimzia® (Certolizumab pegol), or Simponi® (Golimumab). The patient-centric precision model, in some embodiments, takes into consideration “poor prognosis factors (PPF)” such as the pharmacokinetic (PK) profile of the drug (e.g., estimated clearance time of the drug in the subject) (PPF1), the genetic predisposition of the subject to exhibit a longer time to remission following induction of the drug (PPF2), the presence or levels of serological markers (e.g., pANCA, PR3) measured in a biological sample obtained from the subject (PPF3), and/or patient-specific information regarding symptoms of the immune-mediated inflammatory disease (e.g., rectal bleeding, bloating, discomfort, diarrhea, constipation, and the like) (PPF4). In some embodiments, the patient-centric precision model calculates a combined PPF score to predict a time to remission following an induction of the biologic drug. The methods and systems described herein involve, in some embodiments, administering a certain dosage or type of the therapeutic agent disclosed herein (e.g., anti-TNF therapy) based, at least in part, on the combined PPF score of the subject.

Also provide are methods of utilizing the patient-centric precision model to predict whether treatment of an immune-mediated inflammatory disease of a subject disclosed herein with a biologic drug (e.g., an anti-TNF therapy) will result in disease remission (e.g., predict response v. non-response, or loss-of-response). If disease remission is predicted, then the subject may be indicated for treatment with the biologic drug. In some embodiments, the patient-centric precision model further predicts a particular dose or inter-dose interval for the biologic drug, or a type of biologic drug that is predicted to achieve and sustain disease remission. If disease remission is not predicted, then the subject may be indicated for a therapy other than the biologic drug. In some embodiments, the biologic drug comprises an anti-TNF therapy, an inhibitor α₄β₇ integrin, an inhibitor of interleukin 12 (IL-12), an inhibitor of interleukin 23 (IL-23), or any combination thereof. In some embodiments, the anti-TNF therapy comprises infliximab, adalimumab, etanercept, golimumab, or certolizumab. In some embodiments, the inhibitor of α₄β₇ integrin is or comprises vedolizumab (VDZ). In some embodiments, the inhibitor of IL-12 or IL-13, or a combination thereof, is or comprises ustekinumab (UST). In some embodiments, the therapy other than the biologic drug is or comprises a small molecule Janus Kinase (JAK) or a small molecule modulator of sphingosine 1-phosphate (S1P). In some embodiments, the small molecule inhibitor of JAK is or comprises Tofacitinib. In some embodiments, the small molecule modulator of S1P is or comprises Ozanimod.

### Overview

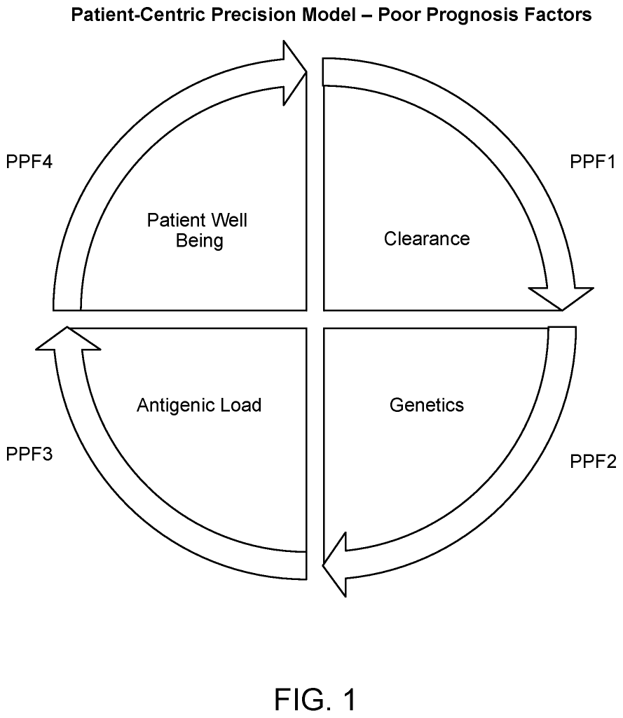

Disclosed herein, in some embodiments, are systems for determining a type or a dosage amount of a therapeutic agent disclosed herein, such as an anti-TNF therapy. FIG. 1 provides, accordingly to some embodiments disclosed herein, a model comprising one or more of four (4) “poor prognosis factors (PPF)” comprising: (1) a pharmacokinetic system factor (“PPF1”), (2) a genetic system factor (“PPF2”), (3) a antigenic load factor (“PPF3”), and a patient wellbeing factor (“PPF4”). In some embodiments, the model may comprise of the pharmacokinetic system factor, the genetic system factor, and the antigenic load factor. In some embodiments, the model may comprise of the pharmacokinetic system factor, the genetic system factor and the patient wellbeing factor.

In some embodiments, the pharmacokinetic system (PPF1) is configured to calculate an estimated clearance time of a biologic drug in the subject. In some embodiments, the biologic drug comprises an antibody or antigen-binding fragment thereof. In some embodiments, the biologic drug comprises an anti-Tumor Necrosis Factor (TNF) therapy. In some embodiments, the anti-TNF therapy comprises Remicade® (Infliximab), Enbrel® (Etanercept), Humira® (Adalimumab), Cimzia® (Certolizumab pegol), or Simponi® (Golimumab). In some embodiments, the anti-TNF therapy comprises Infliximab. In some embodiments, the anti-TNF therapy comprises an anti-TNF therapy other than Infliximab. In some embodiments, clearance of the biologic drug is estimated using the patient-centric precision model using albumin measured in a biological sample obtained from the subject and the weight of the subject as covariates in calculating the estimated clearance time of the therapeutic agent. A longer clearance time, relative to a healthy control, is indicative that the subject is in need of a higher dose of the therapeutic agent, or in some embodiments, a different therapeutic agent to effectively treat the immune-mediated inflammatory disease. Without being bound by any particular theory, immune-mediated inflammatory disease patients with higher clearance may exhibit higher clearance because they produce higher than normal neutralizing antibodies against the biologic drug (thereby clearing the biologic drug at a faster rate than normal). In some embodiments, the PPF1 scores are on a range of 0 to 2. In some embodiments, 0 indicates a faster time to disease remission, and 2 indicates a slower time to disease remission. In some embodiments, a PPF1 score of 0 is indicative of a “standard” response to the biologic drug (e.g., anti-TNF therapy). In some embodiments, a PPF1 score of 1 is indicative of a low likelihood of response to the biologic drug. In some embodiments, a PPF1 score of 2 is indicative of a very low likelihood of response to the biologic drug. In some embodiments, the patient-centric precision model predicts estimated clearance of the biologic by the subject using statistical techniques to analyze subject-specific data (e.g., weight or body mass index (BMI), level of albumin, and/or C-Reactive Protein (CRP) relative to a cutoff of about 3 mg/L) and model a simulation to estimate clearance of the biologic drug. In some embodiments, the statistical techniques comprise mixed effect modeling, such as nonlinear mixed-effect modeling (NONMEM).

In some embodiments, the genetic system (PPF2) is configured to analyze a genotype of the subject at a genetic locus associated with refractory disease, such as HLADQA1*05, to predict the time to remission following induction of a therapeutic agent to the subject for treatment of the immune-mediated inflammatory disease. In some embodiments, the genetic locus associated with refractory disease comprises HLADQA1*05. In some embodiments, the genetic locus associated with refractory disease comprises a genetic locus other than HLADQA1*05. In some embodiments, the genotype is analyzed using a statistical model disclosed herein. In some embodiments, the genetic system is configured to detect a genotype at single nucleotide variant rs2097432, located chromosome 6, nucleotide position 32622994 (GRCh38) or chromosome 6, nucleotide position 32590771 (GRCh37). In some embodiments, the nucleotide at nucleotide position 32622994 (GRCh38) or 32590771 (GRCh37) is a guanine (“G”). In some embodiments, the genotype comprises an SNV in linkage disequilibrium with rs2097432. Linkage disequilibrium may be evaluated based on a D′ value that is at least 0.20, or an r² value comprising at least 0.70, but preferably 0.85. In some embodiments, the genotype is homozygous for the risk allele, heterozygous for the risk allele, or homozygous for the non-risk allele. In some embodiments, the genotype for the risk allele may be homozygous (e.g., GG). In some embodiments, the genotype for the risk allele may be heterozygous (e.g., AG). In some embodiments, the genetic system receives genotype data for the subject from a genotyping device, such as a sequencer, a genotype array, or the like. In some embodiments, the “G” at nucleotide position 32622994 (GRCh38) or 32590771 (GRCh37) is associated an increased risk of developing antibodies to Infliximab or antibodies to adalimumab, based on the Personalising Anti-TNF Therapy in Crohn's Disease (PANTS) clinical study, which included a discovery cohort of 1240 biologic-naïve patients with Crohn's disease starting Infliximab or adalimumab and a replication cohort of 178 patients with IBD, which is described in Sazonovs A, Kennedy N A, Moutsianas L, et al; PANTS Consortium. HLA-DQA1*05 carriage associated with development of anti-drug antibodies to Infliximab and adalimumab in patients with Crohn's disease. Gastroenterology. 2020; 158(1):189-199, which is hereby incorporatedby reference in its entirety. The PANTS study included subjects of European descent, 40% of whom carry the risk allele (AG or GG). In the PANTS study, immunogenicity was defined as an antidrug antibody concentration of ≥10 AU/mL, irrespective of drug level, at 1 or more time points. The “G” at nucleotide position 32622994 (GRCh38) or 32590771 (GRCh37) is also associated with increased risk of loss of response to Infliximab, based on a separate retrospective study of 262 IBD patients on Infliximab therapy of which 40% identified as variant carriers (AG or GG), as described in Wilson A., Peel C., Wang Q., Pananos A D, Kim R B. HLADQA1*05 genotype predicts anti-drug antibody formation and loss of response during Infliximab therapy for inflammatory bowel disease. Aliment Pharmacol Ther. 2020:51(3); 356-363, which is hereby incorporated by reference in its entirety. In some embodiments, the PPF2 score of 0 is indicative that the subject has a homozygous non-risk genotype (e.g., HLA-DQA1*05 (AA)). In some embodiments, the PPF2 score of 1 is indicative that the subject has a heterozygous non-risk genotype/risk genotype (e.g., the subject is a “carrier” as used herein, e.g., HLA-DQA1*05 (AG)). In some embodiments, the PPF2 score of 2 is indicative that the subject has a homozygous risk genotype (e.g., HLA-DQA1*05 (GG)). In some embodiments, the scores herein may be assigned the opposite outcome, such as for example, assigning a PPF2 score of 2 to a homozygous non-risk genotype, and a PPF2 score of 0 to a homozygous risk genotype. In some embodiments, the genetic system (PPF2) utilizes a statistical algorithm. In some embodiments, the statistical algorithm is a logistic regression algorithm. In some embodiments, the logistic regression algorithm is a univariate logistic regression model. In some embodiments, PPF2 scores are on a range of 0 to 2, with 0 being the faster time to disease remission, and 2 being the slower time to disease remission.

In some embodiments, the antigenic load system is configured to determine a likelihood of response or non-response to a biologic drug by measuring an inflammatory load using levels of biomarkers such as c-reactive protein (CRP), and/or perinuclear antineutrophil cytoplasmic antibodies (pANCA), proteinase 3 (PR3), or antibodies against certain integrins. In some embodiments, a level of CRP is detected, and compared to a cutoff of 3 mg/L, with CRP>3 mg/L being a high antigenic load and CRP<3 mg/L being a normal antigenic load. In some embodiments, a presence or an absence of pANCA is detected. In some embodiments, a level of PR3 is detected. In some embodiments, a level of antibodies against certain integrins associated with inflammatory bowel disease (IBD) are detected, such as antibodies against: α4β1 (encoded by very late antigen-4, VLA-4), (integrin aE (encoded by integrin alpha-E, ITGAE) α4 (encoded by integrin alpha-4, ITGA4), β7 (encoded by integrin subunit Beta 7, ITGB7), β8 (encoded by integrin subunit Beta 8, ITGB8), integrin subunit alpha L (ITGAL), intercellular adhesion molecule 1 (ICAM1), any dimers thereof, or any combinations thereof. In some embodiments, the antigenic load system (PPF3) utilizes a statistical algorithm that applies weights to the multiple variables. In some embodiments, the statistical algorithm is a logistic regression algorithm. In some embodiments, the logistic regression algorithm is a multivariate logistic regression algorithm. In some embodiments, the weights applied are based on a factor associated with the expression level of the multiple variable. In some embodiments, a PPF3 score of 0 is indicative of a standard response to the anti-TNF therapy. In some embodiments, a PPF3 score of 1 is indicative e of a low response to the anti-TNF therapy. In some embodiments, a PPF3 score of 2 is indicative of very low response to the anti-TNF therapy. In some embodiments, a PPF3 score of 0 is indicative of an absence of Perinuclear anti-neutrophil cytoplasmic antibodies (pANCA), a level of proteinase 3 (PR3) in the 99^{th} percentile, and/or lower than normal levels of antibodies (e.g., autoantibodies against certain integrins associated with disease). In some embodiments, a PPF3 score of 2 is indicative of an presence of Perinuclear anti-neutrophil cytoplasmic antibodies (pANCA), a level of proteinase 3 (PR3) above the 99^{th} percentile, and/or higher than normal levels of antibodies (e.g., autoantibodies against certain integrins associated with disease).

In some embodiments, the scores herein may be assigned the opposite outcome, such as for example, assigning a PPF3 score of 2 to a standard response to the anti-TNF therapy, and a PPF3 score of 0 to a very low response to the anti-TNF therapy.

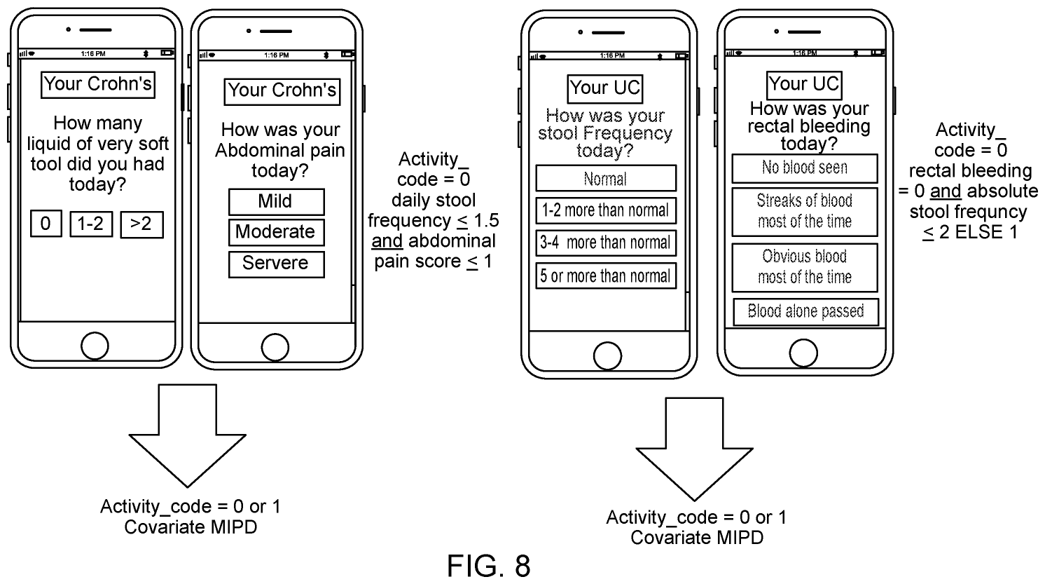

In some embodiments, the patient wellbeing system (PPF4) is configured to receive, from the subject, information related to the subject's symptoms and assign a score based on the information received. In some embodiments, the PPF4 score of 0 is indicative that the subject is not experiencing any symptom of the immune-mediated inflammatory disease. In some embodiments, the PPF4 score of 1 is indicative that the subject is experiencing at least one symptom of the immune-mediated inflammatory disease. In some embodiments, the scores herein may be assigned the opposite outcome, such as for example, a PPF4 score of 1 is indicative that the subject is not experiencing any symptom of the immune-mediated inflammatory disease, and a PPF4 score of 0 is indicative that the subject is experience at least one symptom of the immune-mediated inflammatory disease. In some embodiments, the symptoms include diarrhea, constipation, rectal bleeding, bloating, cramping, abdominal pain, fatigue, brain fog, and the like. As shown in FIG. 8, such information may be entered by the subject to their personal electronic device via a web or mobile-based Application displayed on a Graphical User Interface (GUI). The information, in some cases, are recorded daily by the subject. In other cases, the information is recorded monthly, annually, and so forth. In some embodiments, the information is transmitted to a server in a computer system disclosed herein, whether remote or otherwise, and then transmitted to one or more computer processors in the patient wellbeing system for analysis.

In some embodiments, a combined PPF score may comprise two or more of the PPF1, PPF2, and PPF3, PPF4 scores. In some embodiments, the combined PPF score comprises the PPF1 and PPF2 scores. In some embodiments, the PPF score comprises the PPF1 and PPF3 scores. In some embodiments, the PPF score comprises the PPF1 and PPF4 scores. In some embodiments, the combined PPF score comprises the PPF2 and PPF3 scores. In some embodiments, the combined PPF score comprises the PPF2 and PPF4 scores. In some embodiments, the combined PPF score comprises the PPF3 and PPF4 scores. In some embodiments, the combined PPF score comprises three or more of the PPF1, PPF2, and PPF3, PPF4 scores. In some embodiments, the combined PPF score comprises PPF1, PPF2, and PPF3, PPF4 scores.

With reference to the scores disclosed herein, it is contemplated that the scores may be flipped such that a PPF score of 0 may be assigned an outcome characterized by a faster time to remission, and a PPF score of 1 or 2 may be assigned an outcome characterized by a slower time to remission.

### Subject

In some embodiments, the subject may be a subject diagnosed with the immune-mediated inflammatory disease, such an inflammatory bowel disease. In some embodiments, the inflammatory bowel disease comprises Crohn's disease (CD) or ulcerative colitis (UC). In some embodiments, the subject comprises a mammal, such as a human or a farm animal. In some embodiments, the subject comprises an adult, such as for example, ages 18 and older. In some embodiments, the subject comprises an adolescent or a child, such as for example ages 1-17.

The subject disclosed herein can be a mammal, such as for example a mouse, rat, guinea pig, rabbit, non-human primate, or farm animal. In some instances, the subject is human. In some instances, the subject is suffering from a symptom related to a disease or condition disclosed herein (e.g., abdominal pain, cramping, diarrhea, rectal bleeding, fever, weight loss, fatigue, loss of appetite, dehydration, and malnutrition, anemia, or ulcers).

In some embodiments, the subject is susceptible to, or is inflicted with, thiopurine toxicity, or a disease caused by thiopurine toxicity (such as pancreatitis or leukopenia). The subject may experience, or is suspected of experiencing, non-response or loss-of-response to a treatment. In some embodiments, the may be treatment with a biologic drug disclosed herein (e.g., anti-TNF alpha therapy, anti-α4β7 therapy (e.g., vedolizumab), anti-IL12p40 therapy (e.g., ustekinumab)), corticosteroid (e.g., budesonide), chemotherapy,).

In some embodiments, the subject has one or more diseases. The disease may be a disease disclosed herein. In some embodiments, the subject has a recursive disease. In some embodiments, the subject has a disease in clinical remission. In some embodiments, the subject has a disease that is not in clinical remission. In some embodiments, the subject is responsive to a first line therapy, such as, for example, an anti-TNF inhibitor. In some embodiments, the subject is not responsive to a first line therapy, such as, for example, an anti-TNF inhibitor. In some embodiments from the disease comprises an immune-mediated inflammatory disease, such as, but not limited to, inflammatory bowel disease (IBD), rheumatoid arthritis (RA), cytokine release syndrome, multiple sclerosis (MS), ankylosing spondylitis (AS), lupus, plaque psoriasis, atopic dermatitis, gout, or migraine. In some cases, the IBD comprises Crohn's disease (CD). In some cases, the IBD comprises ulcerative colitis (UC).

In some embodiments, the subject has a disease that has a disease severity categorized by an index or score. In some embodiments, the disease severity is classified according to a severity of illness (SOI). In some embodiments, the disease severity is classified according to a clinical disease activity index (CDAI) or Crohn's disease activity index (CDAI). In some embodiments, the disease severity is classified by a simple disease activity index (SDAI). In some embodiments, the subject has a disease severity that is classified as remission, mildly active, moderately active, severely active, fulminant disease, or any combination thereof. In some embodiments, the severity of the disease comprises a disease remission, a disease recurrence, a disease type, or any combination thereof. In some embodiments, the severity of a symptom of the disease comprises a frequency of the symptom, a type of the symptom, or a combination thereof.

In some embodiments, the subject has one or more symptoms. A symptom may comprise, but is not limited to, fever, cold, chills, sore throat, cough, fatigue, rashes, headache, congestion, nausea, vomiting, rectal bleeding, weight loss, appetite, constipation, sweating, sneezing, wheezing, shortness of breath, high blood pressure, pain (e.g., abdominal pain, join pain, food pain, etc.), swelling (e.g., foot swelling, leg swelling, etc.), dizziness, or any combination thereof. In some embodiments, the symptom is a secondary immune-mediated condition, such as, for example, eczema.

In some embodiments, the subject is a baby, child, adolescence, adult, or senior. In some embodiments, the subject is in their teens, 20s, 30s, 40s, 50s, 60s, 70s, 80s, or 90s. In some embodiments, the subject is a female or a male. In some embodiments, the subject has a smoking history. In some embodiments, the subject has an alcohol history.

### Medical Professional

The results of the patient-centric precision dosing tool disclosed herein, in some embodiments, may be communicated to a medical professional. In some embodiments, the medical professional is a doctor, nurse, physician assistant, or the like. In some embodiments, the medical professional is a gastroenterologist, dermatologist, rheumatologist, or neurologist, or a combination thereof. In some embodiments, the medical professional is a pharmacist.

### Systems

The systems disclosed herein, in some embodiments, are or comprise one or more computer systems that are programmed or otherwise configured to implement methods provided herein, such as, for example, methods for calculating a combined PPF score for a subject. In some embodiments, the combined PPF score is predictive of a therapeutic response to a therapeutic agent disclosed herein, such as for example, an anti-TNF therapy. In some embodiments, the combined PPF score is predictive of a particular treatment course (e.g., dose, inter-dose interview) to achieve and/or sustain disease remission in the subject.

Aspects disclosed herein comprise, in some embodiments, computer-implemented systems comprising a computing device comprising at least one processor, an operating system configured to perform executable instructions, a memory, and a computer program including instructions executable by the computing device to create an application comprising: (a) a first software module configured to calculate a poor prognosis factor 1 (PPF1) score for the subject comprising: (i) receiving a level of albumin from a biological sample obtained from a subject, and a weight of the subject; (ii) calculating a rate of which a biologic drug is estimated to be cleared from the subject using a first statistical model based, at least in part, on the level of the albumin measured in the biological sample, and the weight of the subject; and (iii) comparing the rate to a predetermined threshold for the anti-TNF therapy, to produce an estimated clearance score on a scale of 0 to 1, wherein 0 is indicative of a low rate of clearance below the predetermined threshold, and 1 is indicative of a high rate of clearance above the predetermined threshold; (b) a second software module configured to calculate a poor prognosis factor 2 (PPF 2) score for the subject comprising: (i) receiving a genotype of the subject at single nucleotide variant (SNV) comprising rs2097432 or a SNV in linkage disequilibrium therewith; and (ii) calculating a PPF2 score using a second statistical model, wherein the PPF2 score is on a scale of 0 to 2, wherein 0 is indicative of the genotype that is homozygous non-risk, 1 is indicative of the genotype that is heterozygous risk at rs2097432, and 2 comprises a presence of two genetic loci of said genotype; and (c) a third software module configured to calculate a poor prognosis factor 3 (PPF3) score or the subject, comprising: (i) calculating a PPF3 score using a third statistical model based on a presence or a level of one or more serological marker (e.g., pANCA, PR3) detected in the biological sample obtained from the subject, wherein the PPF3 score is on a scale of 0 to 2, wherein 0 is indicative of a high likelihood of a standard response to the biologic drug, 1 is indicative of a low likelihood of response to the biologic drug, and 2 is indicative of a very low likelihood of response to the biologic drug; and (c) a forth software module configured to calculate a poor prognosis factor 4 (PPF 4) for the subject comprising: (i) receiving wellbeing data from said subject comprising one or more symptoms related to the immune-mediated inflammatory disease; (ii) calculating a wellbeing score with a fourth statistical model of 0 or 1 based on the wellbeing data received, wherein 0 is indicative of an improvement of at least one symptom of the one or more symptoms, and 1 is indicative of no improvement in the at least one symptom of the one or more symptoms; and (d) a fifth software module configured to calculate a combined PPF score comprising at least two of the PPF1 score, the PPF2 score, the PPF3 score, and the PPF4 score to predict a time to remission following administration of the anti-TNF therapy to the subject. In some embodiments, the weight of the subject and the level of albumin values are log transformed and divided by the typical value from a reference population. In some embodiments, the typical value for weight and level of albumin is an average (mean) value or a median value. In some embodiments, the reference population contains reference individuals with the same disease or condition as the subject that have received an anti-TNF therapy to treat the disease or the condition.

Aspects disclosed herein provide, in some embodiments, computer-implemented methods for calculating a combined poor prognosis factor (PPF) score for a subject, the method comprising: (a) calculating, by a computer, a poor prognosis factor 1 (PPF1) score for the subject comprising: (i) receiving a level of albumin from a biological sample obtained from a subject, and a weight of the subject; (ii) calculating a rate of which a biologic drug is estimated to be cleared from the subject using a first statistical model based, at least in part, on the level of the albumin measured in the biological sample and the weight of the subject; and (iii) comparing the rate to a predetermined threshold for the biologic drug, to produce an estimated clearance score on a scale of 0 to 1, wherein 0 is indicative of a low rate of clearance below the predetermined threshold, and 1 is indicative of a high rate of clearance above the predetermined threshold; (b) calculating, by a computer, a poor prognosis factor 2 (PPF 2) score for the subject comprising: (i) receiving a genotype of the subject at single nucleotide variant (SNV) comprising rs2097432 or a SNV in linkage disequilibrium therewith; and (ii) calculating a PPF2 score using a second statistical model, wherein the PPF2 score is on a scale of 0 to 2, wherein 0 is indicative of the genotype that is homozygous non-risk, 1 is indicative of the genotype that is heterozygous risk at rs2097432, and 2 comprises a presence of two genetic loci of said genotype; and (c) calculating, by a computer, a poor prognosis factor 3 (PPF3) score or the subject, comprising: (i) calculating a PPF3 score using a third statistical model based on a presence or a level of one or more serological marker (e.g., pANCA, PR3) detected in the biological sample obtained from the subject, wherein the PPF3 score is on a scale of 0 to 2, wherein 0 is indicative of a high likelihood of a standard response to the biologic drug, 1 is indicative of a low likelihood of response to the biologic drug, and 2 is indicative of a very low likelihood of response to the biologic drug; and (e) calculating, by a computer, a poor prognosis factor 4 (PPF 4) for the subject comprising: (i) receiving wellbeing data from said subject comprising one or more symptoms related to the immune-mediated inflammatory disease; (ii) calculating a wellbeing score with a fourth statistical model of 0 or 1 based on the wellbeing data received, wherein 0 is indicative of an improvement of at least one symptom of the one or more symptoms, and 1 is indicative of no improvement in the at least one symptom of the one or more symptoms; and (f) calculating, by a computer, a combined PPF score comprising at least two of the PPF1 score, the PPF2 score, the PPF3 score, and the PPF4 score to predict a time to remission following administration of the biologic drug to the subject. In some embodiments, the weight of the subject and the level of albumin values are log transformed and divided b y the typical value from a reference population. In some embodiments, the typical value for weight and level of albumin is an average (mean) value or a median value. In some embodiments, the reference population contains reference individuals with the same disease or condition as the subject that have received an biologic drug to treat the disease or the condition.

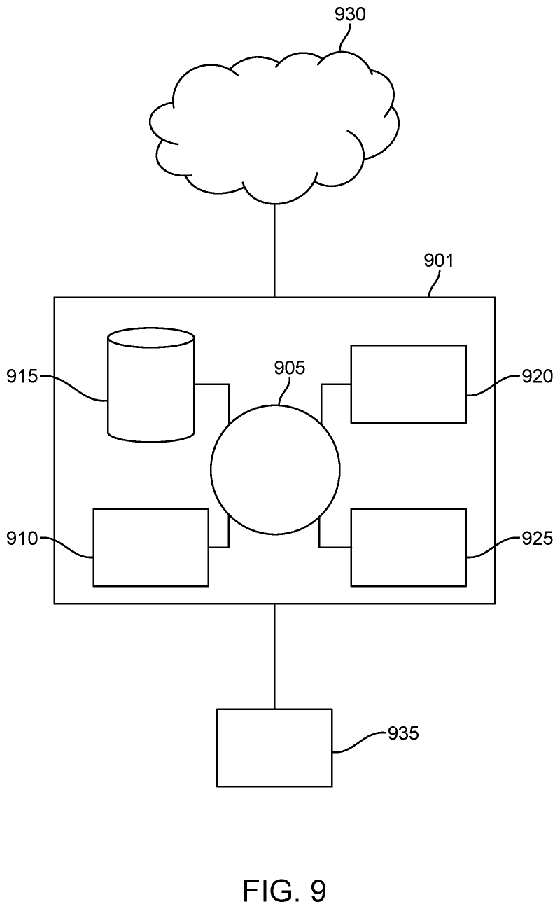

An example of such a computer system is shown in FIG. 9. As shown in FIG. 9, the computer system 901 includes a central processing unit (CPU, also “processor” and “computer processor” herein) 905, which can be a single core or multi core processor, or a plurality of processors for parallel processing. The computer system 901 also includes memory or memory location 910 (e.g., random-access memory, read-only memory, flash memory), electronic storage unit 915 (e.g., hard disk), communication interface 920 (e.g., network adapter) for communicating with one or more other systems, and peripheral devices 925, such as cache, other memory, data storage or electronic display adapters. The memory 910, storage unit 915, interface 920 and peripheral devices 925 are in communication with the CPU 905 through a communication bus (solid lines), such as a motherboard. The storage unit 915 can be a data storage unit (or data repository) for storing data. The computer system 901 can be operatively coupled to a computer network (“network”) 930 with the aid of the communication interface 920. The network 930 can be the Internet, an internet or extranet, or an intranet or extranet that is in communication with the Internet. The network 930 in some cases is a telecommunication or data network. The network 930 can include one or more computer servers, which can enable distributed computing, such as cloud computing. The network 930, in some cases with the aid of the computer system 901, can implement a peer-to-peer network, which may enable devices coupled to the computer system 901 to behave as a client or a server.

The CPU 905 can execute a sequence of machine-readable instructions, which can be embodied in a program or software. In some embodiments, the machine-readable instructions comprise a machine learning algorithm. In some embodiments, the machine learning algorithm comprises one or more of a linear regression, a logistic regression, a decision tree, a support vector machine (SVM), naive Bayes, k-nearest neighbors (KNN), K-Means clustering, a random Forest, or a neural network. The instructions may be stored in a memory location, such as the memory 910 Examples of operations performed by the CPU 905 can include fetch, decode, execute, and writeback.

The storage unit 915 can store files, such as drivers, libraries and saved programs. The storage unit 915 can store user data, e.g., user preferences and user programs. The computer system 901 in some cases can include one or more additional data storage units that are external to the computer system 901, such as located on a remote server that is in communication with the computer system 901 through an intranet or the Internet.

The computer system 901 can communicate with one or more remote computer systems through the network 930. For instance, the computer system 901 can communicate with a remote computer system of a user (e.g., operator). Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system 901 via the network 930.

Methods as described herein can be implemented byway of machine (e.g., computer processor) executable code stored on an electronic storage location of the computer system 901, such as, for example, on the memory 910 or electronic storage unit 915. The machine executable or machine readable code can be provided in the form of software. During use, the code can be executed by the processor 905. In some cases, the code can be retrieved from the storage unit 915 and stored on the memory 99 for ready access by the processor 905. In some situations, the electronic storage unit 915 can be precluded, and machine-executable instructions are stored on memory 99.

The code can be pre-compiled and configured for use with a machine have a processer adapted to execute the code or can be compiled during runtime. The code can be supplied in a programming language that can be selected to enable the code to execute in a pre-compiled or as-compiled fashion.

Aspects of the systems provided herein, such as the computer system 901, can be embodied in programming. Various aspects of the technology may be thought of as “products” or “articles of manufacture” in the form of machine (or processor) executable code or associated data that is carried on or embodied in a type of machine readable medium. In some embodiments, the executable code or associated data comprise a machine learning algorithm. In some embodiments, the machine learning algorithm comprises one or more of a linear regression, a logistic regression, a decision tree, a support vector machine (SVM), naive Bayes, k-nearest neighbors (KNN), K-Means clustering, a random Forest, or a neural network. Machine-executable code can be stored on an electronic storage unit, such memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Disclosed herein, in some embodiments are computer-implemented methods of training a machine learning algorithm that determines a poor prognosis factor (PPF) profile of a subject with an inflammatory bowel disease, the method comprising: (a) receiving genotype data, clearance data, serological marker data and/or wellbeing data from a database comprising data collected from patients with one or more inflammatory bowel diseases; (b) creating a first training set comprising patient covariate data received in the genotype data, the clearance data, the serological marker data, and/or the wellbeing data; (c) training the machine learning algorithm using the first training set; (d) creating a second training set comprising newly received patient covariate data; (e) training the machine learning algorithm using the second training set; and (f) applying the machine learning algorithm to determine the PPF profile of the subject with the inflammatory bowel disease, wherein the PPF profile comprises of a time to remission of the subject following administration of a biologic drug to the subject. In some embodiments, the biologic drug comprises an anti-TNF therapy.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage m edium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer m ay read programming code or data. Many of these forms of computer readable media may be involved in carrying one or more sequences of one or more instructions to a processor for execution. The computer system 901 can include or be in communication with an electronic display 935 that comprises a user interface (UI) for providing, for example, an output or readout of a nucleic acid sequencing instrument coupled to the computer system 901. Such readout can include a nucleic acid sequencing readout, such as a sequence of nucleic acid bases that comprise a given nucleic acid sample. The UI may also be used to display the results of an analysis making use of such readout. Examples of UI's include, without limitation, a graphical user interface (GUI) and web-based user interface. The electronic display 935 can be a computer monitor, or a capacitive or resistive touchscreen.

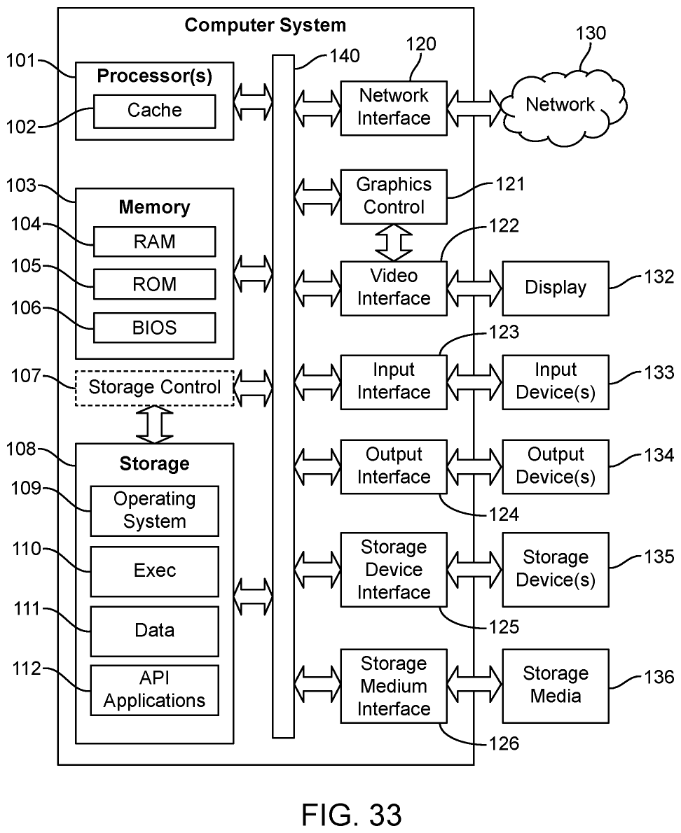

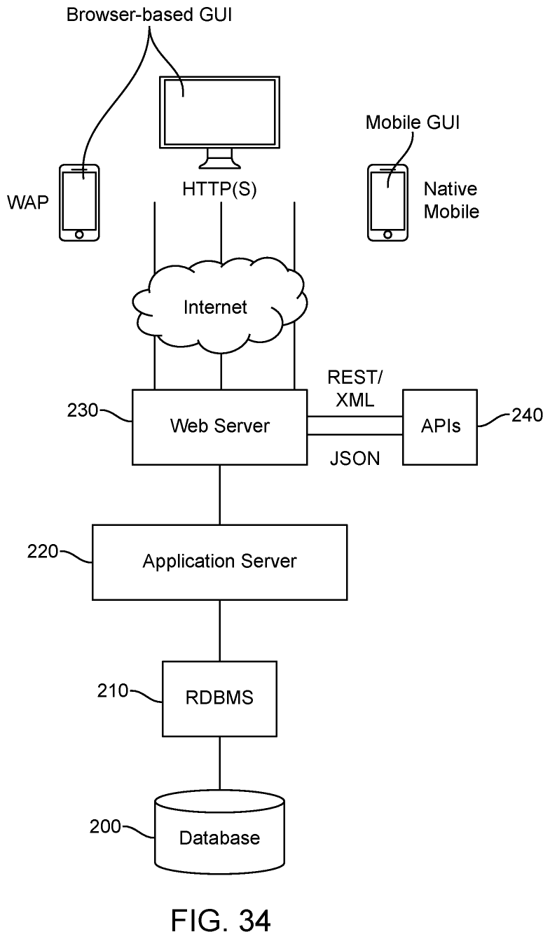

Referring to FIG. 33, a block diagram is shown depicting an exemplary machine that includes a computer system 100 (e.g., a processing or computing system) within which a set of instructions can execute for causing a device to perform or execute any one or more of the aspects and/or methodologies for static code scheduling of the present disclosure. The components in FIG. 34 are examples only and do not limit the scope of use or functionality of any hardware, software, embedded logic component, or a combination of two or more such components implementing particular embodiments.

Computer system 100 may include one or more processors 101, a memory 103, and a storage 108 that communicate with each other, and with other components, via a bus 140. The bus 140 may also link a display 132, one or more input devices 133 (which may, for example, include a personal electronic device, a health tracking device, a keypad, a keyboard, a mouse, a stylus, etc.), one or more output devices 134, one or more storage devices 135, and various tangible storage media 136. All of these elements may interface directly or via one or more interfaces or adaptors to the bus 140. For instance, the various tangible storage media 136 can interface with the bus 140 via storage medium interface 126. Computer system 100 may have any suitable physical form, including but not limited to one or more integrated circuits (ICs), printed circuit boards (PCBs), mobile handheld devices (such as mobile telephones or PDAs), laptop or notebook computers, distributed computer systems, computing grids, or servers.

Computer system 100 includes one or more processor(s) 101 (e.g., central processing units (CPUs), general purpose graphics processing units (GPGPUs), or quantum processing units (QPUs)) that carry out functions. Processor(s) 101 optionally contains a cache memory unit 102 for temporary local storage of instructions, data, or computer addresses. Processor(s) 101 are configured to assist in execution of computer readable instructions. Computer system 100 may provide functionality for the components depicted in FIG. 34 as a result of the processor(s)101 executing non-transitory, processor-executable instructions embodied in one or more tangible computer-readable storage media, such as memory 103, storage 108, storage devices 135, and/or storage medium 136. The computer-readable media may store software that implements particular embodiments, and processor(s) 101 may execute the software. Memory 103 may read the software from one or more other computer-readable media (such as mass storage device(s) 135, 136) or from one or more other sources through a suitable interface, such as network interface 120. The software may cause processor(s) 101 to carry out one or more processes or one or more steps of one or more processes described or illustrated herein. Carrying out such processes or steps may include defining data structures stored in memory 103 and modifying the data structures as directed by the software.

The memory 103 may include various components (e.g., machine readable media) including, but not limited to, a random access memory component (e.g., RAM 104) (e.g., static RAM (SRAM), dynamic RAM (DRAM), ferroelectric random access memory (FRAM), phase-change random access memory (PRAM), etc.), a read-only memory component (e.g., ROM 105), and any combinations thereof. ROM 105 may act to communicate data and instructions unidirectionally to processor(s) 101, and RAM 104 may act to communicate data and instructions bidirectionally with processor(s) 101. ROM 105 and RAM 104 may include any suitable tangible computer-readable media described below. In one example, a basic input/output system 106 (BIOS), including basic routines that help to transfer information between elements within computer system 100, such as during start-up, may be stored in the memory 103.

Fixed storage 108 is connected bidirectionally to processor(s) 101, optionally through storage control unit 107. Fixed storage 108 provides additional data storage capacity and may also include any suitable tangible computer-readable media described herein. Storage 108 may be used to store operating system 109, executable(s) 110, data 111, applications 112 (application programs), and the like. In some embodiments, the data 111 comprises electronic medical record (EMR) data. Storage 108 can also include an optical disk drive, a solid-state memory device (e.g., flash-based systems), or a combination of any of the above. Information in storage 108 may, in appropriate cases, be incorporated as virtual memory in memory 103.

In one example, storage device(s) 135 may be removably interfaced with computer system 100 (e.g., via an external port connector (not shown)) via a storage device interface 125. Particularly, storage device(s) 135 and an associated machine-readable medium may provide non-volatile and/or volatile storage of machine-readable instructions, data structures, program modules, and/or other data for the computer system 100. In one example, software may reside, completely or partially, within a machine-readable medium on storage device(s) 135. In another example, software may reside, completely or partially, within processor(s) 101.

Bus 140 connects a wide variety of subsystems. Herein, reference to a bus may encompass one or more digital signal lines serving a common function, where appropriate. Bus 140 may be any of several types of bus structures including, but not limited to, a memory bus, a memory controller, a peripheral bus, a local bus, and any combinations thereof, using any of a variety of bus architectures. As an example and not by way of limitation, such architectures include an Industry Standard Architecture (ISA) bus, an Enhanced ISA (EISA) bus, a Micro Channel Architecture (MCA) bus, a Video Electronics Standards Association local bus (VLB), a Peripheral Component Interconnect (PCI) bus, a PCI-Express (PCI-X) bus, an Accelerated Graphics Port (AGP) bus, HyperTransport (HTX) bus, serial advanced technology attachment (SATA) bus, and any combinations thereof.

Computer system 100 may also include an input device 133. In one example, a user of computer system 100 may enter commands and/or other information into computer system 100 via input device(s) 133. Examples of an input device(s) 133 include, but are not limited to, an alpha-numeric input device (e.g., a keyboard), a pointing device (e.g., a mouse or touchpad), a touchpad, a touch screen, a multi-touch screen, a joystick, a stylus, a gamepad, an audio input device (e.g., a microphone, a voice response system, etc.), an optical scanner, a video or still image capture device (e.g., a camera), and any combinations thereof. Further examples of input devices are provided herein. In some embodiments, the input device is a Kinect, Leap Motion, or the like. Input device(s) 133 may be interfaced to bus 140 via any of a variety of input interfaces 123 (e.g., input interface 123) including, but not limited to, serial, parallel, game port, USB, FIREWIRE, THUNDERBOLT, or any combination of the above.

In particular embodiments, when computer system 100 is connected to network 130, computer system 100 may communicate with other devices, specifically mobile devices and enterprise systems, distributed computing systems, cloud storage systems, cloud computing systems, and the like, connected to network 130. Communications to and from computer system 100 may be sent through network interface 120. For example, network interface 120 may receive incoming communications (such as requests or responses from other devices) in the form of one or more packets (such as Internet Protocol (IP) packets) from network 130, and computer system 100 may store the incoming communications in memory 103 for processing. Computer system 100 may similarly store outgoing communications (such as requests or responses to other devices) in the form of one or more packets in memory 103 and communicated to network 130 from network interface 120. Processor(s) 101 may access these communication packets stored in memory 103 for processing.

Examples of the network interface 120 include, but are not limited to, a network interface card, a modem, and any combination thereof. Examples of a network 130 or network segment 130 include, but are not limited to, a distributed computing system, a cloud computing system, a wide area network (WAN) (e.g., the Internet, an enterprise network), a local area network (LAN) (e.g., a network associated with an office, a building, a campus or other relatively small geographic space), a telephone network, a direct connection between two computing devices, a peer-to-peer network, and any combinations thereof. A network, such as network 130, may employ a wired and/or a wireless mode of communication. In general, any network topology may be used.

Information and data can be displayed through a display 132. Examples of a display 132 include, but are not limited to, a cathode ray tube (CRT), a liquid crystal display (LCD), a thin film transistor liquid crystal display (TFT-LCD), an organic liquid crystal display (OLED) such as a passive-matrix OLED (PMOLED) or active-matrix OLED (AMOLED) display, a plasma display, and any combinations thereof. The display 132 can interface to the processor(s) 101, memory 103, and fixed storage 108, as well as other devices, such as input device(s) 133, via the bus 140. The display 132 is linked to the bus 140 via a video interface 122, and transport of data between the display 132 and the bus 140 can be controlled via the graphics control 121. In some embodiments, the display is a video projector. In some embodiments, the display is a head-mounted display (HMD) such as a VR headset. In further embodiments, suitable VR headsets include, by way of non-limiting examples, HTC Vive, Oculus Rift, Samsung Gear VR, MicrosoftHoloLens, Razer OSVR, FOVE VR, Zeiss VR One, Avegant Glyph, Freefly VR headset, and the like. In still further embodiments, the display is a combination of devices such as those disclosed herein.

In some embodiments, the display 132 may display electronic medical record (EMR) data, user data, a treatment recommendation, facilitate communications with a user, or any combination thereof. In some embodiments, the display may facilitate communication with a user by providing a display by which a user can input information. The EMR data, user data, or input information may comprise any data related to a user's health, diet, exercise, or any combination thereof. Non-limiting examples of data are further provided herein. In some embodiments the display 132 may be communicated to user. In some embodiments, the user may comprise a patient or a subject. The patient or a subject may use the display to track their health, diet, exercise, wellbeing, or any combination thereof. In some embodiments, the user may comprise a healthcare professional, a clinician, a physician, a nurse, a pharmacist, a healthcare administrator, a technician, a veterinarian, a healthcare assistant, a therapist, a radiographer, a dentist, a surgeon, an optometrist, or any variation thereof. The healthcare professional may use the display to review, track, and/or monitor a patient's health, diet, exercise, wellbeing, or any combination thereof. The healthcare professional, for example, a physician, may further use information on the display 132 to evaluate treatments for a patient or a subject. The treatments for a patient may be evaluated based on displayed treatment recommendations and/or patient data. The healthcare professional, for example, a pharmacist, may further use information on the display 132 to fill a prescription for a patient or a subject. In some embodiments, the display 132 may display a test report.

In addition to a display 132, computer system 100 may include one or more other peripheral output devices 134 including, but not limited to, an audio speaker, a printer, a storage device, and any combinations thereof. Such peripheral output devices may be connected to the bus 140 via an output interface 124. Examples of an output interface 124 include, but are not limited to, a serial port, a parallel connection, a USB port, a FIREWIRE port, a THUNDERBOLT port, and any combinations thereof.

In addition or as an alternative, computer system 100 may provide functionality as a result of logic hardwired or otherwise embodied in a circuit, which may operate in place of or together with software to execute one or more processes or one or more steps of one or more processes described or illustrated herein. Reference to software in this disclosure may encompass logic, and reference to logic may encompass software. Moreover, reference to a computer-readable medium may encompass a circuit (such as an IC) storing software for execution, a circuit embodying logic for execution, or both, where appropriate. The present disclosure encompasses any suitable combination of hardware, software, or both.

Those of skill in the art will appreciate that the various illustrative logical blocks, modules, circuits, and algorithm steps described in connection with the embodiments disclosed herein may be implemented as electronic hardware, computer software, or combinations of both. To clearly illustrate this interchangeability of hardware and software, various illustrative components, blocks, modules, circuits, and steps have been described above generally in terms of their functionality.

The various illustrative logical blocks, modules, and circuits described in connection with the embodiments disclosed herein may be implemented or performed with a general purpose processor, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A general purpose processor may be a microprocessor, but in the alternative, the processor may be any conventional processor, controller, microcontroller, or state machine. A processor may also be implemented as a combination of computing devices, e.g., a combination of a DSP and a microprocessor, a plurality of microprocessors, one or more microprocessors in conjunction with a DSP core, or any other such configuration.

The steps of a model, method and/or algorithm described in connection with the embodiments disclosed herein may be embodied directly in hardware, in a software module executed by one or more processor(s), or in a combination of the two. A software module may reside in RAM memory, flash memory, ROM memory, EPROM memory, EEPROM memory, registers, hard disk, a removable disk, a CD-ROM, or any other form of storage medium known in the art. An exemplary storage medium is coupled to the processor such the processor can read information from, and write information to, the storage medium. In the alternative, the storage medium may be integral to the processor. The processor and the storage medium may reside in an ASIC. The ASIC may reside in a user terminal. In the alternative, the processor and the storage medium may reside as discrete components in a user terminal.

In accordance with the description herein, suitable computing devices include, by way of non-limiting examples, server computers, desktop computers, laptop computers, notebook computers, sub-notebook computers, netbook computers, netpad computers, set-top computers, media streaming devices, handheld computers, Internet appliances, mobile smartphones, tablet computers, personal digital assistants, video game consoles, and vehicles. Those of skill in the art will also recognize that select televisions, video players, and digital music players with optional computer network connectivity are suitable for use in the system described herein. Suitable tablet computers, in various embodiments, include those with booklet, slate, and convertible configurations, known to those of skill in the art.

In some embodiments, the computing device includes an operating system configured to perform executable instructions. The operating system is, for example, software, including programs and data, which manages the device's hardware and provides services for execution of applications. Those of skill in the art will recognize that suitable server operating systems include, by way of non-limiting examples, FreeBSD, OpenBSD, NetBSD®, Linux, Apple® Mac OS X Server®, Oracle® Solaris®, Windows Server®, and Novell® NetWare®. Those of skill in the art will recognize that suitable personal computer operating systems include, by way of non-limiting examples, Microsoft® Windows®, Apple® Mac OS X®, UNIX®, and UNIX-like operating systems such as GNU/Linux®. In some embodiments, the operating system is provided by cloud computing. Those of skill in the art will also recognize that suitable mobile smartphone operating systems include, by way of non-limiting examples, Nokia® Symbian® OS, Apple® iOS®, Research In Motion® BlackBerry OS®, Google® Android®, Microsoft® Windows Phone® OS, Microsoft® Windows Mobile® OS, Linux®, and Palm® WebOS®. Those of skill in the art will also recognize that suitable media streaming device operating systems include, by way of non-limiting examples, Apple TV®, Roku®, Boxee®, Google TV®, Google Chromecast®, Amazon Fire®, and Samsung® HomeSync®. Those of skill in the art will also recognize that suitable video game console operating systemsinclude, by way of non-limiting examples, Sony® PS3®, Sony® PS4®, Microsoft® Xbox 360®, Microsoft Xbox One, Nintendo® Wii®, Nintendo® Wii U®, and Ouya®.

### Models

The models of the present disclosure may be implemented on a hardware, software, or combination thereof described herein. In some embodiments, a model may comprise one or more algorithms for treating a disease in a subject. The one or more algorithms may generally comprise a statistical method, a numerical method, or a machine learning method. The model may utilize one or more algorithms to analyze a biological sample obtained from a subject with a disease, such as those described herein. Analyzing a biological sample may comprise quantifying one or more analyte s or biomarkers in a biological sample, which may include, but is not limited to, a level of biologic drug, a level of autoantibodies against the biologic drug, a level of albumin, a level of C-reactive protein (CRP), a level of a serological marker (e.g., PR3, antibodies against certain integrins), a presence or an absence of pANCA, a presence or absence of one or more genotypes at the at a single nucleotide variant (SNV) comprising rs2097432 or a SNV in linkage disequilibrium therewith, or any combination thereof. In some embodiments, the biologic drug may comprise an antibody or an antigen-binding fragment. In some embodiments, the biologic drug comprises a monoclonal antibody, such as for example, adalimumab (ADA) or Infliximab (IFX). In some embodiments, the one or more analytes may comprise a biological molecule whose concentration is correlated to the concentration of the biologic drug or autoantibodies against the biologic drug.

In some embodiments, the model utilizes one or more algorithms for determining a likelihood of achieving disease remission in a subject. In some embodiments, the model comprises a Markov Chain Monte Carlo (MCMC) simulation. In some embodiments, the model comprises a non-linear mixed effects (NLME) model. In some embodiments, the model comprises a machine learning model. In some embodiments, the machine learning model comprises a deep learning model. In some embodiments, the model comprises Bayesian assimilation. In some embodiments, the one or more algorithms comprises a Naive Bayes classifier algorithm. In some embodiments, the one or more algorithms comprises a Metropolis Hastings algorithm. In some embodiments, the one or more algorithms comprises an supervised, semi-supervised, or unsupervised learning algorithm. In some embodiments, the one or more algorithms comprises a clustering or a classification algorithm. In some embodiments, the one or more algorithms comprises a neural network.

In some instances, the algorithm comprises a trained machine learning algorithm. In some instances, the algorithm comprises a statistical modeling algorithm. In some instances, the statistical modeling algorithm is a Markov chain Monte Carlo algorithm. In some instances, the statistical modeling algorithm is a Metropolis-Hastings algorithm. As used herein, Markov chain Monte Carlo (MCMC) methods comprise a class of algorithms for sampling from a probability distribution. By constructing a Markov chain that has the desired distribution as its equilibrium distribution, one can obtain a sample of the desired distribution by recording states from the chain. The more steps are included, the more closely the distribution of the sample matches the actual desired distribution. Various algorithms exist for constructing chains, including the Metropolis-Hastings algorithm. As used herein, the Metropolis-Hastings algorithm is a Markov chain Monte Carlo (MCMC) method for obtaining a sequence of random samples from a probability distribution from which direct sampling is difficult. This sequence can be used to approximate the distribution (e.g. to generate a histogram) or to compute an integral (e.g. an expected value). Metropolis-Hastings and other MCMC algorithms are generally used for sampling from multi-dimensional distributions, especially when the number of dimensions is high. For single-dimensional distributions, there are usually other methods (e.g. adaptive rejection sampling) that can directly return independent samples from the distribution, and these are free from the problem of autocorrelated samples that is inherent in MCMC methods.

In some cases, the at least one parameter measured from the subject comprises at least one of level of the biomarkers disclosed herein (e.g., genetic marker, serological marker, or other biomarker). In some cases, the method further comprises assaying or having assayed a biological sample obtained from the subject to measure the at least one parameter measured from the subject's biological sample (e.g., blood, saliva, urine, spinal fluid, tissue sample, etc.). In some cases, the at least one parameter measured from the subject's biological sample comprises albumin, C-reactive protein (CRP), pANCA, PR3, antibodies against: α4β1, αE, α4, β7, β8, ITGAL, ICAM1, any dimers thereof, or any combinations thereof, or a genotype at rs2097432 or a SNV in linkage disequilibrium therewith. In some cases, the subject is suffering from an immune-mediated inflammatory disease, such as, for example, an inflammatory bowel disease (e.g., CD or UC), multiple sclerosis, rheumatoid arthritis, ankylosing spondylitis, lupus, plaque psoriasis, atopic dermatitis, gout, migraine, or a combination thereof. In some cases, the clinical remission status is obtained from Electronic health record (“EMR”) data.

In some embodiments, an algorithm may determine a likelihood of achieving disease remission, at least in part on a level of one or more analytes or biomarkers (e.g., albumin, CRP, biologic drug, autoantibodies, genotypes, etc.) obtained from the subject. In some embodiments, an algorithm may determine a likelihood of achieving a disease remission based, at least in part on a current dose of the biologic drug and an current inter-dose interval of the biologic drug. The algorithm may determine the likelihood by determining whether the subject has a poor prognostic factor (PPF). In some embodiments, the PPF is a pharmacokinetic system (“PPF1”). The pharmacokinetic system disclosed herein may determine the likelihood by estimating a clearance rate of the biologic drug in a subject. In some embodiments, the clearance rate is determined based, at least in part, on the weight or BMI, level of albumin, and/or CRP. In some embodiments, the PPF is a genetic system factor (“PPF2”). The genetics system disclosed herein may determine whether the subject is at increased risk for developing antibodies against the biologic drug (e.g., autoantibodies) based on the subject's genotype at rs2097432 or a SNV in linkage disequilibrium therewith. In some embodiments, the PPF is a antigenic load system (“PPF3”). In some embodiments the antigenic load system determines whether the subject is likely to respond to the biologic drug based on the level or presence/absence of one or more biomarkers including c-reactive protein (CRP), and/or antibodies against perinuclear antineutrophil cytoplasmic antibodies (pANCA), proteinase 3 (PR3), or certain integrins associated with inflammatory bowel disease (IBD). In some embodiments, the biomarkers comprise (i) a level of CRP, (ii) a presence or absence of pANCA, (iii) a level of PR3, and/or (iv) a level of one or more antibodies against: α4β1, αE, α4, β7, β8, ITGAL, ICAM1, any dimers thereof, or any combinations thereof. In some embodiments, the PPF is a patient wellbeing system (“PPF4”). In some embodiments, the patient wellbeing system may determine a wellbeing status of the subject, based at least in part, on patient wellbeing data received by the patient wellbeing system from the subject. The patient wellbeing data may be information related to symptoms that the subject has been experiencing, or a disease activity score, such as CDAI or Mayo. In some embodiments, the combined PPF model comprises two, three, or four of PPF1, PPF2, PPF3, and PPF4.

In some embodiments, one or more of the statistical models in the patient-centric precision model employs Bayesian assimilation utilizing a set of parameter estimates from a reference population and then inputting subject-specific parameters (e.g., weight, BMI, analyte levels, biomarker levels, patient wellbeing data) to predict disease remission in the subject. The reference population may comprise a population that has received the biologic drug for treatment of the same disease as the subject. In some embodiments, the disease is an immune-mediated inflammatory disease, such as inflammatory bowel disease. In some embodiments, the reference population was non-responsive to the biologic drug, or experienced loss-of-response to the biologic drug. in some embodiments, the reference subjects achieved disease remission. In some embodiments, the reference subject did not achieve disease remission. The subject may not be part of the reference population. In some embodiments, the biologic drug is an anti-TNF therapy.

Using these inputs as previously described herein, a model may be applied to estimate a plurality of conditional distributions of the parameter estimates for the subject. In some embodiments, the parameter estimates are provided in Table 12. In some embodiments, the model may receive input data comprising (i) the analytes or biomarkers detected in the biological sample obtained from the subject, (ii) the weight or BMI of the subject, (iii) patient wellbeing data, or a combination of (i) to (iii). The model may then be interrogated based at least in part based on the data.

In some embodiments, the patient-centric precision model as described herein may determine a biologic drug profile of a biologic drug for a subject having a disease. The biologic drug profile may comprise a dose of the biologic drug at an inter-dose interval estimated to achieve a disease remission in the subject. An algorithm for such model may be trained by receiving data from a database. The data may be related to a pharmacokinetic performance of the biologic drug in individuals from a reference population having the disease that have been treated with the biologic drug. The data may comprise a level of one or more analytes or biomarkers such as those described herein, weight, body mass index (BMI), patient wellbeing data, or any combination thereof. The database may comprise a database for storing electronic medical records (EMRs). In some embodiments, the algorithm may then establish a first set of parameter estimates from the data and further derive a second set of parameter estimates for a model based at least in part on the first set of parameter estimates, as previously described herein.

The model may then receive subject specific data related to the pharmacokinetic performance of the biologic drug, genotype data or rs2097432 or a SNV in linkage disequilibrium therewith, antigenic load, or patient wellbeing data, or any combination thereof. The subject specific data may be obtained by inputting the data into a mobile application on the subject's personal electronic device, such as those described herein. The subject specific data may comprise a level of one or more analytes such as those described herein, weight, BMI, or any combination thereof. The patient wellbeing data comprises a severity of the disease or a symptom thereof, such as, a disease remission, a disease recurrence, a disease type, a frequency of the symptom, a type of the symptom, or any combination thereof. Various non-limiting examples of symptoms of diseases are provided herein. In some embodiments, a severity of the disease or a symptom thereof may be based at least in part of an index or score. The index or score may be adjusted based at least in part on the disease, the model, the subject specific data, the reference population, or any combination thereof. In some embodiments, the index comprises a clinical disease activity index (CDAI). The subject specific data may further comprise weight, BMI, or both. In some embodiments, the subject specific data is contained in one or more electronic medical records (EMRs). The model may be updated based at least in part on the subject specific data that was received. The model may then determine a biologic drug profile for the subject.

The model trained using methods of the present disclosure may be initialized using data received from a reference population that were or currently are being treated with the biologic drug for treatment of an immune-mediated inflammatory disease of the subject. Subject specific parameters may be generated and disease remission for the subject may be simulated based on the subject specific parameters and the data from the reference population. In some embodiments, the subject specific parameters are provided in Table 12. In some embodiments, the model may be updated based on newly received data from the subject, newly received data from the reference population, or both. The model may then estimate a dose of the biologic drug at an inter-dose interval to achieve disease remission in the subject. In some embodiments, the model may estimate that the subject does not achieve remission with the biologic drug.

In some embodiments, the model estimates the dose of the biologic drug at the inter-dose interval with greater than a 50% confidence. In some embodiments, the model estimates the dose of the biologic drug at the inter-dose interval with between about 50% and 95% confidence. In some embodiments, the model estimates the dose of the biologic drug at the inter-dose interval with a confidence comprising about 50% to 95%, 55% to 90%, 60% to 85%, 65% to 80%, or 70% to 75% confidence. In some embodiments, the model estimates the dose of the biologic drug at the inter-dose interval with a confidence comprising about 50%, 55%, 60%, 65%, 70%, 75%, 80%, 85%, 90%, or 95%. In some embodiments, the model estimates the dose of the biologic drug at the inter-dose interval with a confidence of at least about 50%, 55%, 60%, 65%, 70%, 75%, 80%, 85%, 90%, or 95%. In some embodiments, the model estimates the dose of the biologic drug at the inter-dose interval with greater than or equal to about a 90% confidence.

In some embodiments, the model estimates that the subject will not achieve disease remission with the biologic drug (e.g., anti-TNF therapy) with a confidence comprising about 50% to 95%. In some embodiments, the confidence comprises about 50% to 95%, 55% to 90%, 60% to 85%, 65% to 80%, or 70% to 75%. In some embodiments, the confidence comprises about 50%, 55%, 60%, 65%, 70%, 75%, 80%, 85%, 90%, 91%, 92%, 93%, 94%, or 95%. In some embodiments, the confidence is at least about 50%, 55%, 60%, 65%, 70%, 75%, 80%, 85%, 90%, 91%, 92%, 93%, 94%, or 95%. In some embodiments, the confidence is at least about 90%.

In some embodiments, the model estimates the dose of the biologic drug at the inter-dose interval of the biologic drug (e.g., anti-TNF therapy) to achieve disease remission with a specificity comprising about 50% to 95%. In some embodiments, the specificity comprises about 50% to 95%, 55% to 90%, 60% to 85%, 65% to 80%, or 70% to 75%. In some embodiments, the specificity comprises about 50%, 55%, 60%, 65%, 70%, 75%, 80%, 85%, 90%, 91%, 92%, 93%, 94%, or 95%. In some embodiments, the specificity is at least about 50%, 55%, 60%, 65%, 70%, 75%, 80%, 85%, 90%, 91%, 92%, 93%, 94%, or 95%. In some embodiments, the specificity is at least about 90%.

In some embodiments, the model estimates that the subject will not achieve disease remission with the biologic drug (e.g., anti-TNF therapy) with a specificity comprising about 50% to 95%. In some embodiments, the specificity comprises about 50% to 95%, 55% to 90%, 60% to 85%, 65% to 80%, or 70% to 75%. In some embodiments, the specificity comprises about 50%, 55%, 60%, 65%, 70%, 75%, 80%, 85%, 90%, 91%, 92%, 93%, 94%, or 95%. In some embodiments, the specificity is at least about 50%, 55%, 60%, 65%, 70%, 75%, 80%, 85%, 90%, 91%, 92%, 93%, 94%, or 95%. In some embodiments, the specificity is at least about 90%.

In some embodiments, the model estimates the dose of the biologic drug at the inter-dose interval of the biologic drug (e.g., anti-TNF therapy) to achieve disease remission with a positive predictive value comprising about 50% to 95%. In some embodiments, the positive predictive value comprises about 50% to 95%, 55% to 90%, 60% to 85%, 65% to 80%, or 70% to 75%. In some embodiments, the positive predictive value comprises about 50%, 55%, 60%, 65%, 70%, 75%, 80%, 85%, 90%, 91%, 92%, 93%, 94%, or 95%. In some embodiments, the positive predictive value is at least about 50%, 55%, 60%, 65%, 70%, 75%, 80%, 85%, 90%, 91%, 92%, 93%, 94%, or 95%. In some embodiments, the positive predictive value is at least about 90%.

In some embodiments, the model estimates that the subject will not achieve disease remission with the biologic drug (e.g., anti-TNF therapy) with a positive predictive value comprising about 50% to 95%. In some embodiments, the positive predictive value comprises about 50% to 95%, 55% to 90%, 60% to 85%, 65% to 80%, or 70% to 75%. In some embodiments, the positive predictive value comprises about 50%, 55%, 60%, 65%, 70%, 75%, 80%, 85%, 90%, 91%, 92%, 93%, 94%, or 95%. In some embodiments, the positive predictive value is at least about 50%, 55%, 60%, 65%, 70%, 75%, 80%, 85%, 90%, 91%, 92%, 93%, 94%, or 95%. In some embodiments, the positive predictive value is at least about 90%.

In some embodiments, the model estimates the dose of the biologic drug at the inter-dose interval of the biologic drug (e.g., anti-TNF therapy) to achieve disease remission with a negative predictive value comprising about 50% to 95%. In some embodiments, the negative predictive value comprises about 50% to 95%, 55% to 90%, 60% to 85%, 65% to 80%, or 70% to 75%. In some embodiments, the negative predictive value comprises about 50%, 55%, 60%, 65%, 70%, 75%, 80%, 85%, 90%, 91%, 92%, 93%, 94%, or 95%. In some embodiments, the negative predictive value is at least about 50%, 55%, 60%, 65%, 70%, 75%, 80%, 85%, 90%, 91%, 92%, 93%, 94%, or 95%. In some embodiments, the negative predictive value is at least about 90%.

In some embodiments, the model estimates that the subject will not achieve disease remission with the biologic drug (e.g., anti-TNF therapy) with a negative predictive value comprising about 50% to 95%. In some embodiments, the negative predictive value comprises about 50% to 95%, 55% to 90%, 60% to 85%, 65% to 80%, or 70% to 75%. In some embodiments, the negative predictive value comprises about 50%, 55%, 60%, 65%, 70%, 75%, 80%, 85%, 90%, 91%, 92%, 93%, 94%, or 95%. In some embodiments, the negative predictive value is at least about 50%, 55%, 60%, 65%, 70%, 75%, 80%, 85%, 90%, 91%, 92%, 93%, 94%, or 95%. In some embodiments, the negative predictive value is at least about 90%.

In some embodiments, the model estimates the dose of the biologic drug at the inter-dose interval of the biologic drug (e.g., anti-TNF therapy) to achieve disease remission with an area under the curve (AUC) comprising about 0.50 to 0.90. In some embodiments, the area under the curve (AUC) comprises about 0.50 to 0.095, 0.55 to 0.90, 0.60 to 0.85, 0.65 to 0.80, or 0.70 to 0.75. In some embodiments, the area under the curve (AUC) comprises about 0.50, 0.55, 0.60, 0.65, 0.70, 0.75, 0.80, 0.85, 0.90, 0.91, 0.92, 0.93, 0.94, or 0.95. In some embodiments, the area under the curve (AUC) is at least about 0.50, 055, 0.60, 0.65, 0.70, 0.75, 0.80, 0.85, 0.90, 0.91, 0.92, 0.93, 0.94, or 0.95. In some embodiments, the area under the curve (AUC) is at least about 0.90.

In some embodiments, the model estimates that the subject will not achieve disease remission with the biologic drug (e.g., anti-TNF therapy) with an area under the curve (AUC) comprising about 0.50 to 0.90. In some embodiments, the area under the curve (AUC) comprises about 0.50 to 0.095, 0.55 to 0.90, 0.60 to 0.85, 0.65 to 0.80, or 0.70 to 0.75. In some embodiments, the area under the curve (AUC) comprises about 0.50, 0.55, 0.60, 0.65, 0.70, 0.75, 0.80, 0.85, 0.90, 0.91, 0.92, 0.93, 0.94, or 0.95. In some embodiments, the area under the curve (AUC) is at least about 0.50, 055, 0.60, 0.65, 0.70, 0.75, 0.80, 0.85, 0.90, 0.91, 0.92, 0.93, 0.94, or 0.95. In some embodiments, the area under the curve (AUC) is at least about 0.90.

In some embodiments, if the model estimates that the subject will achieve disease remission with the biologic drug that is over the maximum dose for that drug, then the model may indicate that the subject should be treated with another therapeutic agent disclosed herein. In some embodiments, the another therapeutic agent may be a small molecule inhibitor. In some embodiments, the small molecule inhibitor is specific to a Janus Kinase (JAK). In some embodiments, the small molecule inhibitor specific to JAK comprises baricitinib, tofacitinib, or upadacitinib, or any combination thereof. In some embodiments, the small molecule is specific to a sphingosine 1-phosphate (S1P) modulator or an S1P receptor modulator. In some embodiments, the small molecule specific to an S1P receptor modulator comprises fingolimod, siponimod, ozanimod, or ponesimod, or any combination thereof. In some embodiments, the biologic drug is an anti-TNF therapy. In some embodiments, the anti-TNF therapy is or comprises Infliximab, Adalimumab, Eetanercept, Golimumab, or Certolizumab, or any combination thereof. In some embodiments, the biologic drug may be an inhibitor of a407 integrin (e.g., Vedolizumab). In some embodiments, the another therapeutic agent may be an inhibitor of interleukin 12 (IL-12) or interleukin 23 (IL-23) activity or signaling. In some embodiments, the inhibitor of IL-12 of IL-23 is or comprises Ustekinumab.

### Input Device

In some embodiments, the computing system comprises a input device. The input device may be used to input user information that can be accessed on the computing system. The input device may further be used to receive subject specific information, which can be accessed on the computing system. In some embodiments, the subject of the subject specific information has a disease (e.g., cancer, an immune-mediated inflammatory disease, etc.). User information may comprise symptoms, symptom severity, symptom duration, weight, temperature, heart rate, etc. In some embodiments, user information is obtained from a survey displayed on an input device. In some embodiments, the input device comprises a sensor. In some embodiments, the sensor may be used to track a user's health. A sensor may include, but is not limited to, an accelerometer, a heart rate sensor, a blood pressure sensor, a blood glucose sensor, a sweat sensor a skin conductivity sensor or an imaging sensor, or a spectrometer. In some embodiments, the input device comprises a communication element (e.g., Bluetooth®) configured to transmit the recorded sensor data to the computing system.

In some embodiments, the input device comprises a personal electronic device. In some embodiments, the personal electronic device is a handheld device. A handheld device may comprise, by way of non-limiting example, a mobile device, audio device, tablet, laptop, or any of various other mobile computing device. In some embodiments, the personal electronic device is an embedded device (e.g., a glucose monitor, a pacer, etc.). In some embodiments, the personal electronic device is worn (e.g., accessories, clothing, etc.). In some embodiments, the personal electronic device is a health tracking device. A health tracking device may comprise, by way of non-limiting example, a Fitbit®, Amazfit®, Oura Ring®, Garmin®, Apple Watch®, Galaxy Watch®, Whoop®, Jawbone®, Polar®, Under Armour® etc.

In some embodiments, the input device comprises an interface comprising a software and hardware configured to facilitate communications with the input device and the user. The hardware may comprise a display screen configured to display graphics, texts, or any other visual data. In some embodiments, the display screen is a touch screen. In some embodiments, the display screen comprises a virtual keyboard for inputting information. In alternative embodiments, a physical keyboard is used to input information. The hardware may further comprise a microphone and/or speakers to facilitate audio communications with the user. The software may comprise an application, such as those described herein. The software may be configured for a user to input information, such as clinical data or health data (e.g., symptom severity, weight, etc.). An non-limiting example of one or more user interfaces of a mobile device is provided in FIG. 8.

### Non-Transitory Computer Readable Storage Medium

In some embodiments, the platforms, systems, media, and methods disclosed herein include one or more non-transitory computer readable storage media encoded with a program including instructions executable by the operating system of an optionally networked computing device. In further embodiments, a computer readable storage medium is a tangible component of a computing device. In still further embodiments, a computer readable storage medium is optionally removable from a computing device. In some embodiments, a computer readable storage medium includes, by way of non-limiting examples, CD-ROMs, DVDs, flash memory devices, solid state memory, magnetic disk drives, magnetic tape drives, optical disk drives, distributed computing systems including cloud computing systems and services, and the like. In some cases, the program and instructions are permanently, substantially permanently, semi-permanently, or non-transitorily encoded on the media.

In some embodiments, the non-transitory computer-readable storage media is encoded with a computer program including instructions executable by one or more processors for predicting disease remission in a subject, based on pharmacokinetic performance of the biologic drug (e.g., anti-TNF therapy). In some embodiments, the instructions comprise: a) initializing a model of a biologic drug concentration profile for a biologic drug, b) generating subject specific parameters relating to pharmacokinetic performance of the biologic drug in the subject; c) simulating the biologic drug concentration profile for the subject based on the subject specific parameters and the data from the reference population; d) updating the model based on newly received data from the subject in (b) and newly received data from the reference population in (a); and e) estimating a dose of the biologic drug at an inter-dose interval to achieve disease remission in the subject with the model updated in (d). In some embodiments, the model comprises data received from a reference population that were or currently are being treated with the biologic drug for treatment of an immune-mediated inflammatory disease. In some embodiments, the threshold biologic drug concentration is sufficient to treat the immune-mediated inflammatory disease in the subject.

In some embodiments, the non-transitory computer-readable storage media is encoded with a computer program including instructions executable by one or more processors for predicting disease remission in a subject, based on pharmacokinetic performance of the biologic drug (e.g., anti-TNF therapy). In some embodiments, the instructions comprise: a) initializing a model of a time to disease remission for a subject, b) generating subject specific parameters relating to pharmacokinetic performance of the biologic drug in the subject, the subject's genetic risk for developing autoantibodies against the biologic drug, the antigenic load measured in a biological sample obtained from the subject, and/or the subject's wellbeing status; c) simulating the time to remission profile for the subject based on the subject specific parameters and the data from the reference population; d) updating the model based on newly received data from the subject in (b) and newly received data from the reference population in (a); and e) estimating the time to remission in the subject with the model updated in (d). In some embodiments, the model comprises data received from a reference population that were or currently are being treated with the biologic drug for treatment of an immune-mediated inflammatory disease. In some embodiments, the biologic drug is an anti-TNF therapy. In some embodiments, the anti-TNF therapy is or comprises Infliximab, Adalimumab, Eetanercept, Golimumab, or Certolizumab, or any combination thereof. In some embodiments, the biologic drug may be an inhibitor of a407 integrin (e.g., Vedolizumab). In some embodiments, the another therapeutic agent may be an inhibitor of interleukin 12 (IL-12) or interleukin 23 (IL-23) activity or signaling. In some embodiments, the inhibitor of IL-12 of IL-23 is or comprises Ustekinumab.

In some embodiments, the non-transitory computer-readable storage media is encoded with a computer program including instructions executable by one or more processors for predicting disease remission in a subject, based on genetic risk (e.g., PPF2). In some embodiments, the instructions comprise: a) initializing a model of a genetic risk profile, b) generating subject specific parameters relating to the subject's genotype at rs2097432 or a SNV in linkage disequilibrium therewith; c) simulating the genetic risk profile for the subject based on the subject specific parameters and the data from the reference population; d) updating the model based on newly received data from the subject in (b) and newly received data from the reference population in (a); and e) estimating time to disease remission in the subject with the model updated in (d). In some embodiments, the model comprises data received from a reference population that were or currently are being treated with the biologic drug for treatment of an immune-mediated inflammatory disease. In some embodiments, the threshold biologic drug concentration is sufficient to treat the immune-mediated inflammatory disease in the subject.

In some embodiments, the non-transitory computer-readable storage media is encoded with a computer program including instructions executable by one or more processors for predicting disease remission in a subject, based on patient wellbeing status. In some embodiments, the instructions comprise: a) initializing a model of patient wellbeing profile, b) generating subject specific parameters relating to the subject's wellbeing; c) simulating the patient wellbeing profile for the subject based on the subject specific parameters and the data from the reference population; d) updating the model based on newly received data from the subject in (b) and newly received data from the reference population in (a); and e) estimating time to disease remission in the subject with the model updated in (d). In some embodiments, the model comprises data received from a reference population that were or currently are being treated with the biologic drug for treatment of an immune-mediated inflammatory disease. In some embodiments, the threshold biologic drug concentration is sufficient to treat the immune-mediated inflammatory disease in the subject. In some embodiments, the subject-specific parameters include information related to the subject's symptoms or disease activity.

In some embodiments, the non-transitory computer-readable storage media is encoded with a computer program including instructions executable by one or more processors to calculate a combined poor prognosis factor (PPF) score for a subject using a patient-centric precision model, comprising: (a) a first software module configured to calculate a poor prognosis factor 1 (PPF1) score for the subject comprising: (i) receiving a level of albumin from a biological sample obtained from a subject, and a weight of the subject; (ii) calculating a rate of which a biologic drug is estimated to be cleared from the subject using a first statistical model based, at least in part, on the level of the albumin measured in the biological sample, and the weight of the subject; and (iii) comparing the rate to a predetermined threshold for the biologic drug, to produce an estimated clearance score on a scale of 0 to 1, wherein 0 is indicative of a low rate of clearance below the predetermined threshold, and 1 is indicative of a high rate of clearance above the predetermined threshold; (b) a second software module configured to calculate a poor prognosis factor 2 (PPF 2) score for the subject comprising: (i) receiving a genotype of the subject at single nucleotide variant (SNV) comprising rs2097432 or a SNV in linkage disequilibrium therewith; and (ii) calculating a PPF2 score using a second statistical model, wherein the PPF2 score is on a scale of 0 to 2, wherein 0 is indicative of the genotype that is homozygous non-risk, 1 is indicative of the genotype that is heterozygous risk at rs2097432, and 2 comprises a presence of two genetic loci of said genotype; and (c) a third software module configured to calculate a poor prognosis factor 3 (PPF3) score or the subject, comprising: (i) calculating a PPF3 score using a third statistical model based on a presence or a level of one or more serological markers detected in a biological sample obtained from the subject, wherein 0 is indicative of a standard response to the biologic drug, 1 is indicative of a low response to the biologic drug, and 2 is indicative of very low response to the biologic drug; and (c) a forth software module configured to calculate a poor prognosis factor 4 (PPF 4) for the subject comprising: (i) receiving wellbeing data from said subject comprising one or more symptoms related to the immune-mediated inflammatory disease; (ii) calculating a wellbeing score with a fourth statistical model of 0 or 1 based on the wellbeing data received, wherein 0 is indicative of an improvement of at least one symptom of the one or more symptoms, and 1 is indicative of no improvement in the at least one symptom of the one or more symptoms; and (d) a fifth software module configured to calculate a combined PPF score comprising at least two of the PPF1 score, the PPF2 score, the PPF3 score, and the PPF4 score to predict a time to remission following administration of the biologic drug to the subject. In some embodiments, the weight of the subject and the level of albumin values are log transformed and divided by the typical value from a reference population. In some embodiments, the typical value for weight and level of albumin is an average (mean) value or a median value. In some embodiments, the reference population contains reference individuals with the same disease or condition as the subject that have received a biologic drug to treat the disease or the condition.

### Computer Program

In some embodiments, the platforms, systems, media, and methods disclosed herein include at least one computer program, or use of the same. A computer program includes a sequence of instructions, executable by one or more processor(s) of the computing device's CPU, written to perform a specified task. As such, the computer programs disclosed herein may include a sequence of instructions to perform one or more method disclosed herein. Computer readable instructions may be implemented as program modules, such as functions, objects, Application Programming Interfaces (APIs), computing data structures, and the like, that perform particular tasks or implement particular abstract data types. In light of the disclosure provided herein, those of skill in the art will recognize that a computer program may be written in various versions of various languages.

The functionality of the computer readable instructions may be combined or distributed as desired in various environments. In some embodiments, a computer program comprises one sequence of instructions. In some embodiments, a computer program comprises a plurality of sequences of instructions. In some embodiments, a computer program is provided from one location. In other embodiments, a computer program is provided from a plurality of locations. In various embodiments, a computer program includes one or more software modules. In various embodiments, a computer program includes, in part or in whole, one or more web applications, one or more mobile applications, one or more standalone applications, one or more web browser plug-ins, extensions, add-ins, or add-ons, or combinations thereof.

Disclosed herein, in some embodiments, are computer programs including a sequence of instructions comprising: (a) calculating an estimated clearance rate at which an anti-tumor necrosis factor (TNF) therapy is cleared from the subject using a first statistical model based, at least in part, on a level of albumin measured in a biological sample obtained from the subject, and a weight of the subject; (b) comparing the clearance rate calculated in (a) to a predetermined threshold, to produce poor prognosis factor 1 (PPF1) score on a scale of 0 to 1, wherein 0 is indicative of a low rate of clearance below the predetermined threshold, and 1 is indicative of a high rate of clearance above the predetermined threshold; (c) calculating a poor prognosis factor 2 (PPF2) score on a scale of 0 to 2 with a second statistical model, based at least in part, on a presence of a genotype at a single nucleotide variant (SNV) comprising rs2097432 or a SNV in linkage disequilibrium therewith detected in the biological sample obtained from the subject, wherein 0 is indicative of the genotype that is homozygous non-risk, 1 is indicative of the genotype that is heterozygous risk at rs2097432, and 2 comprises a presence of two genetic loci of said genotype; (d) calculating a poor prognosis factor 3 (PPF3) score on a scale of 0 to 2 with a third statistic model based on the presence or level of one or more serological markers detected in a biological sample obtained from the subject, wherein 0 is indicative of a standard response to the biologic drug, 1 is indicative e of a low response to the biologic drug, and 2 is indicative of very low response to the biologic drug; (e) calculating a poor prognosis factor 4 (PPF4) score on a scale of 0 to 1 with a third statistical model, based at least in part on patient wellbeing received from the subject, wherein a PPF4 score of 0 is indicative of an improvement of at least one symptom of the immune-mediated inflammatory disease, and a PPF4 score of 1 is indicative of no improvement in the at least one symptom; and calculating a combined PPF score comprising the PPF1 score, the PPF2 score, the PPF3 score and the PPF4 score, to predict a time to remission for the subject. In some embodiments, the time to remission is reflected in a percentage reduction in hazard remission. In some embodiments, the percentage reduction in hazard remission above 78% indicates that the subject is suitable for treatment with the biologic drug at a first dose. In some embodiments, the percentage reduction in hazard remission comprising 56% to 78% indicates that the subject is suitable for treatment with the biologic drug at a second dose that is higher than the first dose. In some embodiments, the percentage reduction in hazard remission comprising 34% to 55% indicates that the subject is suitable for treatment with the biologic drug at a third dose that is higher than the second dose and the first dose. In some embodiments, a percentage reduction in hazard remission below 56% indicates that the subject is not suitable for treatment with the biologic drug, and should be prescribed a different therapeutic agent, such as a small molecule inhibitor. For example, if the biologic drug is infliximab, the first dose comprises 5 milligrams per kilogram (mg/Kg) and the second dose comprises 7.5 mg/Kg, and the third dose comprises 10 mg/Kg.

### Web Application

In some embodiments, a computer program includes a web application. In light of the disclosure provided herein, those of skill in the art will recognize that a web application, in various embodiments, utilizes one or more software frameworks and one or more database systems. In some embodiments, a web application is created upon a software framework such as Microsoft® .NET or Ruby on Rails (RoR). In some embodiments, a web application utilizes one or more database systems including, by way of non-limiting examples, relational, non-relational, object oriented, associative, XML, and document oriented database systems. In further embodiments, suitable relational database systems include, by way of non-limiting examples, Microsoft® SQL Server, mySQL™, and Oracle®. Those of skill in the art will also recognize that a web application, in various embodiments, is written in one or more versions of one or more languages. A web application may be written in one or more markup languages, presentation definition languages, client-side scripting languages, server-side coding languages, database query languages, or combinations thereof. In some embodiments, a web application is written to some extent in a markup language such as Hypertext Markup Language (HTTML), Extensible Hypertext Markup Language (XHTML), or eXtensible Markup Language (XML). In some embodiments, a web application is written to some extent in a presentation definition language such as Cascading Style Sheets (CSS). In some embodiments, a web application is written to some extent in a client-side scripting language such as Asynchronous JavaScript and XML (AJAX), Flash® ActionScript, JavaScript, or Silverlight®. In some embodiments, a web application is written to some extent in a server-side coding language such as Active Server Pages (ASP), ColdFusion®, Perl, Java™ JavaServer Pages (JSP), Hypertext Preprocessor (PHP), Python™, Ruby, Tcl, Smalltalk, WebDNA®, or Groovy. In some embodiments, a web application is written to some extent in a database query language such as Structured Query Language (SQL). In some embodiments, a web application integrates enterprise server products such as IBM® Lotus Domino®. In some embodiments, a web application includes a media player element. In various further embodiments, a media player element utilizes one or more of many suitable multimedia technologies including, by way of non-limiting examples, Adobe® Flash®, HTML 5, Apple® QuickTime®, Microsoft® Silverlight®, Java™, and Unity®.

Referring to FIG. 34, in a particular embodiment, an application provision system comprises one or more databases 200 accessed by a relational database management system (RDBMS) 210. Suitable RDBMSs include Firebird, My SQL, PostgreSQL, SQLite, Oracle Database, Microsoft SQL Server, IBM DB2, IBM Informix, SAP Sybase, Teradata, and the like. In this embodiment, the application provision system further comprises one or more application severs 220 (such as Java servers, NET servers, PUP servers, and the like) and one or more web servers 230 (such as Apache, IIS, GWS and the like). The web server(s) optionally expose one or more web services via app application programming interfaces (APIs) 240. Via a network, such as the Internet, the system provides browser-based and/or mobile native user interfaces.

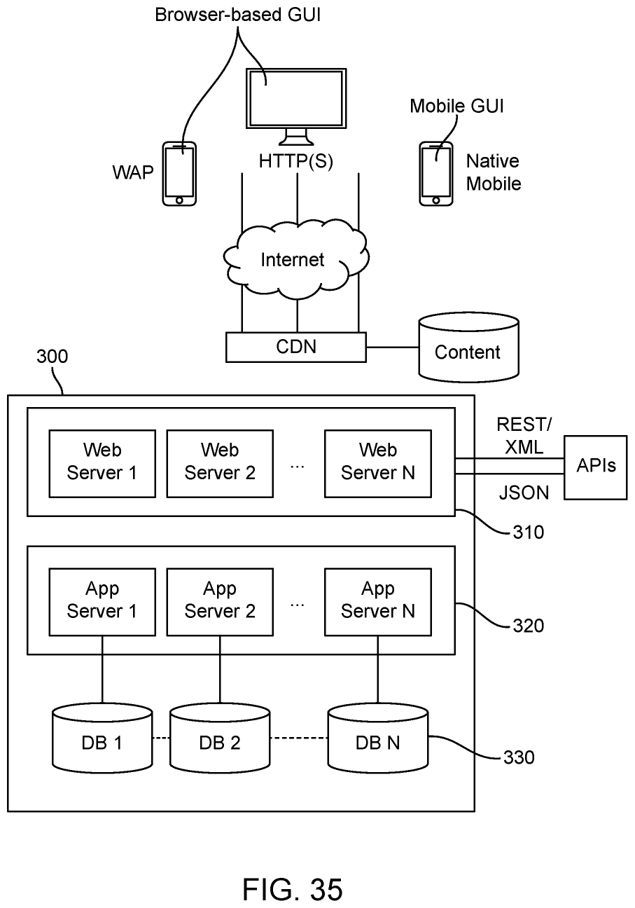

Referring to FIG. 35, in a particular embodiment, an application provision system alternatively has a distributed, cloud-based architecture 300 and comprises elastically load balanced, auto-scaling web server resources 310 and application server resources 320 as well synchronously replicated databases 330.

### Mobile Application

In some embodiments, a computer program includes a mobile application provided to a mobile computing device. In some embodiments, the mobile application is provided to a mobile computing device at the time it is manufactured. In other embodiments, the mobile application is provided to a mobile computing device via the computer network described herein.

In some embodiments, the mobile application comprises an application for tracking or logging patient health. In some embodiments, the mobile application may comprise an interface, such as, for example, the interface shown in FIG. 8. In some embodiments, the mobile application tracks a patient's symptoms of a disease, severity of a disease, diet, exercise, medications, sensor data (e.g., heart rate, glucose levels, etc.), or any other relevant patient information. In some embodiments, the mobile application comprises a survey for assessing patient wellness, such as but not limited to, those described herein. In some embodiments, the mobile application may be provided as a web application, a standalone application, or both.

In view of the disclosure provided herein, a mobile application is created by techniques known to those of skill in the art using hardware, languages, and development environments known to the art. Those of skill in the art will recognize that mobile applications are written in several languages. Suitable programming languages include, by way of non-limiting examples, C, C++, C#, Objective-C, Java™, JavaScript, Pascal, Object Pascal, Python™, Ruby, VB.NET, WML, and XHTML/HTML with or without CSS, or combinations thereof.

Suitable mobile application development environments are available from several sources. Commercially available development environments include, by way of non-limiting examples, AirplaySDK, alcheMo, Appcelerator®, Celsius, Bedrock, Flash Lite, .NET Compact Framework, Rhomobile, and WorkLight Mobile Platform. Other development environments are available without cost including, by way of non-limiting examples, Lazarus, MobiFlex, MoSync, and Phonegap. Also, mobile device manufacturers distribute software developer kits including, by way of non-limiting examples, iPhone and iPad (iOS) SDK, Android™ SDK, BlackBerry® SDK, BREW SDK, Palm® OS SDK, Symbian SDK, webOS SDK, and Windows® Mobile SDK.

Those of skill in the art will recognize that several commercial forums are available for distribution of mobile applications including, by way of non-limiting examples, Apple® App Store, Google® Play, Chrome Web Store, BlackBerry® App World, App Store for Palm devices, App Catalog for webOS, Windows® Marketplace for Mobile, Ovi Store for Nokia® devices, Samsung® Apps, and Nintendo® DSi Shop.

### Standalone Application

In some embodiments, a computer program includes a standalone application, which is a program that is run as an independent computer process, not an add-on to an existing process, e.g., not a plug-in. In some embodiments, the standalone application comprises the same features for tracking or logging patient health as a mobile application, as previously described herein. Those of skill in the art will recognize that standalone applications are often compiled. A compiler is a computer program(s) that transforms source code written in a programming language into binary object code such as assembly language or machine code. Suitable compiled programming languages include, by way of non-limiting examples, C, C++, Objective-C, COBOL, Delphi, Eiffel, Java™, Lisp, Python™, Visual Basic, and VB .NET, or combinations thereof. Compilation is often performed, at least in part, to create an executable program. In some embodiments, a computer program includes one or more executable complied applications.

### Web Browser Plug-In

In some embodiments, the computer program includes a web browser plug-in (e.g., extension, etc.). In computing, a plug-in is one or more software components that add specific functionality to a larger software application. Makers of software applications support plug-ins to enable third-party developers to create abilities which extend an application, to support easily adding new features, and to reduce the size of an application. When supported, plug-ins enable customizing the functionality of a software application. For example, plug-ins are commonly used in web browsers to play video, generate interactivity, scan for viruses, and display particular file types. Those of skill in the art will be familiar with several web browser plug-ins including, Adobe® Flash® Player, Microsoft® Silverlight®, and Apple® QuickTime®. In some embodiments, the toolbar comprises one or more web browser extensions, add-ins, or add-ons. In some embodiments, the toolbar comprises one or more explorer bars, tool bands, or desk bands.

In view of the disclosure provided herein, those of skill in the art will recognize that several plug-in frameworks are available that enable development of plug-ins in various programming languages, including, by way of non-limiting examples, C++, Delphi, Java™ PHP, Python™, and VB NET, or combinations thereof.

Web browsers (also called Internet browsers) are software applications, designed for use with network-connected computing devices, for retrieving, presenting, and traversing information resources on the World Wide Web. Suitable web browsers include, by way of non-limiting examples, Microsoft® Internet Explorer®, Mozilla® Firefox®, Google® Chrome, Apple® Safari®, Opera Software® Opera®, and KDE Konqueror. In some embodiments, the web browser is a mobile web browser. Mobile web browsers (also called microbrowsers, mini-browsers, and wireless browsers) are designed for use on mobile computing devices including, by way of non-limiting examples, handheld computers, tablet computers, netbook computers, subnotebook computers, smartphones, music players, personal digital assistants (PDAs), and handheld video game systems. Suitable mobile web browsers include, byway of non-limiting examples, Google® Android® browser, RIM BlackBerry® Browser, Apple® Safari®, Palm® Blazer, Palm® WebOS® Browser, Mozilla® Firefox® for mobile, Microsoft® Internet Explorer® Mobile, Amazon® Kindle® Basic Web, Nokia® Browser, Opera Software® Opera® Mobile, and Sony® PSP™ browser.

### Software Modules

In some embodiments, the platforms, systems, media, and methods disclosed herein include software, server, and/or database modules, or use of the same. In view of the disclosure provided herein, software modules are created by techniques known to those of skill in the art using machines, software, and languages known to the art. The software modules disclosed herein are implemented in a multitude of ways. In various embodiments, a software module comprises a file, a section of code, a programming object, a programming structure, a distributed computing resource, a cloud computing resource, or combinations thereof. In further various embodiments, a software module comprises a plurality of files, a plurality of sections of code, a plurality of programming objects, a plurality of programming structures, a plurality of distributed computing resources, a plurality of cloud computing resources, or combinations thereof. In various embodiments, the one or more software modules comprise, by way of non-limiting examples, a web application, a mobile application, a standalone application, and a distributed or cloud computing application. In some embodiments, software modules are in one computer program or application. In other embodiments, software modules are in more than one computer program or application. In some embodiments, software modules are hosted on one machine. In other embodiments, software modules are hosted on more than one machine. In further embodiments, software modules are hosted on a distributed computing platform such as a cloud computing platform. In some embodiments, software modules are hosted on one or more machines in one location. In other embodiments, software modules are hosted on one or more machines in more than one location.

In some embodiments, an application described herein comprises one or more software modules. In some embodiments, a software module is configured to initialize a model of a biologic drug concentration profile for a biologic drug. In some embodiments, the model comprises data related to a pharmacokinetic performance of the biologic drug in individuals from a reference population, as described herein. In some embodiments, the model is a statistical model, a numerical model, a machine learning model, or any combination thereof. In some embodiments, the reference population has a disease that have been treated with the biologic drug. In some embodiments, the disease is an immune-mediated inflammatory disease. In some embodiments, the disease is cancer. In some embodiments, a software module is configured to establish a first set of parameter estimates from the data. In some embodiments, a software module is configured to derive a second set of parameter estimates for a model based at least in part on the first set of parameter estimates. In some embodiments, a software module is configured to receive subject specific data related to the pharmacokinetic performance of the biologic drug in the subject. In some embodiments, the subject specific data is received using an input device, such as those described herein. In some embodiments, a software module is configured to update the model based at least in part on the subject specific data. In some embodiments, a software module is configured to determine a biologic drug profile for the subject. In some embodiments, the biologic drug profile comprises a dose of the biologic drug at an inter-dose interval estimated to achieve a threshold biologic drug concentration value in the subject that is sufficient to treat the disease in the subject.

In some embodiments the patient-centric precision model comprises one or more software modules. In some embodiments, the one or more software modules comprises (a) a first software module configured to calculate a poor prognosis factor 1 (PPF1) score for the subject comprising: (i) receiving a level of albumin from a biological sample obtained from a subject, and a weight of the subject; (ii) calculating a rate of which biologic drug is estimated to be cleared from the subject using a first statistical model based, at least in part, on the level of the albumin measured in the biological sample and the weight of the subject; and (iii) comparing the rate to a predetermined threshold for the biologic drug, to produce an estimated clearance score on a scale of 0 to 1, wherein 0 is indicative of a low rate of clearance below the predetermined threshold, and 1 is indicative of a high rate of clearance above the predetermined threshold. In some embodiments, the one or more software modules comprises (b) a second software module configured to calculate a poor prognosis factor 2 (PPF 2) score for the subject comprising: (i) receiving a genotype of the subject at single nucleotide variant (SNV) comprising rs2097432 or a SNV in linkage disequilibrium therewith; and (ii) calculating a PPF2 score using a second statistical model, wherein the PPF2 score is on a scale of 0 to 2, wherein 0 is indicative of the genotype that is homozygous non-risk, 1 is indicative of the genotype that is heterozygous risk at rs2097432, and 2 comprises a presence of two genetic loci of said genotype. In some embodiments, the one or more software modules comprises (c) a third software module configured to calculate a poor prognosis factor 3 (PPF3) on a scale of 0 to 2 using a third statistical model, based on a presence or a level of one or more serological markers (e.g., pANCA, PR3) detected in a biological sample obtained from the subject, wherein 0 is indicative of a standard response to the biologic drug, 1 is indicative e of a low response to the biologic drug, and 2 is indicative of very low response to the biologic drug. In some embodiments, the one or more software modules comprises (d) a forth software module configured to calculate a poor prognosis factor 4 (PPF 4) for the subject comprising: (i) receiving wellbeing data from said subject comprising one or more symptoms related to the immune-mediated inflammatory disease; (ii) calculating a wellbeing score with a fourth statistical model of 0 or 1 based on the wellbeing data received, wherein 0 is indicative of an improvement of at least one symptom of the one or more symptoms, and 1 is indicative of no improvement in the at least one symptom of the one or more symptoms. In some embodiments, the one or more software modules comprises (e) a fifth software module configured to calculate a combined PPF score comprising at least two of the PPF1 score, the PPF2 score, the PPF3 score, and the PPF4 score to predict a time to remission following administration of the biologic drug to the subject. In some embodiments, the fifth software module is configured to calculate the combined PPF score based on all four PPF scores. In some embodiments, the weight of the subject and the level of albumin values are log transformed and divided by the typical value from a reference population. In some embodiments, the typical value for weight and level of albumin is an average (mean) value or a median value. In some embodiments, the reference population contains reference individuals with the same disease or condition as the subject that have received an biologic drug to treat the disease or the condition.

### Databases

In some embodiments, the platforms, systems, media, and methods disclosed herein include one or more databases, or use of the same. In some embodiments, a database comprises user information comprising user health or wellness information. In some embodiments, the user information comprises subject specific data from one or more subject comprising a disease. In some embodiments, the subject specific data comprises symptoms, symptom severity, weight, BMI, CDAI score, survey data, one or more sensor measurements as described herein, one or more analyte concentrations as those described herein, or any combination thereof. In some embodiments, the database comprises electronic medical records (EMRs). In some embodiments, the data may be accessed by a user, such as the subject. In some embodiments, the data may be accessed by a healthcare professional, such as, but not limited to, a physician, nurse, pharmacist, healthcare administrator, therapist, etc.

In view of the disclosure provided herein, those of skill in the art will recognize that many databases are suitable for storage and retrieval of subject information. In various embodiments, suitable databases include, by way of non-limiting examples, relational databases, non-relational databases, object oriented databases, object databases, entity-relationship model databases, associative databases, XML databases, document oriented databases, and graph databases. Further non-limiting examples include SQL, PostgreSQL, MySQL, Oracle, DB2, Sybase, and MongoDB. In some embodiments, a database is Internet-based. In further embodiments, a database is web-based. In still further embodiments, a database is cloud computing-based. In a particular embodiment, a database is a distributed database. In other embodiments, a database is based on one or more local computer storage devices.

### Methods

Disclosed herein, in some embodiments, are methods for estimating the time to disease remission for a subject using the patient-centric precision model disclosed herein. In some embodiments, methods further comprise determining a dosage or type of therapeutic agent to administer to a subject to treat the immune-mediated inflammatory disease disclosed herein (e.g., inflammatory bowel disease) based, at least in part, on a combined PPF score for the subject calculated using the patient-centric precision model disclosed herein. In some embodiments, methods may involve receiving one or more of genetic data, patient wellbeing information, a level or a presence/absence of one or more analytes or biomarkers detected in a biological sample obtained from the subject (e.g., albumin, CRP, PR3, pANCA, etc.), or clinical data (e.g., the subject's age, weight, gender, smoking status, and the like). In some embodiments, the methods utilize the systems disclosed herein to calculate a combined PPF score that is predictive of a time to remission of the immune-mediated inflammatory disease based on the calculated combined PPF score. A percentage reduction in hazard remission may be calculated based on this time to remission, which can be used to determine a type, dose and/or inter-dose interval for biologic drug to be administered to the subject. In some embodiments, if the dose or inter-dose interval of the biologic drug is predicted to exceed the approved dosage amount to treat the immune-mediated inflammatory disease, then the subject is treated with a therapeutic agent other than the biologic drug. The type, dose and inter-dose interval of biologic drug may vary depending on the percentage reduction in hazard remission. In some embodiments, the immune-mediated inflammatory disease is inflammatory bowel disease, such as Crohn's disease (CD) or ulcerative colitis (UC).

### Methods of Detection

Disclosed herein are methods for analyzing biologic material in a sample to detect a presence, an absence, or a quantity of one or more analytes or biomarkers (e.g., nucleic acid sequence, protein, carbohydrate) in the sample. In some embodiments, the sample is obtained from a subject. In some embodiments, the analyte that is detected is the biologic drug disclosed herein, or an antibody against the biologic drug. The biologic drug may be one or more drugs provided elsewhere herein. In some embodiments, the analyte that is detected a target protein. In some embodiments, the target protein is albumin, C-reactive protein (CRP), or a serological marker disclosed herein. In some embodiments, the serological marker comprises perinuclear antineutrophil cytoplasmic antibodies (pANCA), proteinase 3 (PR3), or antibodies against: α4β1, αE, α4, β7, β8, ITGAL, ICAM1, any dimers thereof, or any combinations thereof. In some embodiments the target protein comprises antibodies against the biologic drug disclosed herein. In some embodiments, methods of detection disclosed herein comprise detecting any combination of the above target proteins. A target protein or biologic drug may be detected by use of an antibody-based assay, where an antibody specific to the target protein is utilized. In some embodiments, antibody-based detection methods utilize an antibody that binds to any region of target protein. An exemplary method of analysis comprises performing an enzyme-linked immunosorbent assay (ELISA). The ELISA assay may be a sandwich ELISA or a direct ELISA. Another exemplary method of analysis comprises a single molecule array, e.g., Simoa. Other exemplary methods of detection include immunohistochemistry and lateral flow assay. Additional exemplary methods for detecting target protein include, but are not limited to, gel electrophoresis, capillary electrophoresis, high performance liquid chromatography (HPLC), thin layer chromatography (TLC), hyperdiffusion chromatography, and the like, or various immunological methods such as fluid or gel precipitation reactions, immunodiffusion (single or double), immunoelectrophoresis, radioimmunoassay (RIA), immunofluorescent assays, and Western blotting. In some embodiments, antibodies, or antibody fragments, are used in methods such as Western blots or immunofluorescence techniques to detect the expressed proteins. The antibody or protein can be immobilized on a solid support for Western blots and immunofluorescence techniques. Suitable solid phase supports or carriers include any support capable of binding an antigen or an antibody. Exemplary supports or carriers include glass, polystyrene, polypropylene, polyethylene, dextran, nylon, amylases, natural and modified celluloses, polyacrylamides, gabbros, and magnetite.

In some cases, a target protein may be detected by detecting binding between the target protein and a binding partner of the target protein. Exemplary methods of analysis of protein-protein binding comprise performing an assay in vivo or in vitro, or ex vivo. In some instances, the method of analysis comprises an assay such as a co-immunoprecipitation (co-IP), pull-down, crosslinking protein interaction analysis, labeled transfer protein interaction analysis, or Far-western blot analysis, FRET based assay, including, for example FRET-FLIM, a yeast two-hybrid assay, BiFC, or split luciferase assay.

Disclosed herein are methods of detecting a presence or a level of one or more serological markers in a sample obtained from a subject. In some embodiments, the one or more serological markers comprises anti-Saccharomyces cerevisiae antibody (ASCA), an anti-neutrophil cytoplasmic antibody (ANCA), antibody against E. coli outer membrane porin protein C (anti-OmpC), anti-chitin antibody, pANCA antibody, anti-I2 antibody, and anti-Cbir1 flagellin antibody. In some embodiments, the serological marker comprises pANCA. In some embodiments, the serological marker comprises proteinase 3 (PR3). In some embodiments, the serological marker comprises antibodies against: α4β1, αE, α4, β7, β8, ITGAL, ICAM1, any dimers thereof, or any combinations thereof. In some embodiments, the antibodies comprises immunoglobulin A (IgA), immunoglobulin G (IgG), immunoglobulin E (IgE), or immunoglobulin M (IgM), immunoglobulin D (IgD), or a combination thereof. Any suitable method for detecting a target protein or biomarker disclosed herein may be used to detect a presence, absence, or level of a serological marker. In some embodiments, the presence or the level of the one or more serological markers is detected using an enzyme-linked immunosorbent assay (ELISA), a single molecule array (Simoa), immunohistochemistry, internal transcribed spacer (ITS) sequencing, or any combination thereof. In some embodiments, the ELISA is a fixed leukocyte ELISA. In some embodiments, the ELISA is a fixed neutrophil ELISA. A fixed leukocyte or neutrophil ELISA may be useful for the detection of certain serological markers, such as those described in Saxon et al., A distinct subset of antineutrophil cytoplasmic antibodies is associated with inflammatory bowel disease, J. Allergy Clin. Immuno. 86:2; 202-210 (August 1990), which is hereby incorporated by reference in its entirety. In some embodiments, ELISA units (EU) are used to measure positivity of a presence or level of a serological marker (e.g., seropositivity), which reflects a percentage of a standard or reference value. In some embodiments, the standard comprises pooled sera obtained from well-characterized patient population (e.g., diagnosed with the same disease or condition the subject has, or is suspected of having) reported as being seropositive for the serological marker of interest. In some embodiments, the control or reference value comprises 10, 20, 30, 40, 50, 60, 70, 80, 90, or 100 EU. In some instances, a quartile sum scores are calculated using, for example, the methods reported in Landers C J, Cohavy O, Misra R. et al., Selected loss of tolerance evidenced by Crohn's disease-associated immune responses to auto- and microbial antigens. Gastroenterology (2002)123:689-699, which is hereby incorporated by reference in its entirety.

In some embodiments, the analyte is a nucleic acid sequence. In some embodiments, the nucleic acid sequence comprises one or more polymorphisms. In some embodiments, the sample is assayed to measure a presence, absence, or quantity the one or more polymorphisms. In some embodiments, the polymorphism comprises a copy number variant, a single nucleotide variation (SNV), or an indel (e.g., insertion/deletion). In some embodiments, the SNV detected is at rs2097432 or a SNV in linkage disequilibrium (LD) therewith. In some embodiments, the SNV in LD with rs2097432 is determined by a r² value of 0.80.

In some cases, the nucleic acid sequence comprises DNA. In some instances, the nucleic acid sequence comprises a denatured DNA molecule or fragment thereof. In some instances, the nucleic acid sequence comprises DNA selected from: genomic DNA, viral DNA, mitochondrial DNA, plasmid DNA, amplified DNA, circular DNA, circulating DNA, cell-free DNA, or exosomal DNA. In some instances, the DNA is single-stranded DNA (ssDNA), double-stranded DNA, denaturing double-stranded DNA, synthetic DNA, and combinations thereof. The circular DNA may be cleaved or fragmented. In some instances, the nucleic acid sequence comprises RNA. In some instances, the nucleic acid sequence comprises fragmented RNA. In some instances, the nucleic acid sequence comprises partially degraded RNA. In some instances, the nucleic acid sequence comprises a microRNA or portion thereof. In some instances, the nucleic acid sequence comprises an RNA molecule or a fragmented RNA molecule (RNA fragments) selected from: a microRNA (miRNA), a pre-miRNA, a pri-miRNA, a mRNA, a pre-mRNA, a viral RNA, a viroid RNA, a virusoid RNA, circular RNA (circRNA), a ribosomal RNA (rRNA), a transfer RNA (tRNA), a pre-tRNA, a long non-coding RNA (lncRNA), a small nuclear RNA (snRNA), a circulating RNA, a cell-free RNA, an exosomal RNA, a vector-expressed RNA, an RNA transcript, a synthetic RNA, and combinations thereof.

Nucleic acid-based detection techniques that may be useful for the methods herein include quantitative polymerase chain reaction (qPCR), gel electrophoresis, immunochemistry, in situ hybridization such as fluorescent in situ hybridization (FISH), cytochemistry, and next generation sequencing. In some embodiments, the methods involve TaqMan™ qPCR, which involves a nucleic acid amplification reaction with a specific primer pair, and hybridization of the amplified nucleic acids with a hydrolysable probe specific to a target nucleic acid.

In some instances, the methods involve hybridization and/or amplification assays that include, but are not limited to, Southern or Northern analyses, polymerase chain reaction analyses, and probe arrays. Non-limiting amplification reactions include, but are not limited to, qPCR, self-sustained sequence replication, transcriptional amplification system, Q-Beta Replicase, rolling circle replication, or any other nucleic acid amplification known in the art. As discussed, reference to qPCR herein includes use of TaqMan™ methods. An additional exemplary hybridization assay includes the use of nucleic acid probes conjugated or otherwise immobilized on a bead, multi-well plate, or other substrate, wherein the nucleic acid probes are configured to hybridize with a target nucleic acid sequence of a genotype provided herein. A non-limiting method is one employed in Anal Chem. 2013 Feb. 5; 85(3):1932-9.

In one embodiment, detecting the analyte is performed at the nucleic acid level by performing RNA-seq, a reverse transcriptase polymerase chain reaction (RT-PCR) or a hybridization assay with oligonucleotides that are substantially complementary to portions of cDNA molecules of the at least one biomarker gene under conditions suitable for RNA-seq, RT-PCR or hybridization and obtaining expression levels of the at least one biomarker gene.

In another embodiment, detecting the analyte is performed at the nucleic acid level by performing DNA sequencing as described herein, a polymerase chain reaction (PCR, e.g., real time PCR or quantitative PCR) and/or a hybridization assay with oligonucleotides that are substantially complementary to portions of amplified DNA molecules of the gene under conditions suitable for hybridization, thereby obtaining the genotype of the biomarker genes.

In some embodiments, detecting the analyte comprises sequencing genetic material from the subject. Sequencing can be performed with any appropriate sequencing technology, including but not limited to single-molecule real-time (SMRT) sequencing, Polony sequencing, sequencing by ligation, reversible terminator sequencing, proton detection sequencing, ion semiconductor sequencing, nanopore sequencing, electronic sequencing, pyrosequencing, Maxam-Gilbert sequencing, chain termination (e.g., Sanger) sequencing, +S sequencing, or sequencing by synthesis. Sequencing methods also include next-generation sequencing, e.g., modern sequencing technologies such as Illumina sequencing (e.g., Solexa), Roche 454 sequencing, Ion torrent sequencing, and SOLiD sequencing. In some cases, next-generation sequencing involves high-throughput sequencing methods. Additional sequencing methods available to one of skill in the art may also be employed.

Examples of molecules that are utilized as probes include, but are not limited to, RNA and DNA. In some embodiments, the term “probe” with regards to nucleic acids, refers to any molecule that is capable of selectively binding to a specifically intended target nucleic acid sequence. In some instances, probes are specifically designed to be labeled, for example, with a radioactive label, a fluorescent label, an enzyme, a chemiluminescent tag, a colorimetric tag, or other labels or tags that are known in the art. In some instances, the fluorescent label comprises a fluorophore. In some instances, the fluorophore is an aromatic or heteroaromatic compound. In some instances, the fluorophore is a pyrene, anthracene, naphthalene, acridine, stilbene, benzoxazole, indole, benzindole, oxazole, thiazole, benzothiazole, canine, carbocyanine, salicylate, anthranilate, xanthenes dye, coumarin. Exemplary xanthene dyes include, e.g., fluorescein and rhodamine dyes. Fluorescein and rhodamine dyes include, but are not limited to 6-carboxyfluorescein (FAM), 2′7′-dimethoxy-4′5′-dichloro-6-carboxyfluorescein (JOE), tetrachlorofluorescein (TET), 6-carboxyrhodamine (R6G), N,N,N; N′-tetramethyl-6-carboxyrhodamine (TAMRA), 6-carboxy-X-rhodamine (ROX). Suitable fluorescent probes also include the naphthylamine dyes that have an amino group in the alpha or beta position. For example, naphthylamino compounds include 1-dimethylaminonaphthyl-5-sulfonate, 1-anilino-8-naphthalene sulfonate and 2-p-toluidinyl-6-naphthalene sulfonate, 5-(2′-aminoethyl)aminonaphthalene-1-sulfonic acid (EDANS). Exemplary coumarins include, e.g., 3-phenyl-7-isocyanatocoumarin; acridines, such as 9-isothiocyanatoacridine and acridine orange; N-(p-(2-benzoxazolyl)phenyl) maleimide; cyanines, such as, e.g., indodicarbocyanine 3 (Cy3), indodicarbocyanine 5 (Cy5), indodicarbocyanine 5.5 (Cy5.5), 3-(-carboxy-pentyl)-3′-ethyl-5,5′-dimethyloxacarbocyanine (CyA); 1H, 5H, 11H, 15H-Xantheno[2,3, 4-ij: 5,6, 7-i′j′]diquinolizin-18-ium, 9-[2 (or 4)-[[[6-[2,5-dioxo-1-pyrrolidinyl)oxy]-6-oxohexyl]amino]sulfonyl]-4 (or 2)-sulfophenyl]-2,3, 6,7, 12,13, 16,17-octahydro-inner salt (TR or Texas Red); or BODIPY™ dyes. In some cases, the probe comprises FAM as the dye label.

In some instances, primers and/or probes described herein for detecting a target nucleic acid are used in an amplification reaction. In some instances, the amplification reaction is qPCR. An exemplary qPCR is a method employing a TaqMan™ assay. PCR primers and probes can be designed with tools known and used in the art. For example, forward and reverse primers for regions containing SNPs can be designed by uploading the flanking sequences into the Thermofisher OligoPerfect Primer Designer tool. The primer set with longest amplicon can be selected for the forward and reverse primers. Flanking sequences of SNPs can be obtained from the NCBI dbSNP database. Probes can be designed with the Thermofisher SNP genotype tool. The resulting probe design from the SNP genotype tool can be then truncated to 10-20 nucleotide flanks for the final design.

In some instances, qPCR comprises using an intercalating dye. Examples of intercalating dyes include SYBR green I, SYBR green II, SYBR gold, ethidium bromide, methylene blue, Pyronin Y, DAPI, acridine orange, Blue View or phycoerythrin. In some instances, the intercalating dye is SYBR.

In some instances, a number of amplification cycles for detecting a target nucleic acid in an amplification assay is about 5 to about 30 cycles. In some instances, the number of amplification cycles for detecting a target nucleic acid is at least about 5 cycles. In some instances, the number of amplification cycles for detecting a target nucleic acid is at most about 30 cycles. In some instances, the number of amplification cycles for detecting a target nucleic acid is about 5 to about 10, about 5 to about 15, about 5 to about 20, about 5 to about 25, about 5 to about 30, about 10 to about 15, about 10 to about 20, about 10 to about 25, about 10 to about 30, about 15 to about 20, about 15 to about 25, about 15 to about 30, about 20 to about 25, about 20 to about 30, or about 25 to about 30 cycles.

In some embodiments, methods provided herein comprise extracting nucleic acids from the sample using any technique that does not interfere with subsequent analysis. In certain embodiments, this technique uses alcohol precipitation using ethanol, methanol, or isopropyl alcohol. In certain embodiments, this technique uses phenol, chloroform, or any combination thereof. In certain embodiments, this technique uses cesium chloride. In certain embodiments, this technique uses sodium, potassium or ammonium acetate or any other salt commonly used to precipitate DNA. In certain embodiments, this technique utilizes a column or resin based nucleic acid purification scheme such as those commonly sold commercially, one non-limiting example would be the GenElute Bacterial Genomic DNA Kit available from Sigma Aldrich. In certain embodiments, after extraction the nucleic acid is stored in water, Tris buffer, or Tris-EDTA buffer before subsequent analysis. In an exemplary embodiment, the nucleic acid material is extracted in water. In some cases, extraction does not comprise nucleic acid purification. In an exemplary embodiment, the nucleic acid material is extracted in water. In some cases, extraction does not comprise nucleic acid purification. In certain embodiments, RNA may be extracted from cells using RNA extraction techniques including, for example, using acid phenol/guanidine isothiocyanate extraction (RNAzol B; Biogenesis), RNeasy RNA preparation kits (Qiagen) or PAXgene (PreAnalytix, Switzerland).

In some embodiment, methods of detection comprise performing a qPCR assay, in which the nucleic acid sample is combined with primers and probes specific for a target nucleic acid that may or may not be present in the sample, and a DNA polymerase. An amplification reaction is performed with a thermal cycler that heats and cools the sample for nucleic acid amplification, and illuminates the sample at a specific wavelength to excite a fluorophore on the probe and detect the emitted fluorescence. For TaqMan™ methods, the probe may be a hydrolysable probe comprising a fluorophore and quencher that is hydrolyzed by DNA polymerase when hybridized to a target nucleic acid. In some cases, the presence of a target nucleic acid is determined when the number of amplification cycles to reach a threshold value is less than 30, 29, 28, 27, 26, 25, 24, 23, 22, 21, or 20 cycles.

In some embodiments, the sample is obtained from the subject or patient indirectly or directly. In some embodiments, the sample may be obtained by the subject. In other embodiments, the sample may be obtained by a healthcare professional, such as a nurse or physician. The sample may be derived from virtually any biological fluid or tissue containing genetic information, such as blood.

### Methods of Predicting Time to Disease Remission

Disclosed herein, in some embodiments, are methods for predicting, using one or more statistical models disclosed herein, whether an immune-mediated inflammatory disease in a subject will achieve disease remission with a particular dose and/or inter-dose interval of an biologic drug. In some embodiments, if the predicted dose or inter-dose interval is predicted to exceed an approved dosage amount for the immune-mediated inflammatory disease, then methods further comprise predicting that the subject will not achieve disease remission with the biologic drug. In some embodiments, the biologic drug is or comprises an anti-TNF therapy.

In some embodiments, the methods comprise calculating an estimated clearance rate at which a biologic drug (e.g., anti-TNF therapy) is cleared from the subject using a first statistical model based, at least in part, on a level of albumin measured in a biological sample obtained from the subject, and a weight of the subject; and comparing the clearance rate calculated to a predetermined threshold, to produce poor prognosis factor 1 (PPF1) score on a scale of 0 to 1, wherein 0 is indicative of a low rate of clearance below the predetermined threshold, and 1 is indicative of a high rate of clearance above the predetermined threshold. In some embodiments, the first statistical model comprises a Naïve Bayes classifier algorithm, a non-linear mixed effects model (NLME), or a Metropolis Hastings algorithm, or any combination thereof. In some embodiments, the first statistical model is trained using data received from a reference population that were or currently are being treated with the biologic drug for treatment of the immune-mediated inflammatory disease of the subject. Subject specific parameters may be generated and disease remission for the subject may be simulated based on the subject specific parameters and the data from the reference population. In some embodiments, the subject specific parameters are provided in Table 12. In some embodiments, albumin levels measured in a biological sample obtained from the subject may be obtained using homogenous mobility shift assays or solid phase assays. In some embodiments, a biological sample is obtained from the subject indirectly or directly. In some embodiments, the sample may be obtained by the subject. In some embodiments, the sample may be obtained by a healthcare professional, such as a nurse or physician. The sample may be derived from virtually any biological fluid or tissue containing genetic information, such as blood. In some embodiments, the blood is capillary blood (such as, for example, blood obtained from a finger prick). In some embodiments, the clearance is measured at baseline, which refers to a measurement of albumin in the biological sample collected at baseline before the subject starts the biologic drug. In some embodiments, the clearance of albumin and the subject's weight are used as covariates in estimating the clearance of the therapeutic agent. In some embodiments, higher clearance may be indicative of a higher likelihood for a subject to have a reduction in the hazard function (remission). In some embodiments, methods comprise calculating, by the pharmacokinetic system, a PPF1 score, which is indicative of a likelihood of clearance of the therapeutic agent from the subject. In some embodiments, a score of 1 may be calculated if the subject has a high likelihood of a high clearance of the therapeutic agent (and thus, a high reduction in the hazard function (remission)). In some embodiments, a score of 0 may be calculated if the subject has a low likelihood of a high clearance of the therapeutic agent (and thus, a low reduction in the hazard function (remission)). In some embodiments, a rate of which a biologic drug is estimated to be cleared from the subject based on the first quantity and the second quantity measured, and comparing the rate to a predetermined threshold. In some embodiments, comparing the rate to a predetermined threshold may produce an estimated clearance score on a scale of 0 to 1. In some embodiments, the predetermined threshold is about 0.1 to 0.5 liters per day (L/day), about 0.1 to 0.4 L/day, about 0.1 to 0.3 L/day, about 0.1 to 0.2 L/day, about 0.2 to 0.5 L/day, about 0.2 to 0.4 L/day, about 0.2 to 0.3 L/day, about 0.3 to 0.5 L/day, about 0.3 to 0.4 L/day, or about 0.4 to 0.5 L/day. In some embodiments, the predetermined threshold is about 0.1 L/day, about 0.2 L/day, about 0.3 L/day, about 0.4 L/day, or about 0.5 L/day. In some embodiments, the predetermined threshold is about 0.2 L/day, 0.21 L/day, 0.22 L/day, 0.23 L/day, 0.24 L/day, 0.25 L/day, 0.26 L/day, 0.27 L/day, 0.28 L/day, or 0.29 L/day. In some embodiments, the predetermined threshold is about 0.294 L/day. In some embodiments, the predetermined threshold is at least about 0.1 L/day, about 0.2 L/day, about 0.3 L/day, about 0.4 L/day, or about 0.5 L/day. In some embodiments, the predetermined threshold is above about 0.1 L/day, about 0.2 L/day, about 0.3 L/day, about 0.4 L/day, or about 0.5 L/day.

In some embodiments, the methods comprise calculating a poor prognosis factor 2 (PPF2) score on a scale of 0 to 2 with a second statistical model, based at least in part, on a presence of a genotype at a single nucleotide variant (SNV) comprising rs2097432 or a SNV in linkage disequilibrium therewith detected in the biological sample obtained from the subject, wherein 0 is indicative of the genotype that is homozygous non-risk, 1 is indicative of the genotype that is heterozygous risk at rs2097432, and 2 comprises a presence of two genetic loci of said genotype. In some embodiments, the second statistical algorithm comprises a univariate logistic regression algorithm. In some embodiments, the genotype comprises one or more two nucleotides at single nucleotide variant rs2097432, located chromosome 6, nucleotide position 32622994 (GRCh38) or chromosome 6, nucleotide position 32590771 (GRCh37). In some embodiments, the nucleotide at nucleotide position 32622994 (GRCh38) or 32590771 (GRCh37) is a guanine (“G”). In some embodiments, the genotype comprises an SNV in linkage disequilibrium with rs2097432. Linkage disequilibrium may be evaluated based on a D′ value that is at least 0.20, or an r2 value comprising at least 0.70, but preferably 0.85. In some embodiments, the genotype is homozygous for the risk allele, heterozygous for the risk allele, or homozygous for the non-risk allele. In some embodiments, the genotype for the risk allele may be homozygous (e.g., GG). In some embodiments, the genotype for the risk allele may be heterozygous (e.g., AG). In some embodiments, the genetic system receives genotype data for the subject from a genotyping device, such as a sequencer, a genotype array, or the like. In some embodiments, the “G” at nucleotide position 32622994 (GRCh38) or 32590771 (GRCh37) is associated an increased risk of developing antibodies to Infliximab or antibodies to adalimumab. In some embodiments, the “G” at nucleotide position 32622994 (GRCh38) or 32590771 (GRCh37) is also associated with increased risk of loss of response to Infliximab.

In some embodiments, the methods comprise calculating a poor prognosis factor 3 (PPF3) score on a scale of 0 to 2 with a third statistic model, wherein 0 is indicative of a standard response to the biologic drug, 1 is indicative e of a low response to the biologic drug, and 2 is indicative of very low response to the biologic drug. In some embodiments, a score of 2 is achieved with PR3 levels above the 99^{th} percentile of a reference population of subjects without the immune-mediated inflammatory disease (e.g., IBD), or presence of pANCA, or a combination thereof. In some embodiments, the score of 2 further comprises CRP levels above 3 mg/L. In some embodiments, the score of 0 is achieved with PR3 levels in the 99^{th} percentile of a reference population of subjects without the immune-mediated inflammatory disease, or absence of pANCA, or a combination thereof. In some embodiments, the score of 0 further comprises CRP levels below 3 mg/L. In some embodiments, the third statistic model is a multivariate linear or logistic algorithm.

In some embodiments, the methods comprise calculating a poor prognosis factor 4 (PPF4) score on a scale of 0 to 1 with a fourth statistical model, based at least in part on patient wellbeing received from the subject, wherein a PPF4 score of 0 is indicative of an improvement of at least one symptom of the immune-mediated inflammatory disease, and a PPF4 score of 1 is indicative of no improvement in the at least one symptom, and wherein the combined PPF score calculated further comprises the PPF4 score. In some embodiments, the patient wellbeing data may be received using a mobile application displayed to the subject via a GUI, as exemplary illustrated in FIG. 8. In some embodiments, the mobile application may include patient questions. Non-limiting examples of patient questions may include: How many liquid or very soft stools did you have today?; How was your abdominal pain today?; How was your stool frequency today?; and How was your rectal bleeding today? In some embodiments, patient wellbeing data may be received an analysis technique. In some embodiments, the analysis technique may comprise flow cytometry. In some embodiments, flow cytometry may comprise measuring a physical or a chemical characteristics associated with a biological sample obtained from a patient. In some embodiments, the fourth statistic model is a multivariate linear or logistic algorithm.

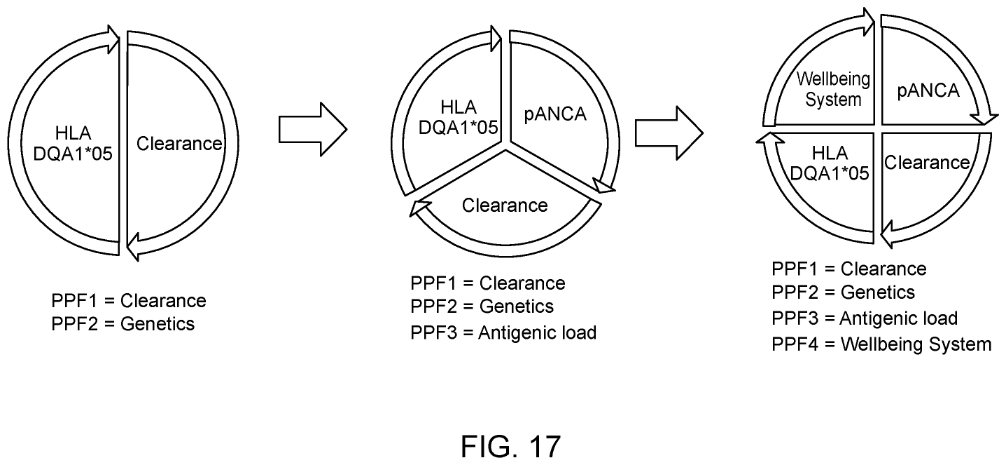

In some embodiments, the methods further comprising calculating a combined PPF score comprising two or more of the PPF1 score, the PPF2 score, the PPF3 score, and the PPF4 score, to predict whether the immune-mediated inflammatory disease in the subject will positively respond to the treatment with the biologic drug (e.g., whether disease remission is estimated by the model within the approve dose and inter-dose interval ranges for the biologic drug). In some embodiments, methods comprise aggregating the PPF1 score and PPF 2 score to produce a combined PPF score as illustrated in FIG. 17. The aggregated scores may be correlated to the time to remission of a disease. In some embodiments, the correlation may be established using an algorithm for optimization and/or parameterizations with Hazard Function on Time to event during proactive monitoring of a biologic drug disclosed herein (e.g., Infliximab). In some cases, parametric estimation may comprise optimizing parameters in a numerical model. In some instances, parametric estimation may comprise minimizing a cost function related to a dataset. In some embodiments, the disease may be inflammatory bowel disease. In some embodiments, a subject who have a combined PPF score (e.g., PPF1+PPF2) of a certain value may receive a therapeutic agent comprising a biologic drug. In some embodiments, a subject who have a combined PPF score (e.g., PPF1+PPF2) of a certain value may receive a therapeutic agent other than the biologic drug. In some embodiments, a the biologic drug comprises an inhibitor of interleukin 12 (IL-12) or interleukin 23 (IL-23). In some embodiments, the inhibitor of IL-12 or IL-23 is or comprises Ustekinumab. In some embodiments, the therapy other than the biologic drug is or comprises an agent that targets α4β7 integrin. In some embodiments, the agent that targets α4β7 integrin is or comprises Vedolizumab. In some embodiments, the therapeutic agent other than an anti-TNF therapy comprises a small molecule JAK inhibitor. In some embodiments, the small molecule JAK inhibitor comprises Baricitinib, Tofacitinib, or Upadacitinib, or any combination thereof. In some embodiments, the therapeutic agent other than a biologic drug comprises a sphingosine 1-phosphate (S1P) receptor modulator. In some embodiments, the S1P receptor modulator comprises Fingolimod, Siponimod, Ozanimod, or Ponesimod, or any combination thereof. In some embodiments, the biologic drug comprises Infliximab, Adalimumab, Eetanercept, Golimumab, or Certolizumab, or any combination thereof.

In some embodiments, methods further comprise aggregating the PPF 3 scores (antigenic load system) and/or PPF 4 scores (patient wellbeing system) with the PPF 1 and PPF 2 scores to produce the combined PPF score, as further illustrated in FIG. 17. In some embodiments, as shown in FIG. 17, the PPF3 score may comprise detecting an antineutrophil cytoplasmic antibodies (e.g., ANCA, pANCA). In some embodiments, PPF4 may be determined using analyzing patient wellbeing data. As such, the model may further involve consideration of risk immune status of the subject, as well as one or more symptoms related to inflammation experienced by the subject. In some embodiments, the inflammation may be due to inflammatory bowel disease. In some embodiments, the model may comprise a parametric estimation algorithm. The parametric estimation may be used to determine the parameters associated with the respective scored described herein. In some embodiments, the parameters are provided in Table 12. In some embodiments, the PPF 1, PPF 2, PPF 3, and PPF 4 scores are aggregated to produce the combined PPF score for the subject.

In some embodiments, the PPF or combined PPF score represents a time to remission (e.g., time that the immune-mediated inflammatory disease will be in remission in the subject. In some embodiments, the methods further comprise converting the time to remission to a percentage reduction in hazard remission. In some embodiments, the model comprises one or more algorithms. In some embodiments, an algorithm is initialized with parameters derived from data from a clinical population. The model may derive clinical population data for an immune-mediated inflammatory disease, such as inflammatory bowel disease. In some embodiments, the clinical population data may include data taken from a typical population of subjects having the immune-mediated inflammatory disease. In some embodiments, the clinical population data may include subject population data including serological markers, genetic markers, general subject information (e.g., age, weight, gender, etc.), and drug concentration levels in subjects being administered drugs in differing dosing regimens.

Methods further comprise, in some embodiments, administering or selecting the subject for treatment with, a specific dosage of the therapeutic agent based on the combined PPF score calculated by the patient-centric precision model disclosed herein. In some embodiments, if the model estimates that the subject will achieve disease remission with the biologic drug that is over the maximum dose for that drug, then the subject is treated with another therapeutic agent disclosed herein. In some embodiments, the another therapeutic agent may be a small molecule inhibitor. In some embodiments, the small molecule inhibitor is specific to a Janus Kinase (JAK). In some embodiments, the small molecule inhibitor specific to JAK comprises baricitinib, tofacitinib, or upadacitinib, or any combination thereof. In some embodiments, the small molecule is specific to a sphingosine 1-phosphate (S1P) modulator or an S1P receptor modulator. In some embodiments, the small molecule specific to an S1P receptor modulator comprises fingolimod, siponimod, ozanimod, or ponesimod, or any combination thereof.

In some embodiments, the subject is administered 5 mg/Kg of the therapeutic agent when the combined PPF score is 0. In some embodiments, the subject is administered 7.5 mg/Kg of the therapeutic agent when the combined PPF score is 1. In some embodiments, the subject is administered 10 mg/Kg of the therapeutic agent when the combined PPF score is 2. In some embodiments, the subject is administered a dose of a biologic drug provided in Table 15. In some embodiments, the subject is administered a different therapeutic agent when the combined PPF score is 3. In some embodiments, the therapeutic agent is an biologic drug. In some embodiments, the anti-TNF therapy comprises Infliximab, Adalimumab, Eetanercept, Golimumab, or Certolizumab. In some embodiments, the different therapeutic is or comprises an inhibitor of interleukin 12 (IL-12) or interleukin 23 (IL-23). In some embodiments, the inhibitor of IL-12 or IL-23 is or comprises Ustekinumab. In some embodiments, the different therapeutic is or comprises an agent that targets α4β7 integrin. In some embodiments, the agent that targets α4β7 integrin is or comprises Vedolizumab. In some embodiments, the different therapeutic is or comprises a small molecule JAK inhibitor. In some embodiments, the small molecule JAK inhibitor comprises Baricitinib, Tofacitinib, or Upadacitinib, or any combination thereof. In some embodiments, the different therapeutic is or comprises a sphingosine 1-phosphate (S1P) receptor modulator. In some embodiments, the S1P receptor modulator comprises Fingolimod, Siponimod, Ozanimod, or Ponesimod, or any combination thereof.

In some embodiments, the poor prognosis factors are measured for subjects who have not received an biologic drug. In some cases, subjects who have not received a biologic drug may be referred to as those whose performances are measured in a proactive setting. In some embodiments, the poor prognosis factors are measured for subjects who have received a biologic drug. In some cases, subjects who have received a biologic drug are referred to as those whose performances are measured in a reactive setting.

In some embodiments, the poor prognosis factors may be combined with proactive maintenance of a biologic drug (e.g., Infliximab) to predict a time to remission in a subject. In some cases, a proactive maintenance of the biologic drug levels may be between about 10 to 20 mg/L, about 10 to 30 mg/L, about 10 to 40 mg/L, about 10 to 50 mg/L, about 5 to 20 mg/L, about 5 to 30 mg/L, or about 5 to 40 mg/L. In some cases, the proactive maintenance of the biologic drug may be between about 5 mg/L, about 10 mg/L, about 15 mg/L, about 20 mg/L, about 25 mg/L, about 30 mg/L, about 35 mg/L, or about 40 mg/L. In some cases, a proactive maintenance of Infliximab (IFX) levels may be between about 10 to 20 mg/L, about 10 to 30 mg/L, about 10 to 40 mg/L, about 10 to 50 mg/L, about 5 to 20 mg/L, about 5 to 30 mg/L, or about 5 to 40 mg/L. In some embodiments, the biologic drug comprises an anti-TNF therapy (e.g., infliximab adalimumab). In some cases, an anti-TNF therapy level may be between about 5 mg/L, about 10 mg/L, about 15 mg/L, about 20 mg/L, about 25 mg/L, about 30 mg/L, about 35 mg/L, or about 40 mg/L.

In some embodiments, methods comprise estimating that the subject will not achieve disease remission with the biologic drug (e.g., anti-TNF therapy) with a confidence comprising about 50% to 95%. In some embodiments, the confidence comprises about 50% to 95%, 55% to 90%, 60% to 85%, 65% to 80%, or 70% to 75%. In some embodiments, the confidence comprises about 50%, 55%, 60%, 65%, 70%, 75%, 80%, 85% 90%, 91%, 92%, 93%, 94%, or 95%. In some embodiments, the confidence is at least about 50%, 55%, 60%, 65%, 70%, 75%, 80%, 85%, 90%, 91%, 92%, 93%, 94%, or 95%. In some embodiments, the confidence is at least about 90%.

In some embodiments, methods comprise estimating that the dose of the biologic drug at the inter-dose interval of the biologic drug (e.g., anti-TNF therapy) to achieve disease remission with a specificity comprising about 50% to 95%. In some embodiments, the specificity comprises about 50% to 95%, 55% to 90%, 60% to 85%, 65% to 80%, or 70% to 75%. In some embodiments, the specificity comprises about 50%, 55%, 60%, 65%, 70%, 75%, 80%, 85%, 90%, 91%, 92%, 93%, 94%, or 95%. In some embodiments, the specificity is at least about 50%, 55%, 60%, 65%, 70%, 75%, 80%, 85%, 90%, 91%, 92%, 93%, 94%, or 95%. In some embodiments, the specificity is at least about 90%.

In some embodiments, methods comprise estimating that the subject will not achieve disease remission with the biologic drug (e.g., anti-TNF therapy) with a specificity comprising about 50% to 95%. In some embodiments, the specificity comprises about 50% to 95%, 55% to 90%, 60% to 85%, 65% to 80%, or 70% to 75%. In some embodiments, the specificity comprises about 50%, 55%, 60%, 65%, 70%, 75%, 80%, 85%, 90%, 91%, 92%, 93%, 94%, or 95%. In some embodiments, the specificity is at least about 50%, 55%, 60%, 65%, 70%, 75%, 80%, 85%, 90%, 91%, 92%, 93%, 94%, or 95%. In some embodiments, the specificity is at least about 90%.

In some embodiments, methods comprise estimating the dose of the biologic drug at the inter-dose interval of the biologic drug (e.g., anti-TNF therapy) to achieve disease remission with a positive predictive value comprising about 50% to 955%. In some embodiments, the positive predictive value comprises about 50% to 95%, 55% to 90%, 60% to 85%, 65% to 80%, or 70% to 75%. In some embodiments, the positive predictive value comprises about 50%, 55%, 60%, 65%, 70%, 75%, 80%, 85%, 90%, 91%, 92%, 93%, 94%, or 95%. In some embodiments, the positive predictive value is at least about 50%, 55%, 60%, 65%, 70%, 75%, 80%, 85%, 90%, 91%, 92%, 93%, 94%, or 95%. In some embodiments, the positive predictive value is at least about 90%.

In some embodiments, methods comprise estimating that the subject will not achieve disease remission with the biologic drug (e.g., anti-TNF therapy) with a positive predictive value comprising about 50% to 95%. In some embodiments, the positive predictive value comprises about 50% to 95%, 55% to 90%, 60% to 85%, 65% to 80%, or 70% to 75%. In some embodiments, the positive predictive value comprises about 50%, 55%, 60%, 65%, 70%, 75%, 80%, 85%, 90%, 91%, 92%, 93%, 94%, or 95%. In some embodiments, the positive predictive value is at least about 50%, 55%, 60%, 65%, 70%, 75%, 80%, 85%, 90%, 91%, 92%, 93%, 94%, or 95%. In some embodiments, the positive predictive value is at least about 90%.

In some embodiments, methods comprise estimating the dose of the biologic drug at the inter-dose interval of the biologic drug (e.g., anti-TNF therapy) to achieve disease remission with a negative predictive value comprising about 50% to 95%. In some embodiments, the negative predictive value comprises about 50% to 95%, 55% to 90%, 60% to 85%, 65% to 80%, or 70% to 75%. In some embodiments, the negative predictive value comprises about 50%, 55%, 60%, 65%, 70%, 75%, 80%, 85%, 90%, 91%, 92%, 93%, 94%, or 95%. In some embodiments, the negative predictive value is at least about 50%, 55%, 60%, 65%, 70%, 75%, 80%, 85%, 90%, 91%, 92%, 93%, 94%, or 95%. In some embodiments, the negative predictive value is at least about 90%.

In some embodiments, methods comprise estimating that the subject will not achieve disease remission with the biologic drug (e.g., anti-TNF therapy) with a negative predictive value comprising about 50% to 95%. In some embodiments, the negative predictive value comprises about 50% to 95%, 55% to 90%, 60% to 85%, 65% to 80%, or 70% to 75%. In some embodiments, the negative predictive value comprises about 50%, 55%, 60%, 65%, 70%, 75%, 80%, 85%, 90%, 91%, 92%, 93%, 94%, or 95%. In some embodiments, the negative predictive value is at least about 50%, 55%, 60%, 65%, 70%, 75%, 80%, 85%, 90%, 91%, 92%, 93%, 94%, or 95%. In some embodiments, the negative predictive value is at least about 90%.

In some embodiments, methods comprise estimating the dose of the biologic drug at the inter-dose interval of the biologic drug (e.g., anti-TNF therapy) to achieve disease remission with an area under the curve (AUC) comprising about 0.50 to 0.95. In some embodiments, the area under the curve (AUC) comprises about 0.50 to 0.095, 0.55 to 0.90, 0.60 to 0.85, 0.65 to 0.80, or 0.70 to 0.75. In some embodiments, the area under the curve (AUC) comprises about 0.50, 0.55, 0.60, 0.65, 0.70, 0.75, 0.80, 0.85, 0.90, 0.91, 0.92, 0.93, 0.94, or 0.95. In some embodiments, the area under the curve (AUC) is at least about 0.50, 055, 0.60, 0.65, 0.70, 0.75, 0.80, 0.85, 0.90, 0.91, 0.92, 0.93, 0.94, or 0.95. In some embodiments, the area under the curve (AUC) is at least about 0.90.

In some embodiments, methods comprise estimating that the subject will not achieve disease remission with the biologic drug (e.g., anti-TNF therapy) with an area under the curve (AUC) comprising about 0.50 to 0.95. In some embodiments, the area under the curve (AUC) comprises about 0.50 to 0.095, 0.55 to 0.90, 0.60 to 0.85, 0.65 to 0.80, or 0.70 to 0.75. In some embodiments, the area under the curve (AUC) comprises about 0.50, 0.55, 0.60, 0.65, 0.70, 0.75, 0.80, 0.85, 0.90, 0.91, 0.92, 0.93, 0.94, or 0.95. In some embodiments, the area under the curve (AUC) is at least about 0.50, 055, 0.60, 0.65, 0.70, 0.75, 0.80, 0.85, 0.90, 0.91, 0.92, 0.93, 0.94, or 0.95. In some embodiments, the area under the curve (AUC) is at least about 0.90.

### Methods of Treatment and Treatment Optimization

Disclosed herein are methods of treating, or optimizing a treatment of, an immune-mediated inflammatory disease or condition disclosed herein, such as inflammatory bowel disease, in a subject. In some embodiments, the type and dose of therapeutic agent used to treat the immune-mediated inflammatory disease is based, at least in part, on the combined PPF calculated using the patient-centric precision model disclosed herein. In some embodiments, methods comprise administering a dose of a therapeutic agent to the subject, wherein the dose is determined using the patient-centric precision dosing model disclosed herein. Also provided herein are methods for optimizing a therapeutic regimen for the subject, such as therapeutic regiment including administration of a biological therapy. The biological therapy regiment may comprise treating a patient with a biologic drug or a small molecule. The subject may be a patient diagnosed with disease. In some embodiments, the disease comprises an immune-mediated inflammatory disease. In some embodiments, the disease comprises a cancer. In some embodiments, the systems and methods may involve inputting patient data into a model to forecast a drug concentration level in a patient and establish a dosing regimen for maintaining disease remission in the patient. The pre-specified threshold may be a target concentration level for effective treatment of the disease, such as an immune-mediated inflammatory disease, in the patient.

### Immune-Mediated Inflammatory Diseases

In some embodiments, the disease disclosed herein comprises an immune-mediated inflammatory disease. An immune-mediated disease may comprise, by way of non-limiting example, IBD, rheumatoid arthritis (RA), cytokine release syndrome, multiple sclerosis (MS), ankylosing spondylitis (AS), lupus, plaque psoriasis, atopic dermatitis, gout, migraine, or cancer. In some cases, the IBD comprises Crohn's disease (CD). In some cases, the IBD comprises ulcerative colitis (UC). In some embodiments, the disease comprises cancer. In some embodiments, the cancer comprises bladder cancer, breast cancer, cervical cancer, colorectal cancer, gynecologic cancer, kidney cancer, head and/or neck cancer, leukemia, liver cancer, lung cancer, lymphoma, mesothelioma, myeloma, ovarian cancer, prostate cancer, skin cancer, thyroid cancer, uterine cancer, vaginal or vulvar cancer. In some embodiments, lymphoma comprises Hodgkin lymphoma or non-Hodgkin lymphoma. In some embodiments, leukemia comprises acute lymphoblastic leukemia (ALL), acute myeloid leukemia (AML), chronic lymphocytic leukemia (CLL), chronic myelogenous leukemia (CML).

### Therapeutic Agent

Disclosed herein are therapeutic agents useful for treating a disease or a condition disclosed herein. In some embodiments, the therapeutic agent is a biologic drug. In some embodiments, the biologic drug comprises an antibody or an antigen-binding fragment thereof. In some embodiments, the antibody comprises a monoclonal antibody. In some embodiments, the monoclonal antibody is an antagonist against tumor necrosis factor (TNF), such as an anti-TNF antibody. In some embodiments, the biologic drug may be Infliximab (IFX), Adalimumab (ADA), Vedolizumab CDZ), or Ustekinumab (UST). In some embodiments, the biologic drug may be any of. Abatacept; Adalimumab (ADA); Alemtuzumab; Anakinra; Anti-tumor necrosis factor ligand 1 (TL1A); Apremilast; Azathioprine; Baricitinib; Belimumab; Benralizumab; Bimekizumab; Brodalumab; Canakinumab; Certolizumab pegol; Cyclosporine; Dupilumab; erenumab-aooe; Estrasimod; Etanercept; Etanercept; Etrolizumab; Filgotinib; fremanezumab-vfrm; Galcanezumab-gnlm; eptinezumab-jjmr, Golimumab; Golimumab Aria; Guselkumab; Hydroxychloroquine; Infliximab (IFX); Interferon Beta 1a; Interferon Beta 1b; Ixekizumab; Lebrikizumab; Leflunomide; Mepolizumab; Methotrexate; Mirikizumab; Mycophenolate; Natalizumab; Ocrelizumab; Ofatumumab; Ozanimod; peginterferon beta-1a; Pegloticase; reslizumab; Risankizumab-rzaa; Rituximab; Sarilumab; Secukinumab; Sulfasalazine; Tezepelumab; Tildrakizumab; Tocilizumab (TCZ); Tofacitinib; tralokinumab; Ublituximab; Upadacitinib; Ustekinumab; Vedolizumab or Semaglutide. In some embodiments, the small molecule comprises a small molecule inhibitor. In some embodiments, the small molecule inhibitor is specific to a Janus Kinase (JAK). In some embodiments, the small molecule inhibitor specific to JAK comprises baricitinib, tofacitinib, or upadacitinib, or any combination thereof. In some embodiments, the small molecule is specific to a sphingosine 1-phosphate (S1P) modulator or an S1P receptor modulator. In some embodiments, the small molecule specific to an S1P receptor modulator comprises fingolimod, siponimod, ozanimod, or ponesimod, or any combination thereof.

In some embodiments, the biologic drug may be Ado-trastuzumab emtansine (Kadcyla), Afatinib (Gilotrif), Aldesleukin (Proleukin), Alectinib (Alecensa), Alemtuzumab (Campath), Atezolizumab (Tecentriq), Avelumab (Bavencio), Axitinib (Inlyta), Belimumab (Benlysta), Belinostat (Beleodaq), Bevacizumab (Avastin), Blinatumomab (Blincyto), Bortezomib (Velcade), Bosutinib (Bosulif), Brentuximab vedotin (Adcetris), Brigatinib (Alunbrig), Cabozantinib (Cabometyx [tablet], Cometriq [capsule]), Canakinumab (Ilaris), Carfilzomib (Kyprolis), Ceritinib (Zykadia), Cetuximab (Erbitux), Cobimetinib (Cotellic), Crizotinib (Xalkori), Dabrafenib (Tafinlar), Daratumumab (Darzalex), Dasatinib (Sprycel), Denosumab (Xgeva), Dinutuximab (Unituxin), Durvalumab (Imfinzi), Elotuzumab (Empliciti), Enasidenib (Idhifa), Erlotinib (Tarceva), Everolimus (Afinitor), Gefitinib (Iressa), Ibritumomab tiuxetan (Zevalin), Ibrutinib (Imbruvica), Idelalisib (Zydelig), Imatinib (Gleevec), Ipilimumab (Yervoy), Ixazomib (Ninlaro), Lapatinib (Tykerb), Lenvatinib (Lenvima), Midostaurin (Rydapt), Necitumumab (Portrazza), Neratinib (Nerlynx), Nilotinib (Tasigna), Niraparib (Zejula), Nivolumab (Opdivo), Obinutuzumab (Gazyva), Ofatumumab (Arzerra, HuMax-CD20), Olaparib (Lynparza), Olaratumab (Lartruvo), Osimertinib (Tagrisso), Palbociclib (Ibrance), Panitumumab (Vectibix), Panobinostat (Farydak), Pazopanib (Votrient), Pembrolizumab (Keytruda), Pertuzumab (Perjeta), Ponatinib (Iclusig), Ramucirumab (Cyramza), Regorafenib (Stivarga), Ribociclib (Kisqali), Rituximab (Rituxan, Mabthera), Rituximab/hyaluronidase human (Rituxan Hycela), Romidepsin (Istodax), Rucaparib (Rubraca), Ruxolitinib (Jakafi), Siltuximab (Sylvant), Sipuleucel-T (Provenge), Sonidegib (Odomzo), Sorafenib (Nexavar), Temsirolimus (Torisel), Tocilizumab (Actemra), Tofacitinib (Xeljanz), Tositumomab (Bexxar), Trametinib (Mekinist), Trastuzumab (Herceptin), Vandetanib (Caprelsa), Vemurafenib (Zelboraf), Venetoclax (Venclexta), Vismodegib (Erivedge), Vorinostat (Zolinza), or Ziv-aflibercept (Zaltrap). In some embodiments, the patient is taking one of the above-mentioned biological therapies. In other embodiments, the patient may be taking more than one of the above-mentioned biological therapies.

In some embodiments, the of therapeutic agent comprises a biological or targeted therapy. In some embodiments, the biological therapy may be a monoclonal antibody. In some embodiments, the biological therapy may be Infliximab, Adalimumab, Vedolizumab, or Ustekinumab. In some embodiments, the targeted biological or small molecule therapy may be any of: Abatacept; Adalimumab; Adalimumab; Alemtuzumab; Anakinra; Anti-TL1a; Apremilast; Azathioprine; Baricitinib; Belimumab; Benralizumab; Bimekizumab; Brodalumab; Canakinumab; Certolizumab pegol; Cyclosporine; Dupilumab; erenumab-aooe; Estrasimod; Etanercept; Etanercept; Etrolizumab; Filgotinib; fremanezumab-vfrm; Galcanezumab-gnlm; Golimumab; Golimumab Aria; Guselkumab; Hydroxychloroquine; Infliximab; Infliximab; Interferon Beta 1a; Interferon Beta 1b; Ixekizumab; Lebrikizumab; Leflunomide; Mepolizumab; Methotrexate; Mirikizumab; Mycophenolate; Natalizumab; Ocrelizumab; Ofatumumab; Ozanimod; peginterferon beta-1a; Pegloticase; reslizumab; Risankizumab-rzaa; Rituximab; Sarilumab; Secukinumab; Sulfasalazine; Tezepelumab; Tildrakizumab; Tocilizumab; Tofacitinib; tralokinumab; Ublituximab; Upadacitinib; Ustekinumab; Vedolizumab. In some embodiments, the subject is taking one of the above-mentioned biological therapies. In other embodiments, the subject may be taking more than one of the above-mentioned biological therapies. In some embodiments, the subject has not taken one or more of the above-mentioned biological therapies.

### Administration and Dosing

Disclosed herein are methods and systems for estimating, using the patient-centric precision model disclosed herein, a dose and/or inter-dose interval for a biologic drug (e.g., an anti-TNF therapy) that will achieve disease remission of an immune-mediated inflammatory disease in a subject. In the case that the dose or inter-dose interval are above the maximum dose approved for treatment of the immune-mediated inflammatory disease with the biologic drug, then the patient-centric precision model disclosed herein, estimates that the subject will not respond to the biologic drug. In some embodiments, if the patient-centric precision model estimates that the subject will not respond to the biologic drug, the subject will be indicated for treatment with another therapeutic agent (other than the biologic drug), such as those disclosed elsewhere here.

In some embodiments, the subject is currently receiving a dose of a therapeutic agent disclosed herein. In some embodiments, the subject is prescribed a dose of a therapeutic agent disclosed herein. In some embodiments, the patient-centric precision model estimates a dose of a therapeutic agent disclosed herein. In some embodiments, the dose disclosed herein is expressed in a dosage range, a minimum dose, or a maximum dose. In some embodiments, the patient-centric precision model estimates an inter-dose interval for the therapeutic agent. In some embodiments, the inter-dose interval is elongated or shortened relative to an inter-dose interval of the therapeutic agent that the subject is currently receiving. In some embodiments, the inter-dose interval is elongated or shortened relative to an inter-dose interval of the therapeutic agent that was prescribed for the subject. In some embodiments, the inter-dose interval is elongated or shortened relative to an inter-dose interval of the therapeutic agent that is indicated for the subject on the label of the therapeutic agent. In some embodiments, the therapeutic agent is a biologic drug. In some embodiments, the biologic drug is or comprises an anti-TNF therapy. In some embodiments, the anti-TNF therapy comprises or is Infliximab or Adalimumab.

In some embodiments, methods comprise determining a dosage of a therapeutic agent disclosed herein, which is a pre-specified threshold based on a time to remission calculated, such as those provided, for example, in Table 1. In some embodiments, methods comprise determining a different therapeutic agent (for example, different from Infliximab), based on the time to remission calculated. In some embodiments, the drug administration may be an infusion cycle, or may be a dose administration.

In some embodiments, the dose of the therapeutic agent comprises about 2.5 milligrams per kilogram in body weight (mg/Kg), 5 mg/Kg, 7.5 mg/Kg, or 10 mg/Kg. In some embodiments, the dose of the therapeutic agent comprises from about 2.5 mg/Kg to about 10 mg/Kg. In some embodiments, the dose of the therapeutic agent comprises from about 2.5 mg/Kg to about 7.5 mg/Kg. In some embodiments, the dose of the therapeutic agent comprises from about 1 mg/Kg to about 7.5 mg/Kg. In some embodiments, inter-dose interval of the therapeutic agent comprises dosing every one day, one week, two weeks, three weeks, four weeks, five weeks, six weeks, seven weeks, eight weeks, nine weeks, ten weeks, eleven weeks, twelve weeks, thirteen weeks, fourteen weeks, fifteen weeks, sixteen weeks, seventeen weeks, eighteen week, nineteen weeks, or twenty weeks or more. In some embodiments, the therapeutic agent comprises or is Infliximab, or Adalimumab, or a combination thereof.

In some embodiments, the patient may be administered a biologic drug or small molecule, such as those described herein, through oral administration, inhalation, instillation, injection, sublingual or buccal administration, rectal administration, vaginal administration, or trans-dermal administration. In some embodiments, oral administration comprises a tablet, capsule, liquid, or mixtures thereof. In some embodiments, inhalation comprises an inhaler or nebulizer to administer the biologic drug or small molecule. In some embodiments, instillation comprises ocular, otic, or nasal administration of the biologic drug or small molecule. In some embodiments, injection comprises intravenous administration, intramuscular administration, intrathecal administration, or subcutaneous administration. In some embodiments, an injection may comprise an implantation by which a biologic drug or a small molecule can be released over a duration of time.

In some embodiments, the dose comprises 1 mg, 5 mg, 10 mg, 20 mg, 30 mg, 40 mg, 50 mg, 60 mg, 70 mg, 80 mg, 90 mg, 100 mg, 110 mg, or 120 mg dose or higher of a therapeutic agent. In some embodiments, the inter-dose interval comprises dosing every one day, one week, two weeks, three weeks, four weeks, five weeks, six weeks, seven weeks, eight weeks, nine weeks, ten weeks, eleven weeks, twelve weeks, thirteen weeks, fourteen weeks, fifteen weeks, sixteen weeks, seventeen weeks, eighteen week, nineteen weeks, or twenty weeks or more.

In some embodiments, the dose comprises about 1 mg to about 200 mg of the therapeutic agent. In some embodiments, the dose is or comprises about 1 mg to 5 mg, 1 mg to 10 mg, 1 mg to 20 mg, 1 mg to 30 mg, 1 mg to 40 mg, 1 mg to 50 mg, 1 mg to 60 mg, 1 mg to 70 mg, 1 mg to 80 mg, 1 mg to 90 mg, 1 mg to 100 mg, 1 mg to 110 mg, 1 mg to 120 mg, 1 mg to 130 mg, 1 mg to 140 mg, 1 mg to 150 mg, 1 mg to 160 mg, 1 mg to 162 mg, 1 mg to 165 mg, 1 mg to 170 mg, 1 mg to 180 mg, 1 mg to 190 mg, 1 mg to 200 mg, 5 mg to 10 mg, 5 mg to 20 mg, 5 mg to 30 mg, 5 mg to 40 mg, 5 mg to 50 mg, 5 mg to 60 mg, 5 mg to 70 mg, 5 mg to 80 mg, 5 mg to 90 mg, 5 mg to 100 mg, 5 mg to 110 mg, 5 mg to 120 mg, 5 mg to 130 mg, 5 mg to 140 mg, 5 mg to 150 mg, 5 mg to 160 mg, 5 mg to 162 mg, 5 mg to 165 mg, 5 mg to 170 mg, 5 mg to 180 mg, 5 mg to 190 mg, 5 mg to 200 mg, 10 mg to 20 mg, 10 mg to 30 mg, 10 mg to 40 mg, 10 mg to 50 mg, 10 mg to 60 mg, 10 mg to 70 mg, 10 mg to 80 mg, 10 mg to 90 mg, 10 mg to 100 mg, 10 mg to 110 mg, 10 mg to 120 mg, 10 mg to 130 mg, 10 mg to 140 mg, 10 mg to 150 mg, 10 mg to 160 mg, 10 mg to 162 mg, 10 mg to 165 mg, 10 mg to 170 mg, 10 mg to 180 mg, 10 mg to 190 mg, 10 mg to 200 mg, 20 mg to 30 mg, 20 mg to 40 mg, 20 mg to 50 mg, 20 mg to 60 mg, 20 mg to 70 mg, 20 mg to 80 mg, 20 mg to 90 mg, 20 mg to 100 mg, 20 mg to 110 mg, 20 mg to 120 mg, 20 mg to 130 mg, 20 mg to 140 mg, 20 mg to 150 mg, 20 mg to 160 mg, 20 mg to 162 mg, 20 mg to 165 mg, 20 mg to 170 mg, 20 mg to 180 mg, 20 mg to 190 mg, 20 mg to 200 mg, 30 mg to 40 mg, 30 mg to 50 mg, 30 mg to 60 mg, 30 mg to 70 mg, 30 mg to 80 mg, 30 mg to 90 mg, 30 mg to 100 mg, 30 mg to 110 mg, 30 mg to 120 mg, 30 mg to 130 mg, 30 mg to 140 mg, 30 mg to 150 mg, 30 mg to 160 mg, 30 mg to 162 mg, 30 mg to 165 mg, 30 mg to 170 mg, 30 mg to 180 mg, 30 mg to 190 mg, 30 mg to 200 mg, 40 mg to 50 mg, 40 mg to 60 mg, 40 mg to 70 mg, 40 mg to 80 mg, 40 mg to 90 mg, 40 mg to 100 mg, 40 mg to 110 mg, 40 mg to 120 mg, 40 mg to 130 mg, 40 mg to 140 mg, 40 mg to 150 mg, 40 mg to 160 mg, 40 mg to 162 mg, 40 mg to 165 mg, 40 mg to 170 mg, 40 mg to 180 mg, 40 mg to 190 mg, 40 mg to 200 mg, 50 mg to 60 mg, 50 mg to 70 mg, 50 mg to 80 mg, 50 mg to 90 mg, 50 mg to 100 mg, 50 mg to 110 mg, 50 mg to 120 mg, 50 mg to 130 mg, 50 mg to 140 mg, 50 mg to 150 mg, 50 mg to 160 mg, 50 mg to 162 mg, 50 mg to 165 mg, 50 mg to 170 mg, 50 mg to 180 mg, 50 mg to 190 mg, 50 mg to 200 mg, 60 mg to 70 mg, 60 mg to 80 mg, 60 mg to 90 mg, 60 mg to 100 mg, 60 mg to 110 mg, 60 mg to 120 mg, 60 mg to 130 mg, 60 mg to 140 mg, 60 mg to 150 mg, 60 mg to 160 mg, 60 mg to 162 mg, 60 mg to 165 mg, 60 mg to 170 mg, 60 mg to 180 mg, 60 mg to 190 mg, 60 mg to 200 mg, 70 mg to 80 mg, 70 mg to 90 mg, 70 mg to 100 mg, 70 mg to 110 mg, 70 mg to 120 mg, 70 mg to 130 mg, 70 mg to 140 mg, 70 mg to 150 mg, 70 mg to 160 mg, 70 mg to 162 mg, 70 mg to 165 mg, 70 mg to 170 mg, 70 mg to 180 mg, 70 mg to 190 mg, 70 mg to 200 mg, 80 mg to 90 mg, 80 mg to 100 mg, 80 mg to 110 mg, 80 mg to 120 mg, 80 mg to 130 mg, 80 mg to 140 mg, 80 mg to 150 mg, 80 mg to 160 mg, 80 mg to 162 mg, 80 mg to 165 mg, 80 mg to 170 mg, 80 mg to 180 mg, 80 mg to 190 mg, 80 mg to 200 mg, 90 mg to 100 mg, 90 mg to 110 mg, 90 mg to 120 mg, 90 mg to 130 mg, 90 mg to 140 mg, 90 mg to 150 mg, 90 mg to 160 mg, 90 mg to 162 mg, 90 mg to 165 mg, 90 mg to 170 mg, 90 mg to 180 mg, 90 mg to 190 mg, 90 mg to 200 mg, 100 mg to 110 mg, 100 mg to 120 mg, 100 mg to 130 mg, 100 mg to 140 mg, 100 mg to 150 mg, 100 mg to 160 mg, 100 mg to 162 mg, 100 mg to 165 mg, 100 mg to 170 mg, 100 mg to 180 mg, 100 mg to 190 mg, 100 mg to 200 mg, 110 mg to 120 mg, 110 mg to 130 mg, 110 mg to 140 mg, 110 mg to 150 mg, 110 mg to 160 mg, 110 mg to 162 mg, 110 mg to 165 mg, 110 mg to 170 mg, 110 mg to 180 mg, 110 mg to 190 mg, 110 mg to 200 mg, 120 mg to 130 mg, 120 mg to 140 mg, 120 mg to 150 mg, 120 mg to 160 mg, 120 mg to 162 mg, 120 mg to 165 mg, 120 mg to 170 mg, 120 mg to 180 mg, 120 mg to 190 mg, 120 mg to 200 mg, 130 mg to 140 mg, 130 mg to 150 mg, 130 mg to 160 mg, 130 mg to 162 mg, 130 mg to 165 mg, 130 mg to 170 mg, 130 mg to 180 mg, 130 mg to 190 mg, 130 mg to 200 mg, 140 mg to 150 mg, 140 mg to 160 mg, 140 mg to 162 mg, 140 mg to 165 mg, 140 mg to 170 mg, 140 mg to 180 mg, 140 mg to 190 mg, 140 mg to 200 mg, 150 mg to 160 mg, 150 mg to 162 mg, 150 mg to 165 mg, 150 mg to 170 mg, 150 mg to 180 mg, 150 mg to 190 mg, 150 mg to 200 mg, 160 mg to 162 mg, 160 mg to 165 mg, 160 mg to 170 mg, 160 mg to 180 mg, 160 mg to 190 mg, 160 mg to 200 mg, 162 mg to 165 mg, 162 mg to 170 mg, 162 mg to 180 mg, 162 mg to 190 mg, 162 mg to 200 mg, 165 mg to 170 mg, 165 mg to 180 mg, 165 mg to 190 mg, 165 mg to 200 mg, 170 mg to 180 mg, 170 mg to 190 mg, 170 mg to 200 mg, 180 mg to 190 mg, 180 mg to 200 mg, or 190 mg to 200 mg of the therapeutic agent. In some embodiments, the dose is or comprises about 1 mg, 5 mg, 10 mg, 20 mg, 30 mg, 40 mg, 50 mg, 60 mg, 70, mg, 80 mg, 90 mg, 100 mg, 110 mg, 120 mg, 130 mg, 140 mg, 150 mg, 160 mg, 162 mg, 165 mg, 170 mg, 180 mg, 190 mg, or 200 mg of the therapeutic agent. In some embodiments, the dose is or comprises at least about 1 mg, 5 mg, 10 mg, 20 mg, 30 mg, 40 mg, 50 mg, 60 mg, 70, mg, 80 mg, 90 mg, 100 mg, 110 mg, 120 mg, 130 mg, 140 mg, 150 mg, 160 mg, 162 mg, 165 mg, 170 mg, 180 mg, or 190 mg of the therapeutic agent. In some embodiments, the dose is or comprises about 5 mg, 10 mg, 20 mg, 30 mg, 40 mg, 50 mg, 60 mg, 70, mg, 80 mg, 90 mg, 100 mg, 110 mg, 120 mg, 130 mg, 140 mg, 150 mg, 160 mg, 162 mg, 165 mg, 170 mg, 180 mg, 190 mg, or 200 mg of the therapeutic agent.

In some embodiments, the dose is or comprises 0.5 mg/kg to about 30 mg/kg of the therapeutic agent. In some embodiments, the dose is or comprises about 0.5 mg/kg to about 1 mg/kg, about 0.5 mg/kg to about 5 mg/kg, about 0.5 mg/kg to about 10 mg/kg, about 0.5 mg/kg to about 15 mg/kg, about 0.5 mg/kg to about 20 mg/kg, about 0.5 mg/kg to about 25 mg/kg, about 0.5 mg/kg to about 30 mg/kg, about 1 mg/kg to about 5 mg/kg, about 1 mg/kg to about 10 mg/kg, about 1 mg/kg to about 15 mg/kg, about 1 mg/kg to about 20 mg/kg, about 1 mg/kg to about 25 mg/kg, about 1 mg/kg to about 30 mg/kg, about 5 mg/kg to about 10 mg/kg, about 5 mg/kg to about 15 mg/kg, about 5 mg/kg to about 20 mg/kg, about 5 mg/kg to about 25 mg/kg, about 5 mg/kg to about 30 mg/kg, about 10 mg/kg to about 15 mg/kg, about 10 mg/kg to about 20 mg/kg, about 10 mg/kg to about 25 mg/kg, about 10 mg/kg to about 30 mg/kg, about 15 mg/kg to about 20 mg/kg, about 15 mg/kg to about 25 mg/kg, about 15 mg/kg to about 30 mg/kg, about 20 mg/kg to about 25 mg/kg, about 20 mg/kg to about 30 mg/kg, or about 25 mg/kg to about 30 mg/kg of the therapeutic agent. In some embodiments, the dose is or comprises about 0.5 mg/kg, about 1 mg/kg, about 5 mg/kg, about 10 mg/kg, about 15 mg/kg, about 20 mg/kg, about 25 mg/kg, or about 30 mg/kg of the therapeutic agent. In some embodiments, the dose is or comprises at least about 0.5 mg/kg, about 1 mg/kg, about 5 mg/kg, about 10 mg/kg, about 15 mg/kg, about 20 mg/kg, or about 25 mg/kg of the therapeutic agent. In some embodiments, the dose is or comprises at most about 1 mg/kg, about 5 mg/kg, about 10 mg/kg, about 15 mg/kg, about 20 mg/kg, about 25 mg/kg, or about 30 mg/kg of the therapeutic agent.

In some embodiments, the inter-dose interval is or comprises about twice a week to about every sixteen weeks. In some embodiments, the inter-dose interval is or comprises about twice a week to about one week, about twice a week to about two weeks, about twice a week to about three weeks, about twice a week to about four weeks, about twice a week to about five weeks, about twice a week to about six weeks, about twice a week to about seven weeks, about twice a week to about eight weeks, about twice a week to about nine weeks, about twice a week to about ten weeks, about twice a week to about eleven weeks, about twice a week to about twelve weeks, about twice a week to about thirteen weeks, about twice a week to about fourteen weeks, about twice a week to about fifteen weeks, about twice a week to about sixteen weeks, about one week to about two weeks, about one week to about three weeks, about one week to about four weeks, about one week to about five weeks, about one week to about six weeks, about one week to about seven weeks, about one week to about eight weeks, about one week to about nine weeks, about one week to about ten weeks, about one week to about eleven weeks, about one week to about twelve weeks, about one week to about thirteen weeks, about one week to about fourteen weeks, about one week to about fifteen weeks, about one week to about sixteen weeks, about two weeks to about three weeks, about two weeks to about four weeks, about two weeks to about five weeks, about two weeks to about six weeks, about two weeks to about seven weeks, about two weeks to about eight weeks, about two weeks to about nine weeks, about two weeks to about ten weeks, about two weeks to about eleven weeks, about two weeks to about twelve weeks, about two weeks to about thirteen weeks, about two weeks to about fourteen weeks, about two weeks to about fifteen weeks, about two weeks to about sixteen weeks, about three weeks to about four weeks, about three weeks to about five weeks, about three weeks to about six weeks, about three weeks to about seven weeks, about three weeks to about eight weeks, about three weeks to about nine weeks, about three weeks to about ten weeks, about three weeks to about eleven weeks, about three weeks to about twelve weeks, about three weeks to about thirteen weeks, about three weeks to about fourteen weeks, about three weeks to about fifteen weeks, about three weeks to about sixteen weeks, about four weeks to about five weeks, about four weeks to about six weeks, about four weeks to about seven weeks, about four weeks to about eight weeks, about four weeks to about nine weeks, about four weeks to about ten weeks, about four weeks to about eleven weeks, about four weeks to about twelve weeks, about four weeks to about thirteen weeks, about four weeks to about fourteen weeks, about four weeks to about fifteen weeks, about four weeks to about sixteen weeks, about five weeks to about six weeks, about five weeks to about seven weeks, about five weeks to about eight weeks, about five weeks to about nine weeks, about five weeks to about ten weeks, about six weeks to about seven weeks, about six weeks to about eight weeks, about six weeks to about nine weeks, about six weeks to about ten weeks, about six weeks to about eleven weeks, about six weeks to about twelve weeks, about six weeks to about thirteen weeks, about six weeks to about fourteen weeks, about six weeks to about fifteen weeks, about six weeks to about sixteen weeks, about seven weeks to about eight weeks, about seven weeks to about nine weeks, about seven weeks to about ten weeks, about seven weeks to about eleven weeks, about seven weeks to about twelve weeks, about seven weeks to about thirteen weeks, about seven weeks to about fourteen weeks, about seven weeks to about fifteen weeks, about seven weeks to about sixteen weeks, about eight weeks to about nine weeks, about eight weeks to about ten weeks, about eight weeks to about eleven weeks, about eight weeks to about twelve weeks, about eight weeks to about thirteen weeks, about eight weeks to about fourteen weeks, about eight weeks to about fifteen weeks, about eight weeks to about sixteen weeks, about nine weeks to about ten weeks, about nine weeks to about eleven weeks, about nine weeks to about twelve weeks, about nine weeks to about thirteen weeks, about nine weeks to about fourteen weeks, about nine weeks to about fifteen weeks, about nine weeks to about sixteen weeks, about ten weeks to about eleven weeks, about ten weeks to about twelve weeks, about ten weeks to about thirteen weeks, about ten weeks to about fourteen weeks, about ten weeks to about fifteen weeks, about ten weeks to about sixteen weeks, about eleven weeks to about twelve weeks, about eleven weeks to about thirteen weeks, about eleven weeks to about fourteen weeks, about eleven weeks to about fifteen weeks, about eleven weeks to about sixteen weeks, about twelve weeks to about thirteen weeks, about twelve weeks to about fourteen weeks, about twelve weeks to about fifteen weeks, about twelve weeks to about sixteen weeks, about thirteen weeks to about fourteen weeks, about thirteen weeks to about fifteen weeks, about thirteen weeks to about sixteen weeks, about fourteen weeks to about fifteen weeks, about fourteen weeks to about sixteen weeks, or about fifteen weeks to about sixteen weeks. In some embodiments, the inter-dose interval is or comprises about twice a week, about one week, about two weeks, about three weeks, about four weeks, about five weeks, about six weeks, about seven weeks, about eight weeks, about nine weeks, about ten weeks, about eleven weeks, about twelve weeks, about thirteen weeks, about fourteen weeks, about fifteen weeks, or about sixteen weeks. In some embodiments, the inter-dose interval is or comprises at least about twice a week, about one week, about two weeks, about three weeks, about four weeks, about five weeks, about six weeks, about seven weeks, about eight weeks, about ten weeks, about eleven weeks, about twelve weeks, about thirteen weeks, about fourteen weeks, or about fifteen weeks. In some embodiments, the inter-dose interval is or comprises at most about one week, about two weeks, about three weeks, about four weeks, about five weeks, about six weeks, about seven weeks, about eight weeks, about nine weeks, about ten weeks, about eleven weeks, about twelve weeks, about thirteen weeks, about fourteen weeks, about fifteen weeks, or about sixteen weeks.

In some embodiments, the dose of the therapeutic agent is about 40 mg and the inter-dose interval is every two weeks. In some embodiments, the dose of the therapeutic agent is about 20 mg to about 80 mg, and the inter-dose interval is every week to every six weeks. In some embodiments, the maximum dose of the therapeutic agent is 80 mg and the minimum inter-dose interval is weekly. In some embodiments, the maximum dose amount is 80 mg. In some embodiments, the therapeutic agent is or comprises ADA.

In some embodiments, the dose of the therapeutic agent is about 5 mg/kg and the inter-dose interval is every eight weeks. In some embodiments, the dose of the therapeutic agent is about 3 mg/kg to about 15 mg/kg, and the inter-dose level is every four weeks to every twelve weeks. In some embodiments, the maximum dose of the therapeutic agent is about 15 mg/kg and the minimum inter-dose interval is every four weeks. In some embodiments, the maximum dose amount is 15 mg/kg. In some embodiments, the therapeutic agent is or comprises IFX.

### NUMBERED EMBODIMENTS

1. A method for treating an immune-mediated inflammatory disease in a subject, said method comprising: administering a dose of a biologic drug to the subject to treat the immune-mediated inflammatory disease, wherein the subject is selected based on a combined poor prognosis factor (PPF) score below 3 on a scale of 0 to 3 calculated using a statistical model, wherein the combined PPF score reflects a clearance estimate of the biologic drug and a presence of a genotype at a single nucleotide variant (SNV) comprising rs2097432 or a SNV in linkage disequilibrium therewith detected in a biological sample obtained from the subject.
2. A method for treating an immune-mediated inflammatory disease in a subject, said method comprising: administering a dose of an immune-mediated inflammatory disease therapy other than an biologic drug to the subject to treat the immune-mediated inflammatory disease, wherein the subject was selected based on combined poor prognosis factor (PPF) score of 3 on a scale of 0 to 3 calculated using a statistical model, wherein the combined PPF score reflects a clearance estimate of the biologic drug and a presence of a genotype at a single nucleotide variant (SNV) comprising rs2097432 or a SNV in linkage disequilibrium therewith detected in a biological sample obtained from the subject.
3. The method of embodiment 1 or 2, wherein the statistical model comprises one or more algorithms comprising a Naïve Bayes classifier algorithm, a non-linear mixed effects model (NLME), or a Metropolis Hastings algorithm, or any combination thereof.
4. The method of embodiment 1 or 2, wherein the statistical model predicts a time to remission for the subject of the immune-mediated inflammatory disease.
5. The method of embodiment 4, wherein the statistical model predicts the time to remission for the subject by a method comprising:
   (a) receiving data from a database, wherein the data is related to disease remission in individuals from a reference population having the immune-mediated inflammatory disease that have been treated with the biologic drug;
   (b) establishing a first set of parameter estimates from the data;
   (c) deriving a second set of parameter estimates for the statistical model based at least in part on the first set of parameter estimates;
   (d) receiving subject specific data comprising: (i) the genotype at the SNV comprising rs2097432 or the SNV in linkage disequilibrium therewith; (ii) a weight or body mass index (BMI) of the subject; and (iii) a level of albumin measured in the biological sample;
   (e) updating the model based at least in part on the subject specific data received in (d); and
   (f) determining the time to remission for the subject, wherein the time to remission is reflected in a combined PPF score on the scale of 0 to 3.
6. The method of embodiment 5, wherein the subject specific data further comprises a level of c-reactive protein (CRP), wherein a CRP above 3 milligrams per liter (mg/L) is indicative of a presence of inflammation in the subject.
7. The method of embodiment 5, wherein the subject specific data further comprises a level of autoantibodies against the biologic drug, a level of the biologic drug, or a combination thereof.
8. The method of embodiment 1 of 2, wherein the combined PPF score further reflects a wellbeing of the subject based on patient wellbeing data received by the structural model.
9. The method of embodiment 8, wherein the patient wellbeing data comprises a score on a disease activity index for the immune-mediated inflammatory disease.
10. The method of embodiment 9, wherein the disease activity index is Crohn's Disease Activity Index (CDAI) when the immune-mediated inflammatory disease is Crohn's disease.
11. The method of embodiment 9, wherein the disease activity index is a Mayo Clinical Score when the immune-mediated inflammatory disease is Ulcerative Colitis.
12. The method of embodiment of embodiment 8, wherein the patient wellbeing data comprises symptoms of the immune-mediated inflammatory disease, wherein the symptoms comprise diarrhea, abdominal pain, abdominal cramping, loss of appetite, weight loss, fatigue, fever, rectal bleeding, a skin rash, joint pain, stiffness, or swelling, decreased range of motion for joints, redness at joints, or any combination thereof.
13. The method of embodiment 8, wherein the patient wellbeing data comprises body mass index (BMI) or weight.
14. A method for treating an immune-mediated inflammatory disease in a subject, said method comprising:
   (a) calculating a poor prognosis factor 1 (PPF1) score for the subject comprising:
      (i) obtaining or having obtained a biological sample from the subject;
      (ii) measuring a level of albumin in the biological sample;
      (iii) calculating a rate of which an biologic drug is estimated to be cleared from the subject using a first statistical model based, at least in part, on the level of the albumin measured in the biological sample and a weight of the subject; and
      (iv) comparing the rate to a predetermined threshold for the biologic drug, to produce an estimated clearance score on a scale of 0 to 1, wherein 0 is indicative of a low rate of clearance below the predetermined threshold, and 1 is indicative of a high rate of clearance above the predetermined threshold;
   (b) calculating a poor prognosis factor 2 (PPF 2) score for the subject, comprising:
      (i) obtaining or having obtained the biological sample from the subject;
      (ii) performing or having performed a genotyping assay on the biological sample to detect a genotype at single nucleotide variant (SNV) comprising rs2097432 or a SNV in linkage disequilibrium therewith; and
      (iii) calculating a PPF2 score using a second statistical model, wherein the PPF2 score is on a scale of 0 to 2, wherein 0 is indicative of the genotype that is homozygous non-risk, 1 is indicative of the genotype that is heterozygous risk at rs2097432, and 2 comprises a presence of two genetic loci of said genotype;
   (c) calculating a poor prognosis factor 4 (PPF 4) for the subject comprising:
      (i) receiving wellbeing data from said subject comprising one or more symptoms related to the immune-mediated inflammatory disease;
      (ii) calculating a wellbeing score with a fourth statistical model of 0 or 1 based on the wellbeing data received, wherein 0 is indicative of an improvement of at least one symptom of the one or more symptoms, and 1 is indicative of no improvement in the at least one symptom of the one or more symptoms; and
   (d) calculating a combined PPF score comprising the PPF1 score, the PPF2 score, and the PPF4 score to calculate a percentage reduction in hazard remission
15. The method of embodiment 14, further comprising calculating a poor prognosis factor 3 (PPF 3) score for the subject, comprising:
   (i) obtaining or having obtained the biological sample from the subject;
   (ii) performing or having performed an immunological assay on the biological sample to detect a level of a serological marker; and
   (iii) calculating a PPF3 score using a third statistical model, wherein the PPF3 score is on a scale of 0 to 2, wherein 0 is indicative of a standard response to the biologic drug, 1 is indicative e of a low response to the biologic drug, and 2 is indicative of very low response to the biologic drug.
16. The method of embodiment 15, wherein the serological marker comprises perinuclear antineutrophil cytoplasmic antibodies (pANCA), proteinase 3 (PR3), or one or more antibodies against an integrin.
17. The method of any one of embodiments 14-16, wherein the predetermined threshold comprises about 0.2 to 0.4 L/day of the biologic drug.
18. The method of any one of embodiments 14-16, wherein the predetermined threshold is above about 0.3 L/day of the biologic drug.
19. The method of any one of embodiments 14-18, wherein the biologic drug is an anti-TNF therapy, wherein the anti-TNF therapy is optionally Infliximab.
20. The method of embodiment 19, wherein the percentage reduction in hazard remission above 78%.
21. The method of embodiment 20, wherein the percentage in hazard remission above 78% is indicative that the subject should receive a first dose of the biologic drug, such as for example, 5 milligrams per kilogram (mg/Kg) of Infliximab.
22. The method of any one of embodiments 14-18, wherein the percentage reduction in hazard remission comprising from 56% to 78%.
23. The method of embodiment 22, wherein the percentage reduction in hazard remission comprising from 56% to 78% % is indicative that the subject should receive a second dose of the biologic drug that is larger than the first dose, such as for example, 7.5 mg/Kg of Infliximab.
24. The method of any one of embodiments 14-18, wherein the percentage reduction in hazard remission comprises from 34 to 56%.
25. The method of embodiment 24, wherein the percentage reduction in hazard remission comprising from 34 to 56% is indicative that the subject should receive a third dose of the biologic drug that is larger than the first dose and second dose, such as for example, 10 mg/Kg of Infliximab.
26. The method of any one of embodiments 14-18, wherein the percentage reduction in hazard remission comprises 34% or less.
27. The method of embodiment 26, wherein the percentage reduction in hazard remission comprising 34% or less is indicative that the subject should receive a therapy other than the biologic drug, such as for example, a small molecule Janus Kinase (JAK) or a small molecule modulator of sphingosine 1-phosphate (S1P).
28. The method of embodiment 25, wherein the small molecule inhibitor of JAK comprises Tofacitinib.
29. The method of embodiment 27, wherein the small molecule modulator of S1P comprises Ozanimod.
30. The method of any one of embodiments 14-29, wherein the first statistical model, the second statistical model, the third statistical model, or the fourth statistical model comprises one or more algorithms comprising a Naïve Bayes classifier algorithm, a non-linear mixed effects model (NLME), or a Metropolis Hastings algorithm, or any combination thereof.
31. The method of any one of embodiments 1-30, wherein the biologic drug comprises infliximab, adalimumab, etanercept, golimumab, certolizumab, ustekinumab, vedolizumab, tocilizumab, an inhibitor of interleukin 6 (e.g., Tocilizumab), or an inhibitor of tumor necrosis factor ligand 1 (TL1A).
32. The method of any one of embodiments 1-30, wherein the immune-mediated inflammatory disease comprises an inflammatory bowel disease (IBD), rheumatoid arthritis (RA), cytokine release syndrome, multiple sclerosis (MS), ankylosing spondylitis (AS), lupus, plaque psoriasis, atopic dermatitis, gout, migraine, cancer, or a neoplasm.
33. The method of embodiment 32, wherein the IBD comprises Crohn's disease.
34. The method of embodiment 32, wherein the IBD comprises Ulcerative Colitis.
35. The method of any one of embodiments 1-34, wherein the biological sample obtained from the subject is blood serum.
36. The method of any one of embodiments 1-35, wherein the genotype at the SNV comprising rs2097432 comprises a “G.” 37. The method of embodiment 36, wherein the genotype at the SNV comprising s2097432 is homozygous.
38. The method of any one of embodiments 1-37, wherein the linkage disequilibrium is determined with an r² of at least about 0.84.
39. The method of any one of embodiments 1-38, wherein the subject is age 18 or older.
40. A computer-implemented system comprising a computing device comprising at least one processor, an operating system configured to perform executable instructions, a memory, and a computer program including instructions executable by the computing device to perform a method comprising:
   (a) calculating a poor prognosis factor 1 (PPF1) score for the subject comprising:
      (i) receiving a level of albumin measured in a biological sample obtained from a subject receiving biologic drug for treatment of a immune-mediated inflammatory disease, and a weight of the subject;
      (iii) calculating a rate of which the biologic drug is estimated to be cleared from the subject using a first statistical model based, at least in part, on the level of the albumin measured in the biological sample, and the weight of the subject; and
      (iv) comparing the rate to a predetermined threshold for the biologic drug, to produce an estimated clearance score on a scale of 0 to 1, wherein 0 is indicative of a low rate of clearance below the predetermined threshold, and 1 is indicative of a high rate of clearance above the predetermined threshold;
   (b) calculating a poor prognosis factor 2 (PPF 2) score for the subject comprising:
      (i) receiving a genotype of the subject at a single nucleotide variant (SNV) comprising rs2097432 or a SNV in linkage disequilibrium therewith; and
      (iii) calculating a PPF2 score using a second statistical model, wherein the PPF2 score is on a scale of 0 to 2, wherein 0 is indicative of the genotype that is homozygous non-risk, 1 is indicative of the genotype that is heterozygous risk at rs2097432, and 2 comprises a presence of two genetic loci of said genotype; and
   (c) calculating a combined PPF score comprising the PPF1 score and the PPF2 score, to predict a time to remission following administration of the biologic drug to the subject.
41. The system of embodiment 40, wherein the method further comprises: (d) calculating a poor prognosis factor 3 (PPF 3) score for the subject, comprising:
   (i) receiving a level of a serological marker detected in the biological sample obtained from the subject; and
   (ii) calculating a PPF3 score using a third statistical model, wherein the PPF3 score is on a scale of 0 to 2, wherein 0 is indicative of a standard response to the biologic drug, 1 is indicative e of a low response to the biologic drug, and 2 is indicative of very low response to the biologic drug.
42. The system of embodiment 40 or 41, wherein the serological marker comprises perinuclear antineutrophil cytoplasmic antibodies (pANCA), proteinase 3 (PR3), or one or more antibodies against an integrin, wherein, optionally:
   a. a PFF3 score of 0 is indicative of an absence of the pANCA and a level of PR3 that is within the 99^{th} percentile with reference to a reference population of individuals that do not have an immune-mediated inflammatory disease;
   b. a PPF3 core of 2 is indicative of a presence of pANCA and a level of PR3 that is above the 99^{th} percentile; and
   c. a PPF3 score of 1 is indicative of either the presence of pANCA or the level of PR3 that is above the 99^{th} percentile.
43. The system of embodiment 42, wherein the method further comprises: (e) calculating a poor prognosis factor 4 (PPF 4) for the subject comprising:
   (i) receiving wellbeing data from the subject comprising one or more symptoms related to the immune-mediated inflammatory disease;
   (ii) calculating a wellbeing score with a fourth statistical model of 0 or 1 based on the wellbeing data received, wherein 0 is indicative of an improvement of at least one symptom of the one or more symptoms, and 1 is indicative of no improvement in the at least one symptom of the one or more symptoms, wherein the combined PPF score calculated in (c) further comprises the PPF score calculated in (d).
44. The system of any one of embodiments 40-43, wherein the biologic drug comprises Infliximab, Adalimumab, Etanercept, Golimumab, or Certolizumab, Ustekinumab, Vedolizumab, Tocilizumab, an inhibitor of interleukin 6 (e.g., Tocilizumab), or an inhibitor of TL1A.
45. The system of any one of embodiments 40-44, wherein the immune-mediated inflammatory disease comprises an inflammatory bowel disease (IBD), rheumatoid arthritis (RA), cytokine release syndrome, multiple sclerosis (MS), ankylosing spondylitis (AS), lupus, plaque psoriasis, atopic dermatitis, gout, migraine, cancer, or a neoplasm.
46. The system of embodiment 45, wherein the IBD comprises Crohn's disease.
47. The system of embodiment 45, wherein the IBD comprises Ulcerative Colitis.
48. The system of any one of embodiments 40-47, further comprising the biological sample, wherein the biological sample is blood serum.
49. The system of any one of embodiments 40-48, wherein the genotype at the SNV comprising rs2097432 comprises a “G.”
50. The system of embodiment 49, wherein the genotype at the SNV comprising rs2097432 is homozygous.
51. The system of any one of embodiments 40-50, wherein the linkage disequilibrium is determined with an r² of at least about 0.84.
52. The system of any one of embodiments 40-51, wherein the subject is age 18 or older.
53. The system of any one of embodiments 40-52, wherein the predetermined threshold comprises about 0.2 to 0.4 L/day of the biologic drug.
54. The system of any one of embodiments 40-52, wherein the predetermined threshold is above about 0.3 L/day of the biologic drug.
55. The system of any one of embodiments 40-53, wherein the method further comprising determining a percentage reduction in hazard remission based on the time to remission.
56. The system of embodiment 55, wherein if the percentage reduction in hazard remission is above 34%, then the subject is indicated for treatment with the biologic drug.
57. The system of embodiment 55, wherein if the percentage reduction in hazard remission is above 78%, then the subject is indicated for treatment with a first dose of the biological drug, such as for example, 5 mg/Kg of Infliximab.
58. The system of embodiment 55, wherein if the percentage reduction in hazard remission comprises from 56% to 78%, then the subject is indicated for treatment with a second dose of the biological drug that is larger than the first dose, such as for example, 7.5 mg/Kg of Infliximab.
59. The system of embodiment 55, wherein if the percentage reduction in hazard remission comprises from 34% to 56%, then the subject is indicated for treatment with a third dose of the biological drug that is larger than the first dose and the second dose, such as for example, 10 mg/Kg of Infliximab.
60. The system of embodiment 55, wherein if the percentage reduction in hazard remission is 34% or below, then the subject is indicated for treatment with a therapy other than the biologic drug, such as for example, a small molecule inhibitor of JAK or modulator of S1P.
61. A non-transitory computer readable storage medium encoded with a computer program including instructions executable by one or more processors to statistic model for predicting disease remission in a subject with an immune-mediated inflammatory disease, the computer program configured to cause the one or more processors to perform a method comprising
   (a) calculating a poor prognosis factor 1 (PPF1) score for the subject comprising:
      (i) receiving a level of albumin measured in a biological sample obtained from a subject receiving biologic drug for treatment of a immune-mediated inflammatory disease, and a weight of the subject;
      (ii) calculating a rate of which the biologic drug is estimated to be cleared from the subject using a first statistical model based, at least in part, on the level of the albumin measured in the biological sample, and the weight of the subject; and
      (iii) comparing the rate to a predetermined threshold for the biologic drug, to produce an estimated clearance score on a scale of 0 to 1, wherein 0 is indicative of a low rate of clearance below the predetermined threshold, and 1 is indicative of a high rate of clearance above the predetermined threshold;
   (b) calculating a poor prognosis factor 2 (PPF 2) score for the subject comprising:
      (i) receiving a genotype of the subject at a single nucleotide variant (SNV) comprising rs2097432 or a SNV in linkage disequilibrium therewith; and
      (ii) calculating a PPF2 score using a second statistical model, wherein the PPF2 score is on a scale of 0 to 2, wherein 0 is indicative of the genotype that is homozygous non-risk, 1 is indicative of the genotype that is heterozygous risk at rs2097432, and 2 comprises a presence of two genetic loci of said genotype; and
   (c) calculating a combined PPF score comprising the PPF1 score, the PPF2 score, and the PPF3 score to predict a time to remission following administration of the biologic drug to the subject.
62. The non-transitory computer-readable storage media of embodiment 61, wherein the method further comprises: (d) calculating a poor prognosis factor 3 (PPF 3) score for the subject, comprising:
   (i) receiving a level of a serological marker detected in the biological sample obtained from the subject; and
   (ii) calculating a PPF3 score using a third statistical model, wherein the PPF3 score is on a scale of 0 to 2, wherein 0 is indicative of a standard response to the biologic drug, 1 is indicative e of a low response to the biologic drug, and 2 is indicative of very low response to the biologic drug.
63. The non-transitory computer-readable storage media of embodiment 62, wherein the serological marker comprises perinuclear antineutrophil cytoplasmic antibodies (pANCA), proteinase 3 (PR3), or one or more antibodies against an integrin, wherein, optionally:
   a. a PFF3 score of 0 is indicative of an absence of the pANCA and a level of PR3 that is within the 99^{th} percentile with reference to a reference population of individuals that do not have an immune-mediated inflammatory disease;
   b. a PPF3 core of 2 is indicative of a presence of pANCA and a level of PR3 that is above the 99^{th} percentile; and
   c. a PPF3 score of 1 is indicative of either the presence of pANCA or the level of PR3 that is above the 99^{th} percentile.
64. The non-transitory computer-readable storage media of any one of embodiments 61-63, wherein the method further comprises:
   (e) calculating a poor prognosis factor 4 (PPF 4) for the subject comprising:
      (i) receiving wellbeing data from the subject comprising one or more symptoms related to the immune-mediated inflammatory disease;
      (ii) calculating a wellbeing score with a fourth statistical model of 0 or 1 based on the wellbeing data received, wherein 0 is indicative of an improvement of at least one symptom of the one or more symptoms, and 1 is indicative of no improvement in the at least one symptom of the one or more symptoms, wherein the combined PPF score calculated in (c) further comprises the PPF score calculated in (d).
65. The non-transitory computer-readable storage media of any one of embodiments 61-64, wherein the biologic drug comprises Infliximab, adalimumab, etanercept, golimumab, or certolizumab.
66. The non-transitory computer-readable storage media of any one of embodiments 61-65, wherein the immune-mediated inflammatory disease comprises an inflammatory bowel disease (IBD), rheumatoid arthritis (RA), cytokine release syndrome, multiple sclerosis (MS), ankylosing spondylitis (AS), lupus, plaque psoriasis, atopic dermatitis, gout, migraine, cancer, or a neoplasm.
67. The non-transitory computer-readable storage media of embodiment 66, wherein the IBD comprises Crohn's disease.
68. The non-transitory computer-readable storage media of embodiment 66, wherein the IBD comprises Ulcerative Colitis.
69. The non-transitory computer-readable storage media of any one of embodiments 61-68, wherein the biological sample is blood serum.
70. The non-transitory computer-readable storage media of any one of embodiments 61-69, wherein the genotype at the SNV comprising rs2097432 comprises a “G.”
71. The non-transitory computer-readable storage media of embodiment 70, wherein the genotype at the SNV comprising s2097432 is homozygous.
72. The non-transitory computer-readable storage media of any one of embodiments 61-71, wherein the linkage disequilibrium is determined with an r² of at least about 0.84.
73. The non-transitory computer-readable storage media of any one of embodiments 61-72, wherein the subject is age 18 or older.
74. The non-transitory computer-readable storage media of any one of embodiments 61-73, wherein the predetermined threshold comprises about 0.2 to 0.4 L/day of the biologic drug.
75. The non-transitory computer-readable storage media of any one of embodiments 61-73, wherein the predetermined threshold is above about 0.3 L/day of the biologic drug.
76. The non-transitory computer-readable storage media of any one of embodiments 61-74, wherein the method further comprising determining a percentage reduction in hazard remission based on the time to remission.
77. The non-transitory computer-readable storage media of embodiment 76, wherein if the percentage reduction in hazard remission is above 34%, then the subject is indicated for treatment with the biologic drug, such as for example, Infliximab.
78. The non-transitory computer-readable storage media of embodiment 76, wherein if the percentage reduction in hazard remission is above 78%, then the subject is indicated for treatment with a first dose of the biologic drug, such as for example 5 mg/Kg of Infliximab.
79. The non-transitory computer-readable storage media of embodiment 76, wherein if the percentage reduction in hazard remission comprises from 56% to 78%, then the subject is indicated for treatment with a second dose of the biologic drug that is larger than the first dose, such as for example, 7.5 mg/Kg of Infliximab.
80. The non-transitory computer-readable storage media of embodiment 76, wherein if the percentage reduction in hazard remission comprises from 34% to 56%, then the subject is indicated for treatment with a third dose of the biologic drug that is larger than the first dose and second dose, such as for example, 10 mg/Kg of Infliximab.
81. The non-transitory computer-readable storage media of embodiment 76, wherein if the percentage reduction in hazard remission is 34% or below, then the subject is indicated for treatment with a therapy other than the biologic drug.
82. A method of predicting whether an immune-mediated inflammatory disease in a subject will positively respond to a treatment with an biologic drug, the method comprising:
   (a) calculating an estimated clearance rate at which an biologic drug is cleared from the subject using a first statistical model based, at least in part, on a level of albumin measured in a biological sample obtained from the subject, and a weight of the subject;
   (b) comparing the clearance rate calculated in (a) to a predetermined threshold, to produce poor prognosis factor 1 (PPF1) score on a scale of 0 to 1, wherein 0 is indicative of a low rate of clearance below the predetermined threshold, and 1 is indicative of a high rate of clearance above the predetermined threshold;
   (c) calculating a poor prognosis factor 2 (PPF2) score on a scale of 0 to 2 with a second statistical model, based at least in part, on a presence of a genotype at a single nucleotide variant (SNV) comprising rs2097432 or a SNV in linkage disequilibrium therewith detected in the biological sample obtained from the subject, wherein 0 is indicative of the genotype that is homozygous non-risk, 1 is indicative of the genotype that is heterozygous risk at rs2097432, and 2 comprises a presence of two genetic loci of said genotype; and
   (d) calculating a combined PPF score comprising the PPF1 score and the PPF2 score to predict whether the immune-mediated inflammatory disease in the subject will positively respond to the treatment with the biologic drug.
83. The method of embodiment 82, further comprising:
   (e) calculating a poor prognosis factor 4 (PPF4) score on a scale of 0 to 1 with a third statistical model, based at least in part on patient wellbeing received from the subject, wherein a PPF4 score of 0 is indicative of an improvement of at least one symptom of the immune-mediated inflammatory disease, and a PPF4 score of 1 is indicative of no improvement in the at least one symptom, and wherein the combined PPF score calculated in (d) further comprises the PPF4 score.
84. The method of embodiment 82 or 83, further comprising: (f) calculating a poor prognosis factor 3 (PPF3) score on a scale of 0 to 2 with a fourth statistic model, wherein 0 is indicative of a standard response to the biologic drug, 1 is indicative e of a low response to the biologic drug, and 2 is indicative of very low response to the biologic drug.
85. The method of any one of embodiments 82-84, wherein the predetermined threshold is about 0.2 to 0.4 L/day.
86. The method of any one of embodiments 82-84, wherein the predetermined threshold is above 0.3 L/day.
87. The method of any one of embodiments 82-86, further comprising calculating a time to remission for the subject, wherein the time to remission is expressed as a percentage reduction in hazard remission.
88. The method of embodiment 87, wherein the percentage reduction in hazard remission is above 34% and the biologic drug comprises the biologic drug, such as for example, Infliximab.
89. The method of embodiment 87, further comprising administering to the subject a first dose of the biologic drug (e.g., 5 mg/Kg of Infliximab), provided the percentage reduction in hazard remission is above 78%.
90. The method of embodiment 87, further comprising administering to the subject a second dose of the biologic drug that is larger than the first dose of the biologic drug (e.g., 7.5 mg/Kg of Infliximab), provided the percentage reduction in hazard remission comprises from 56% to 78%.
91. The method of embodiment 87, further comprising administering to the subject a third dose of the biologic drug that is larger than the first dose and the second dose of the biologic drug (e.g., 10 mg/Kg of Infliximab), provided the percentage reduction in hazard remission comprises 34% to 56%.
92. The method of embodiment 87, further comprising administering to the subject a therapy other than the biologic drug, provided the percentage reduction in hazard remission is 34% or below.
93. The method of embodiment 92, wherein the therapy other than the biologic drug comprises a small molecule inhibitor of Janus Kinase (JAK) or a small molecule modulator of sphingosine 1-phosphate (S1P).
94. The method of embodiment 93, wherein the small molecule inhibitor of Janus Kinase (JAK) comprises Tofacitinib.
95. The method of any one of embodiments 82-94, wherein the first statistical model and the second statistical model comprises one or more algorithms comprising a Naïve Bayes classifier algorithm, a non-linear mixed effects model (NLME), or a Metropolis Hastings algorithm, or any combination thereof.
96. The method of any one of embodiments 82-95, wherein the biologic drug comprises Infliximab, adalimumab, etanercept, golimumab, or certolizumab.
97. The method of any one of embodiments 82-96, wherein the immune-mediated inflammatory disease comprises an inflammatory bowel disease (IBD), rheumatoid arthritis (RA), cytokine release syndrome, multiple sclerosis (MS), ankylosing spondylitis (AS), lupus, plaque psoriasis, atopic dermatitis, gout, migraine, cancer, or a neoplasm.
98. The method of embodiment 97, wherein the IBD comprises Crohn's disease.
99. The method of embodiment 97, wherein the IBD comprises Ulcerative Colitis.
100. The method of any one of embodiments 82-99, wherein the biological sample obtained from the subject is blood serum.
101. The method of any one of embodiments 82-100, wherein the genotype at the SNV comprising rs2097432 comprises a “G.”
102. The method of embodiment 101, wherein the genotype at the SNV comprising s2097432 is homozygous.
103. The method of any one of embodiments 82-102, wherein the linkage disequilibrium is determined with an r² of at least about 0.84.
104. The method of any one of embodiments 82-103, wherein the subject is age 18 or older.
105. The method of any one of embodiments 82-104, wherein the patient wellbeing data comprises a score on a disease activity index for the immune-mediated inflammatory disease.
106. The method of embodiment 105, wherein the disease activity score index comprises Crohn's Disease Activity Index (CDAI) or a Mayo Clinical Score.

### I. Definitions

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

As used in this specification and the appended claims, the singular forms “a,” “an,” and “the” include plural references unless the context clearly dictates otherwise. Any reference to “of” herein is intended to encompass “and/or” unless otherwise stated.

Reference throughout this specification to “some embodiments,” “further embodiments,” or “a particular embodiment,” means that a particular feature, structure, or characteristic described in connection with the embodiment is included in at least one embodiment. Thus, the appearances of the phrase “in some embodiments,” or “in further embodiments,” or “in a particular embodiment” in various places throughout this specification are not necessarily all referring to the same embodiment. Furthermore, the particular features, structures, or characteristics may be combined in any suitable manner in one or more embodiments.

The term “biologic drug profile” generally refers to a profile of a subject disclosed herein. The biologic drug profile can comprise a dose of a biologic drug and an inter-dose interval estimated to achieve a threshold biologic drug concentration in a subject sufficient to treat the disease. The threshold biologic drug concentration may be a pre-specified threshold concentration.

Non-limiting examples of “biological sample” include any material from which nucleic acids and/or proteins can be obtained. As non-limiting examples, this includes whole blood, peripheral blood, plasma, serum, saliva, mucus, urine, semen, lymph, fecal extract, cheek swab, cells or other bodily fluid or tissue, including but not limited to tissue obtained through surgical biopsy or surgical resection. In various embodiments, the sample comprises tissue from the large and/or small intestine. In various embodiments, the large intestine sample comprises the cecum, colon (the ascending colon, the transverse colon, the descending colon, and the sigmoid colon), rectum and/or the anal canal. In some embodiments, the small intestine sample comprises the duodenum, jejunum, and/or the ileum. Alternatively, a sample can be obtained through primary patient derived cell lines, or archived patient samples in the form of preserved samples, or fresh frozen samples.

The term, “clinical disease activity index (CDAI)” refers to the patient global assessment of disease activity for an immune-mediated inflammatory disease disclosed herein, which may change according to industry standards. In some embodiments, the clinical disease activity index is the Crohn's disease activity index (CDAI), which is disclosed in Best et al., Predicting the Crohn's disease activity index from the Harvey-Bradshaw Index. Inflammatory Bowel Diseases 2006, 12 (4): 304-10, which is hereby incorporate by reference in its entirety. In some embodiments, the CDAI is a CDAI for rheumatoid arthritis as described in Aletaha D, Nell V P, Stamm T, et. al. Acute phase reactants add little to composite disease activity indices for rheumatoid arthritis: validation of a clinical activity score. Arthritis Research & Therapy 2005, 7 (4): R796-806, which is hereby incorporated by reference in its entirety.

The terms “determining,” “measuring,” “evaluating,” “assessing,” “assaying,” and “analyzing” are often used interchangeably herein to refer to forms of measurement. The terms include determining if an element is present or not (for example, detection). These terms can include quantitative, qualitative or quantitative and qualitative determinations. Assessing can be relative or absolute. “Detecting the presence of” can include determining the amount of something present in addition to determining whether it is present or absent depending on the context.

The term “ex vivo” is used to describe an event that takes place outside of a subject's body. An ex vivo assay is not performed on a subject. Rather, it is performed upon a sample separate from a subject. An example of an ex vivo assay performed on a sample is an “in vitro” assay.

The term “indel” as disclosed herein, refers to an insertion, or a deletion, of a nucleobase within a polynucleotide sequence.

In some embodiments, the terms “individual” or “subject” are used interchangeably and refer to any animal, including, but not limited to, humans, non-human primates, rodents, and domestic and game animals, which is to be the recipient of a particular treatment. Primates include chimpanzees, cynomolgus monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. In various embodiments, a subject can be one who has been previously diagnosed with or identified as suffering from or having a condition in need of treatment. In certain embodiments, the subject is a human. In various other embodiments, the subject previously diagnosed with or identified as suffering from or having a condition may or may not have undergone treatment for a condition. In yet other embodiments, a subject can also be one who has not been previously diagnosed as having a condition (i.e., a subject who exhibits one or more risk factors for a condition). A “subject in need” of treatment for a particular condition can be a subject having that condition, diagnosed as having that condition, or at risk of developing that condition. In some embodiments, the subject is a “patient,” that has been diagnosed with a disease or condition described herein.

The term “inflammatory bowel disease” or “IBD” as used herein refers to gastrointestinal disorders of the gastrointestinal tract. Non-limiting examples of IBD include, Crohn's disease (CD), ulcerative colitis (UC), indeterminate colitis (IC), microscopic colitis, diversion colitis, Behcet's disease, and other inconclusive forms of IBD. In some instances, IBD comprises fibrosis, fibrostenosis, stricturing and/or penetrating disease, obstructive disease, or a disease that is refractory (e.g., mrUC, refractory CD), perianal CD, or other complicated forms of IBD.

The term “in vivo” is used to describe an event that takes place in a subject's body.

The term, “Linkage disequilibrium,” or “LD,” as used herein refers to the non-random association of alleles or indels in different gene loci in a given population. LD may be defined by a D′ value corresponding to the difference between an observed and expected allele or indel frequencies in the population (D=Pab−PaPb), which is scaled by the theoretical maximum value of D. LD may be defined by an r2 value corresponding to the difference between an observed and expected unit of risk frequencies in the population (D=Pab−PaPb), which is scaled by the individual frequencies of the different loci. In some embodiments, D′ comprises at least 0.20. In some embodiments, r² comprises at least 0.70.

The term “model” generally refers to a computer simulation used herein to predict an output based on certain inputs. In some embodiments, the computer simulation may employ statistical methods, numerical methods, machine learning methods, or any combination thereof. In some embodiments, the output is a recommended dose or inter-dose interval for a biologic drug, a likelihood of clinical remission, or both. In some embodiments, the input comprises one or more analytes disclosed herein (e.g., CRP, PR3, pANCA, antibodies against integrins, albumin, etc.), the body mass index (BMI) of the subject, the weight of the subject, information about the subject (e.g., disease severity, symptom severity and/or type, clinical remission status, age, gender, prior medial history, immune-compromising conditions, and the like).

The terms “non-response,” or “loss-of-response,” as used herein, refer to phenomena in which a subject or a patient does not respond to the induction of a treatment (e.g., anti-TNF therapy), or experiences a loss of response to the treatment after a successful induction of the therapy. The induction of the standard treatment may include 1, 2, 3, 4, or 5, doses of the therapy. A “successful induction” of the therapy may be an initial therapeutic response or benefit provided by the therapy. The loss of response may be characterized by a reappearance of symptoms consistent with a flare after a successful induction of the therapy.

The term “poor prognostic factor” or “PPF” generally refers to factors that can be used as predictors of achieving a predetermined output, such as for example, a pre-specified threshold concentration of the biologic drug or clinical remission of the disease in the subject. The PPF may comprise an estimated clearance rate of a biologic drug in a subject (e.g., PPF1), increased risk for developing autoantibodies against the biologic drug (e.g., PPF2), antigenic load (e.g., presence or amount of serological markers) (e.g., PPF3), presence of at least one symptom (e.g., PPF4) or any combination thereof.

The term “pre-specified threshold” generally refers to a target concentration level of an analyte, such as a drug. The term “pre-specified threshold” may be used interchangeably with “predetermined threshold” unless specified otherwise. A dose and an inter-dose interval of a drug administered to a subject may be adjusted based on a likelihood of achieving a pre-specified threshold of the drug in the patient as determined by a model disclosed herein.

The term “patient” or“subject” generally refers to an individual having a disease, such as, but not limited to, those disclosed herein. Subject specific data may be obtained from a patient or a subject, which may be used to establish a biologic drug profile for a patient or a subject.

The term “reference population” generally refers to a population of subjects. In some embodiments, the reference population is a population of subjects that have received a biologic drug for treatment of a disease or a condition disclosed herein. Generally, the subject is not part of the reference population. In some embodiments, the reference population comprises subjects that have received the same biologic drug as the subject. In some embodiments, the reference population may comprise subjects with the same disease as the subject. Data from the reference population may be used to develop and/or train a model of the present disclosure. In some embodiments, the model may be used to determine a treatment regimen for the subject, including, for example a dose or an inter-dose interval for a therapeutic agent disclosed herein.

The term “serological marker,” as used herein refers to a type of biomarker representing an antigenic response in a subject that may be detected in the serum of the subject. In some embodiments, a serological comprises an antibody against various fungal antigens. Non-limiting examples of a serological marker comprise anti-Saccharomyces cerevisiae antibody (ASCA), an anti-neutrophil cytoplasmic antibody (ANCA), E. coli outer membrane porin protein C (OmpC), anti-Malassezia restricta antibody, anti-Malassezia pachydermatis antibody, anti-Malassezia furfur antibody, anti-Malassezia globasa antibody, anti-Cladosporium albicans antibody, anti-laminaribiose antibody (ALCA), anti-chitobioside antibody (ACCA), anti-laminarin antibody, anti-chitin antibody, pANCA antibody, anti-I2 antibody, and anti-Cbir1 flagellin antibody.

The term, “single nucleotide variant” or SNV as disclosed herein, refers to a variation in a single nucleotide within a polynucleotide sequence. The term should not be interpreted as placing a restriction on a frequency of the SNV in a given population.

The terms “treat,” “treating,” and “treatment” as used herein refers to alleviating or abrogating a disorder, disease, or condition; or one or more of the symptoms associated with the disorder, disease, or condition; or alleviating or eradicating a cause of the disorder, disease, or condition itself. Desirable effects of treatment can include, but are not limited to, preventing occurrence or recurrence of disease, alleviation of symptoms, diminishing any direct or indirect pathological consequences of the disease, preventing metastasis, decreasing the rate of disease progression, amelioration or palliation of the disease state and remission or improved prognosis.

The section headings used herein are for organizational purposes only and are not to be construed as limiting the subject matter described.

### II. Examples

The following examples are included for illustrative purposes only and are not intended to limit the scope of the inventive concepts.

### Example 1. Building a Patient-Centric Precision Model for Predicting Time to Remission Following Induction of Infliximab

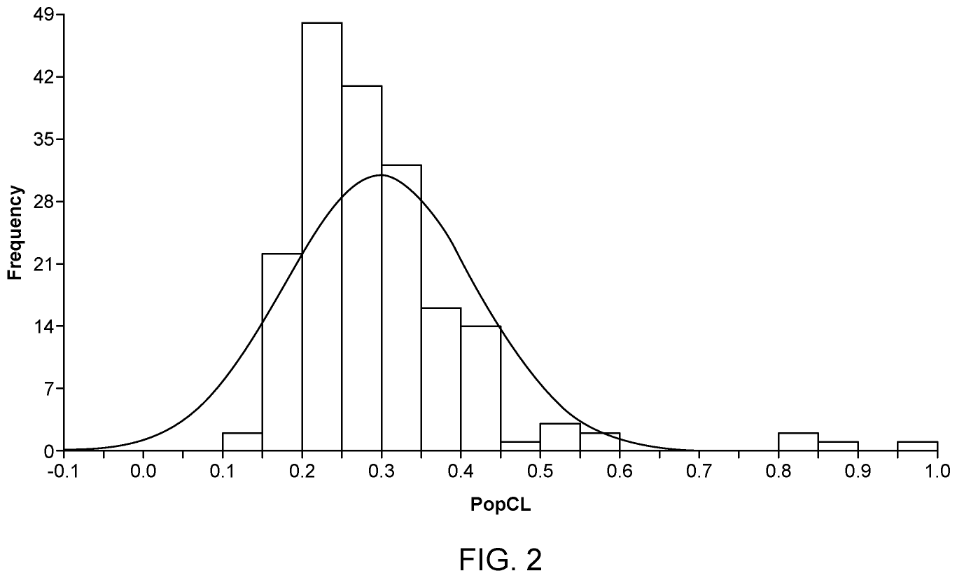

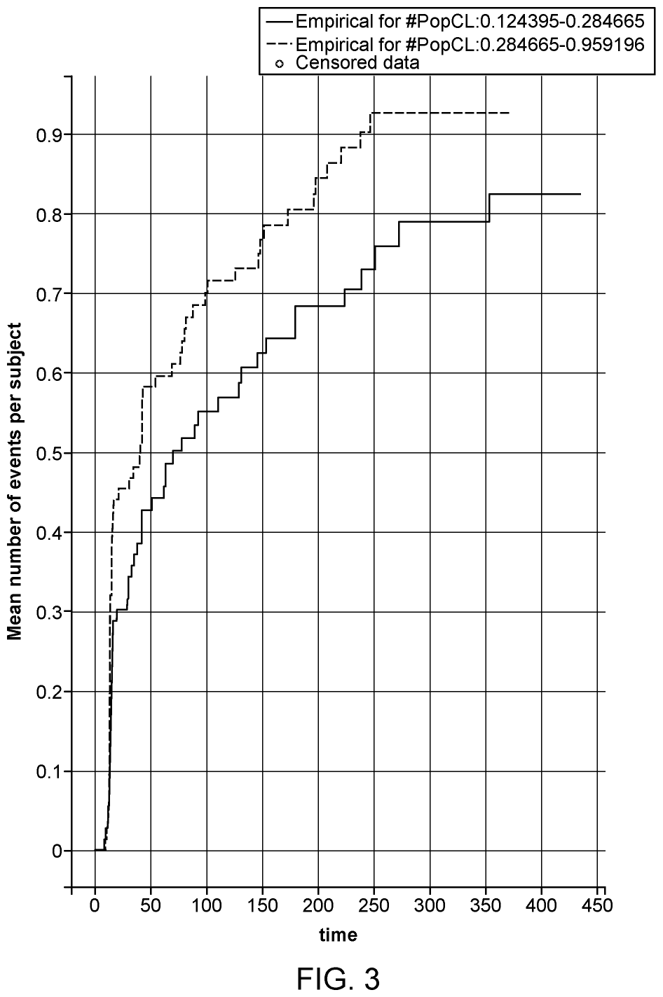

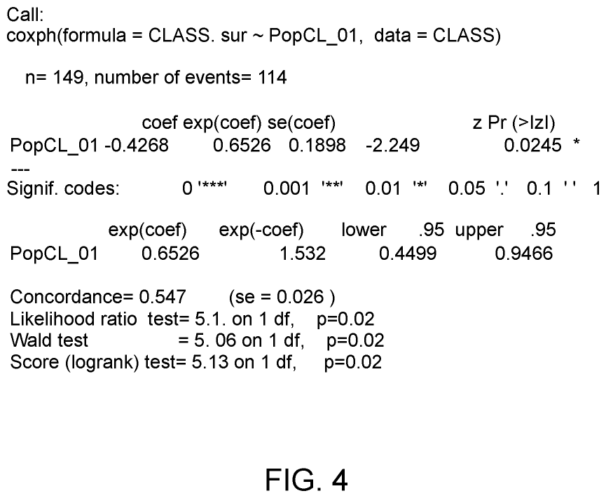

The structural model (also referred to herein as “Patient-Centric Precision Model”) for predicting time to disease remission was constructed from data from subjects suffering from inflammatory bowel disease. Biological samples were obtained from the subjects, analyzed for various levels of biomarkers using methods described in Example 4, which levels and other patient information was input into the structural model. Subjects were selected at baseline with active disease status in the presence of high C-reactive protein (CRP) (>3 mg/L; N=119 subjects). The pharmacokinetic systems in subjects were established using albumin and weight as covariates, which was indicative of their propensity to clear a drug from their system. The clearance was collected at baseline at the point of the encounter (e.g., when the patient visits the clinician's office for the first time), which is shown in FIG. 2 illustrating baseline clearance in subjects. As shown in FIG. 2, some patients may present with intrinsically high clearance (above 0.3 L/day) and were likely to present with reduction in the hazard function (remission). Further, FIG. 3 illustrates the time to remission related to this baseline clearance. The time of remission was defined by the absence of symptoms and CRP levels below normal range. In this example, 3 mg/L of Infliximab was used as a cutoff. As shown, clearance above 0.28 L/day reduced the hazard of remission by 40% (HR=0.6 CI95%: 0.4-0.9). FIG. 4 shows a computer interface used for calculating baseline clearance in subjects. The clearance was calculated using the following formula: ln(Cl)=ln(0.294)−1.17*ln(Albumin (g/dL)/4)+0.614*ln(Weight (kg)/70).

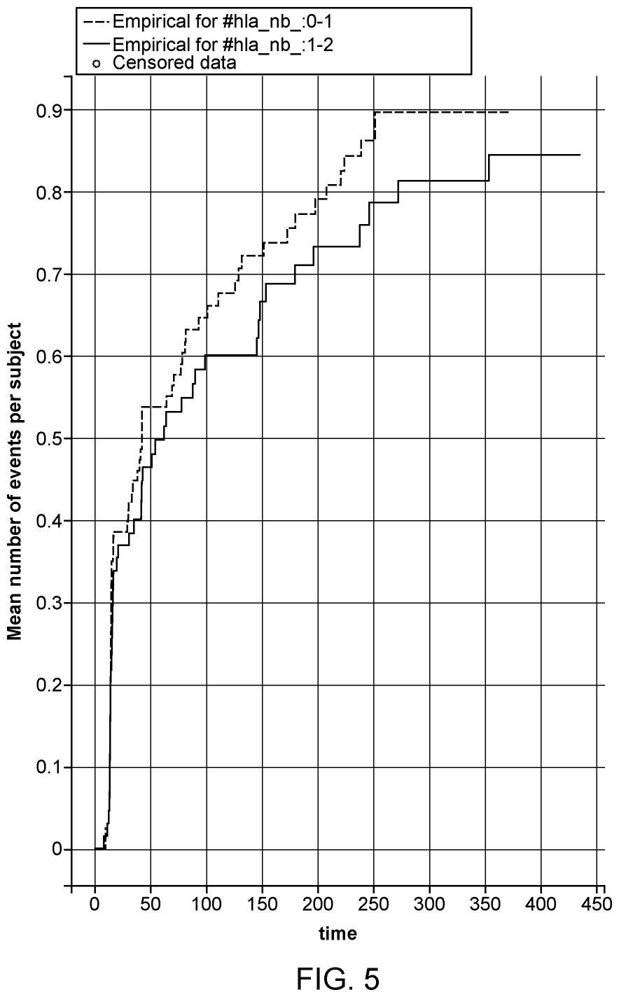

Genotypes were obtained from the subject from a genotyping device using the standard operating procedure provided in Example 4. The genetic system was established based on the number of alleles (n=0 (AA), n=1 (AG), n=2 (GG)) of HLA DQA1*05 detected in a biological sampled obtained from the subject that was collected at baseline at the point of encounter. FIG. 5 shows the time to remission based on the number of alleles of HLA DQA1*05 in a subject. As shown in FIG. 5, the presence of both alleles for HLA DQA1*05 reduced the hazard of remission. The pharmacokinetic system and the genetic system were combined into a structure model and scores were assigned, as shown in Table 1. Table 1 also shows the reduction in hazard remission and interventions depending on the aggregated score. The score calculated is referred to herein as “poor prognosis factor (PPF) score.

TABLE 1

| | | | Reduction in | | |
|---|---|---|---|---|---|
| | | HLA | Hazard of | | |
| PPF | Clearance | DQA1*05 | Remission | Intervention | |
| 0 | 0 | 0 | 100% | 5 | mg/Kg |
| 1 | 0 or 1 | 1 or 0 | 78% | 7.5 | mg/Kg |
| 2 | 1 | 1 | 56% | 10 | mg/Kg |

| | | | | |
|---|---|---|---|---|
| 3 | 1 | 2 | 34% | small molecule inhibitor |

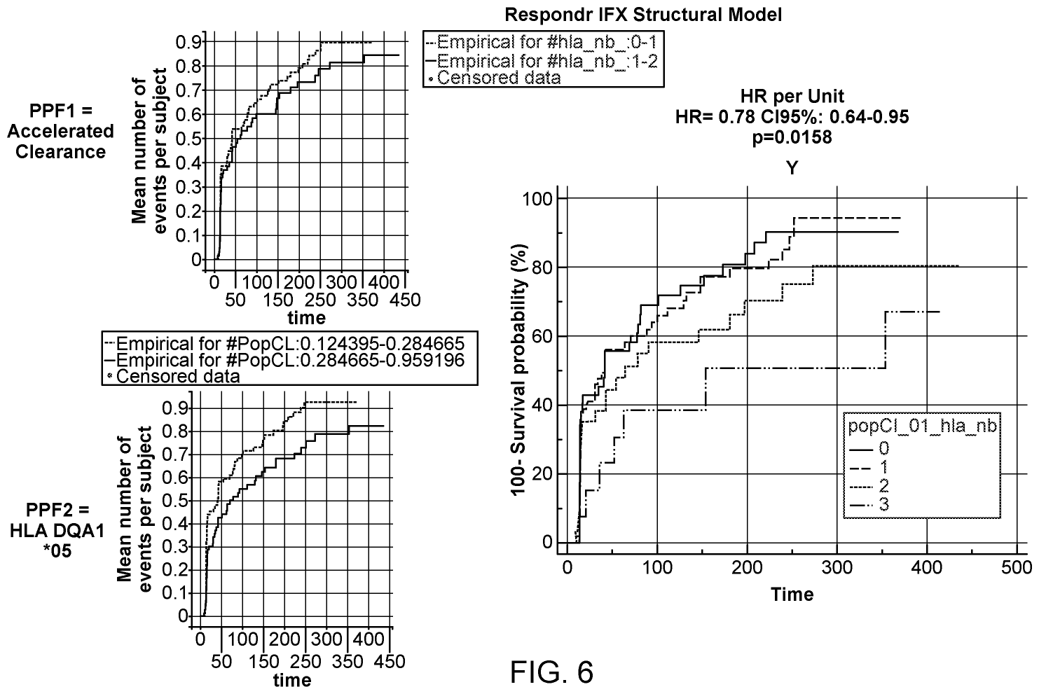

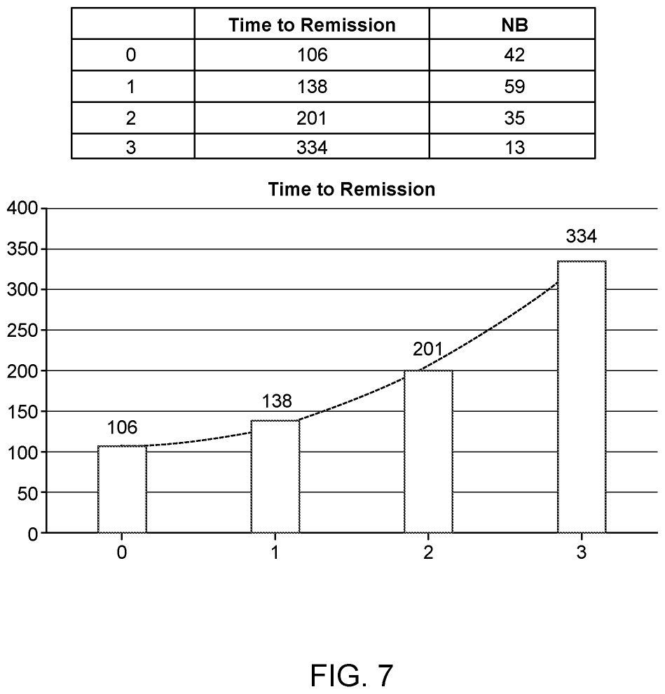

Further, FIG. 6 illustrates a time to remission of the combined pharmacokinetic and genetic system, which shows how the time to remission vary depending on the calculated PPF scores. Table 2 shows the time to remission function and the calculated time to remissions in IBD subjects. The time to remission function was calculated by log(Te)=log(Te_pop)+beta_Te_popCl_01 hla_nb*popCl_01 hla_nb+eta_Te, where Te_pop corresponds to the time to remission achievement at the population level, beta_Te_popCl_01_hla_nb corresponds to the covariate estimates associated with the aggregated HLA and clearance estimate, and eta_Te corresponds to the random effects associated with the population estimate. The times to remissions are shown in FIG. 7, which shows that patients with higher PPF scores experienced longer times (in days) to remission, whereas patients with lower PPF scores experiences shorter times to remission. In this example the time to event was calculated as follows: log(Te)=log(64.07)+0.39*Cl_01_hla_nb, using the estimates from the time to remission for the individual is calculated.

The time to remission may be used to by a clinician to optimize a therapeutic regiment for the subject. For example, if the subject has a combined PPF score of 3 indicative of 334 days until disease remission on an anti-TNF therapy, a clinician may prescribe a small molecule inhibitor to the subject instead, such as a small molecule inhibitor against Janus kinase (JAK) or sphingosine 1-phosphate (S1P) receptor. By contrast, a subject predicted to experience disease remission in 106 days (corresponding to a combined PPF score of 0) may be better suited for an anti-TNF therapy, such as Adalimumab or Infliximab.

TABLE 2

STOCH. APPROX.

| | | |
|---|---|---|
| VALUE | S.E. | R.S.E.(%) |

Fixed Effects

| | | | |
|---|---|---|---|
| Te_pop | 64.07 | 12.49 | 19.5 |
| beta_Te_popCl_01_hla_nb | 0.39 | 0.14 | 35.4 |
| h_cov_pop | 1 | | |

Standard Deviation of the Random Effects

| | | | |
|---|---|---|---|
| omega_Te | 0.91 | 0.15 | 16.5 |

“Te_pop” refers to the population estimate of the event variable;
“beta_Te_popCl_01_hla_nb” refers to the covariate estimate associated with the PPF combining Clearance and the number of HLA alleles carried by the individual
“omega_Te” refers to the random effect estimate of the model

### Example 2. Building a Patient-Centric Precision Model for Predicting a Subject's Time to Remission Following Induction of an Anti-TNF Therapy

The structure model (also referred to herein as “Patient-Centric Precision Model”) was constructed from data from subjects suffering from inflammatory bowel disease. The subjects included those who had not received an anti-TNF therapy and those who had received an anti-TNF therapy. In this example, the anti-TNF therapy that the subjects received was Infliximab. The pharmacokinetic systems was established using the weight and CRP levels of the subjects as covariates, as described in Example 1. The genetic system was established based the on the number of alleles of HLA DQA1*05, as described in Example 1. The poor prognosis factors for these subjects who had not received an anti-TNF therapy were referred to as those whose performances were measured in a proactive setting. The poor prognosis factors for these subjects who had received an anti-TNF therapy were referred to as those whose performances were measured in a reactive setting. The subjects that were selected for this time-to-event (TTE) analysis presented with active disease status, which included the presence of inflammation with CRP above 3 mg/L and/or Crohn's Disease Activity Index (CDAI)>150 points for Crohn's disease and/or Partial Mayo≥2 points for Ulcerative Colitis. All patients were followed longitudinally for their time to achieve adequate disease control (normal CRP in the absence of symptoms).

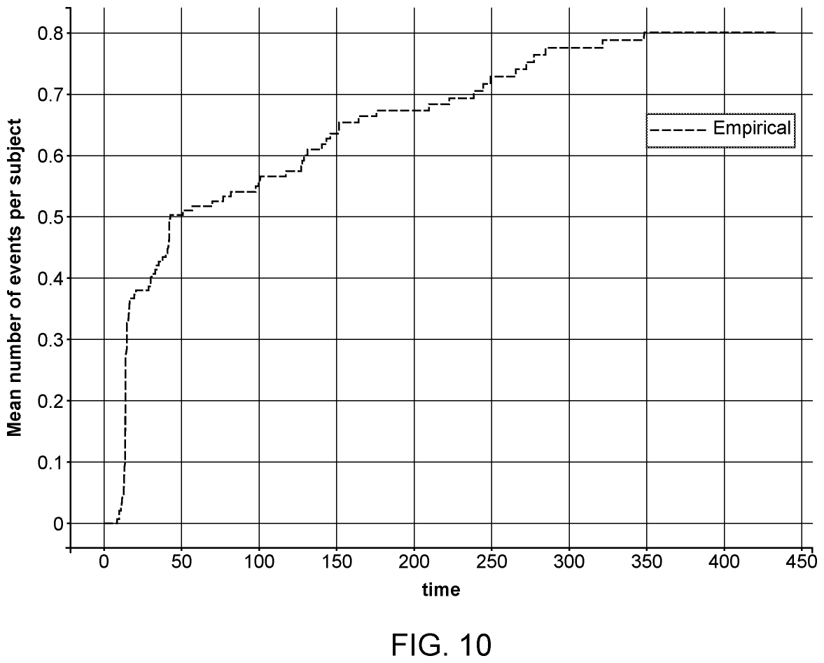

The time to remission estimates during treatment were measured using the function: log(Te)=log (Te_pop)+eta_Te, which is also illustrated in FIG. 10. Table 3 further shows the time to remission function and the calculated time to remissions in subjects. The estimates of the time to remission were calculated in the absence of symptoms and normal CRP. On a population basis, time to remission was achieved at 115 days, or approximately 16 weeks. As shown here, subjects with a presence of a risk genotype at the HLA DQA1*05 locus and estimated clearance of the anti-TNF therapy above the predetermined cutoff of 0.294 L/d are predictors of a longer time to remission, relative to subjects that do not carry a risk genotype at the HLA DQA1*05 locus and clearance is estimated to be below 0.294 L/d. These findings are independent of CRP levels or presence of symptoms.

TABLE 3

STOCH. APPROX.

| | | |
|---|---|---|
| VALUE | S.E. | R.S.E.(%) |

Fixed Effects

| | | | |
|---|---|---|---|
| Te_pop | 1115.76 | 16.9 | 14.6 |

Standard Deviation of the Random Effects

| | | | |
|---|---|---|---|
| omega_Te | 1.24 | 0.15 | 11.9 |

“Te_pop” refers to population estimate of the event and “omega_Te” refers to random effect estimate.

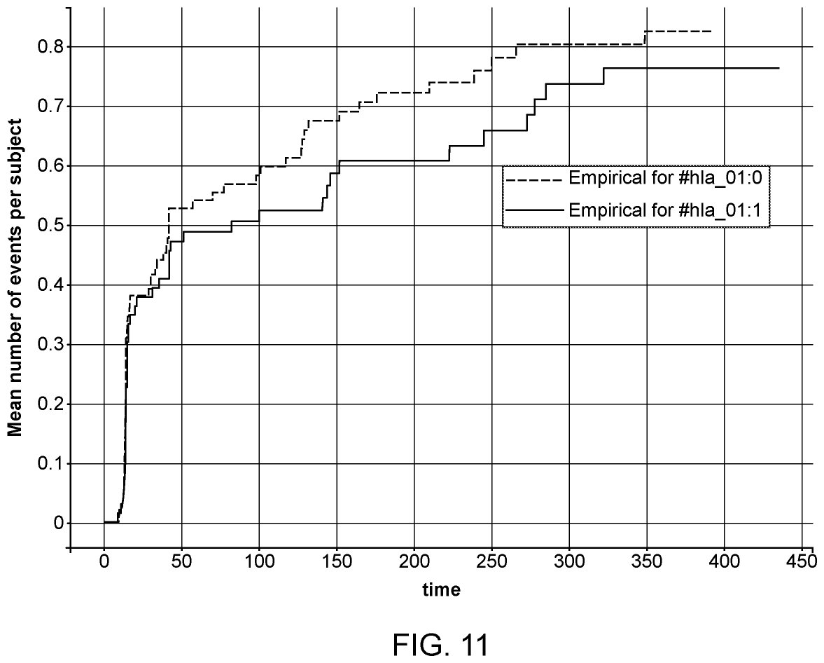

FIG. 11 shows the time to remission based on the number of alleles of HLA DQA1*05 in a subject. As shown in FIG. 11, the presence of both alleles for HLA DQA1*05 did not significantly reduce the hazard of remission. Table 4 shows the time to remission function and the calculated time to remissions in subjects. The time to remission function may be calculated by log(Te)=log(Te_pop)+beta_Te_hla_01_1*[hla_01=1]+eta_Te, where beta_Te_hla_01_1 corresponds to covariate estimates (e.g., 0.38 in Table 4) associated with the HLA estimate. On a population basis time to remission occurred at 99 days in the absence of HLA carrier status (AG, GG) as compared to 144 days in its presence. As shown here, presence of a risk genotype at the HLA DQA1*05 locus is a predictor or a longer time to remission as determined using the patient-centric precision model.

TABLE 4

STOCH. APPROX.

| | | |
|---|---|---|
| VALUE | S.E. | R.S.E.(%) |

Fixed Effects

| | | | |
|---|---|---|---|
| Te_pop | 99.09 | 19.21 | 19.4 |
| beta_Te_hla_01_1 | 0.38 | 0.29 | 76.3 |

Standard Deviation of the Random Effects

| | | | |
|---|---|---|---|
| omega_Te | 1.23 | 0.15 | 12.0 |

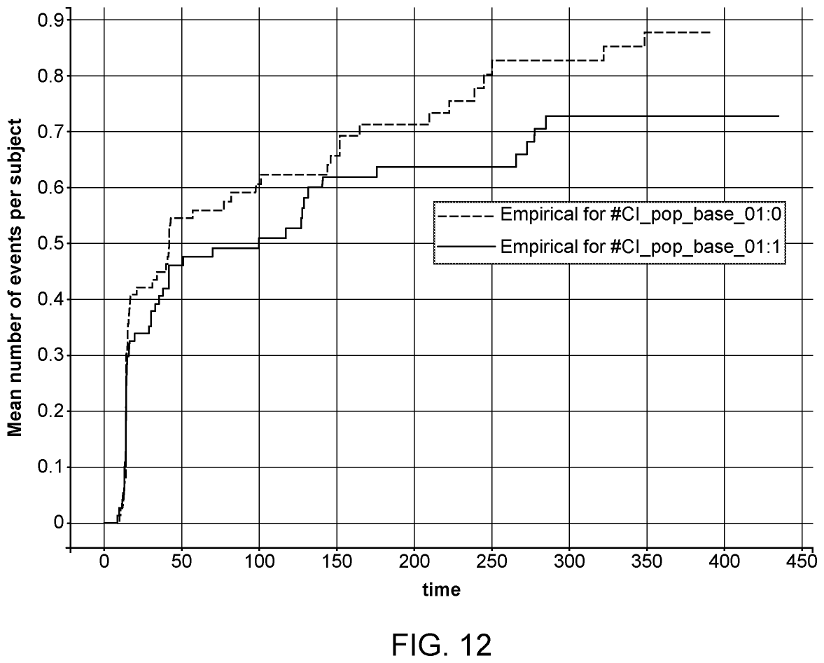

FIG. 12 illustrates the time to remission related to the baseline clearance of the pharmacokinetic system. As shown in FIG. 12 the clearance estimates did not significantly reduce the hazard of Remission (e.g., impact the time to remission). Table 5 shows the time to remission function and the calculated time to remissions in subjects. The time to remission function may be calculated by log(Te)=log(Te_pop)+beta_Te_Cl_pop_base_01_1*[Cl_pop_base_01=1]+eta_Te, where beta_Te_Cl_pop_base_01_1 corresponds to covariate estimates (e.g., 0.40 in Table 5) associated with the clearance estimate. On a population basis time to remission occurred at 96 days in the absence of accelerate clearance as compared to 143 days in its presence.

TABLE 5

| | STOCH. APPROX. | |
|---|---|---|
| VALUE | S.E. | R.S.E.(%) |

Fixed Effects

| | | | |
|---|---|---|---|
| Te_pop | 96.01 | 19.51 | 20.3 |
| beta_Te_Cl_pop_base_01_1 | 0.40 | 0.29 | 73.6 |

Standard Deviation of the Random Effects

| | | | |
|---|---|---|---|
| omega_Te | 1.26 | 0.15 | 11.8 |

Based on the data in FIG. 11 and FIG. 12, the subjects in a proactive setting (e.g., during treatment with the anti-TNF therapy) did not independently associate the pharmacokinetic and genetic system described herein with a significantly reduced hazard of remission.

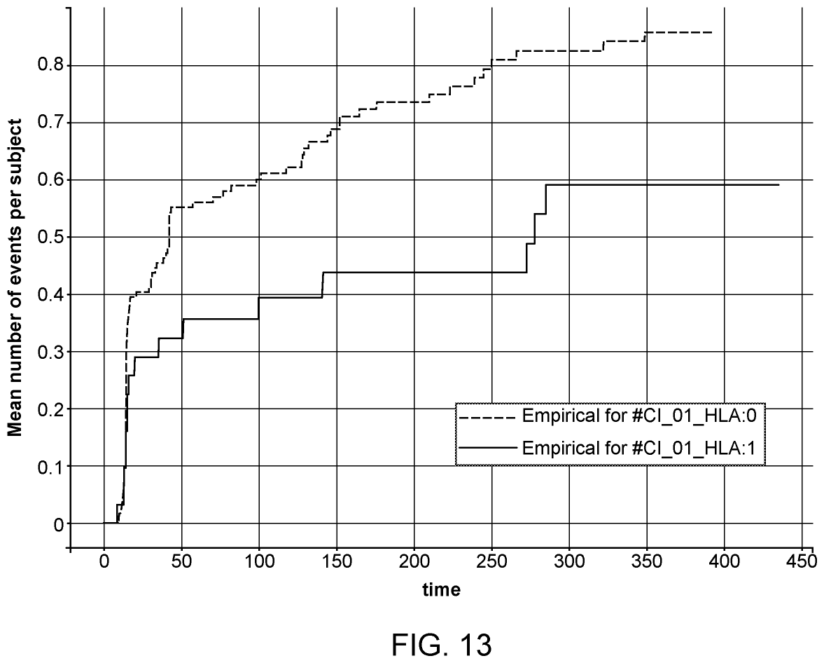

Further, FIG. 13 illustrates a time to remission of the combined pharmacokinetic and genetic system, which identified patients with a longer time-to-event (“TTE”). In this example TTE refers to time to remission. The function and parameters for FIG. 13 are shown in Table 6 and was calculated by log(Te)=log(Te_pop)+beta_Te_popCl_01_hla_1*[Cl_01 hla_1]+eta_Te, where beta_Te_popCl_01_hla_1 corresponds to the covariate estimates associated with the aggregated HLA and clearance estimate. FIG. 13 shows how the time to remission vary depending on the calculated PPF scores. The time to remission occurred at 94 days in the absence of both PPF as compared to 300 days in their presence. Thus, the combined PPF score of the pharmacokinetic and genetic systems (PPF 1 and PPF2) can be used to predict time to remission for a subject. Generally, an absence of the risk genotype (PPF2=0) and clearance below 0.294 L/d (PPF=0) is predictive of a shorter time to remission than a presence of the risk genotype (PPF2=1 or 2) and clearance above 0.294 L/d (PPF=1).

TABLE 6

STOCH. APPROX.

| | | |
|---|---|---|
| VALUE | S.E. | R.S.E.(%) |

Fixed Effects

| | | | |
|---|---|---|---|
| Te_pop | 94.15 | 15.08 | 16.0 |
| beta_Te_Cl_01_hla_1 | 1.14 | 0.38 | 33.5 |

Standard Deviation of the Random Effects

| | | | |
|---|---|---|---|
| omega_Te | 1.21 | 0.16 | 13.1 |

“Te_pop” refers to population estimate;
“beta_Te_Cl_01_hla_1” refers to the combination of Clearance (PPF1) and HLA DQA1*05 (PPF2)
“omega_Te” refers to random effect estimate.

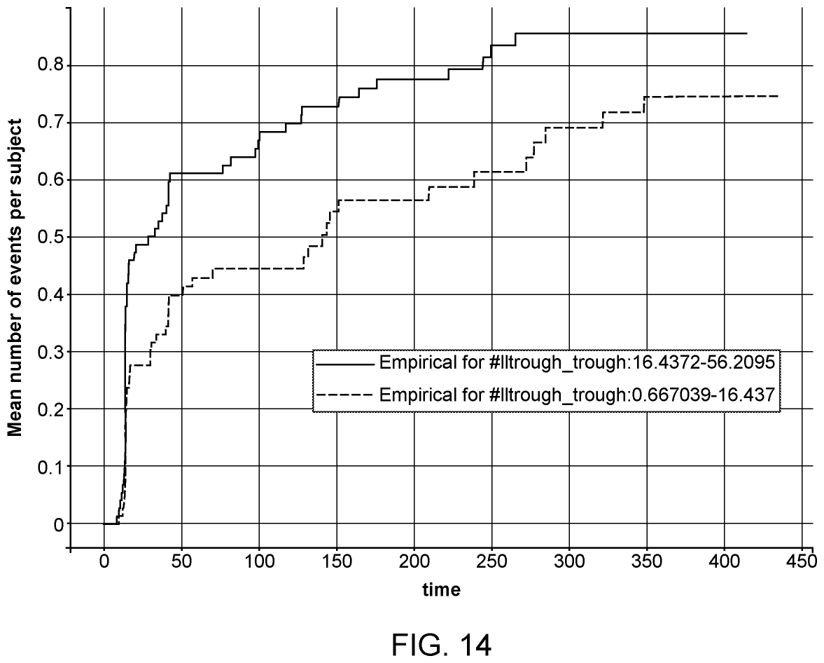

FIG. 14 illustrates a time to remission based on proactive maintenance of Infliximab (IFX) levels. FIG. 14 shows how pre-infusion IFX levels below 15 mg/L elongated the time to remission in subjects. The function and parameters for FIG. 14 are shown in Table 7 and was calculated by log(Te)=log(Te_pop)+beta_Te_IITrough_lt_15+eta_Te, where beta_Te_IItrough_lt_15_1 corresponds to the covariate estimates associated with pre-infusion IFX levels below 15 mg/L. On a population basis TTE occurred at 81 days in the presence of IFX levels>15 mg/L, whereas TTE occurred at 189 days when IFX<15 mg/L. Thus, the lower the level of a biologic drug (e.g., IFX), the lower the number of days to disease remission.

TABLE 7

STOCH. APPROX.

| | | |
|---|---|---|
| VALUE | S.E. | R.S.E.(%) |

Fixed Effects

| | | | |
|---|---|---|---|
| Te_pop | 81.83 | 15.16 | 18.5 |
| beta_Te_IItrough_lt_15_1 | 0.85 | 0.3 | 34.9 |

Standard Deviation of the Random Effects

| | | | |
|---|---|---|---|
| omega_Te | 1.2 | 0.16 | 13.2 |

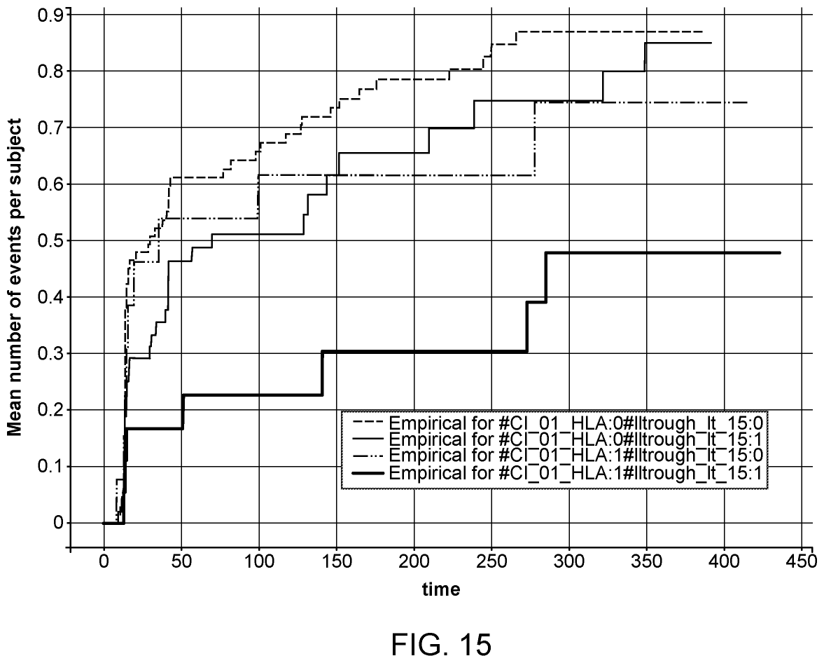

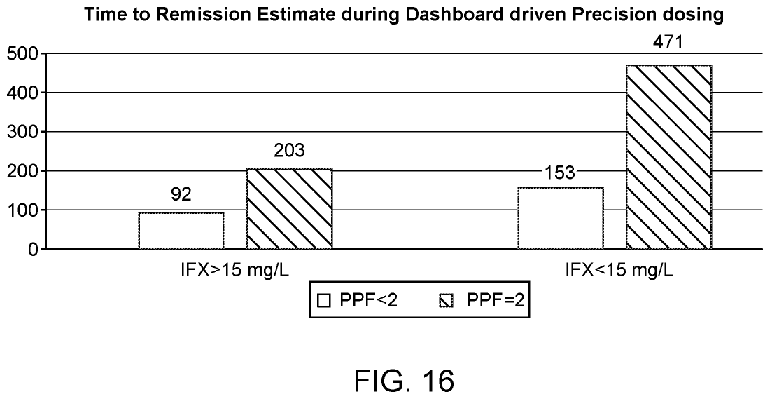

A multivariate analysis of the time to remission with combined pharmacokinetic and genetic system, as well as infusion IFX levels during treatment were performed and is shown in FIG. 15. The function and parameters for FIG. 15 are shown in Table 8. FIG. 15 shows that the time to remission is reduced in subjects with low IFX levels and when the pharmacokinetic and genetic system are combined to achieve a score of 3 consisting of the presence of both risk genotypes (PPF2 score=2) and pharmacokinetic system (PPF1 score=1). This is further illustrated in FIG. 16, in which the time to remission estimate is higher in subjects with low IFX levels and a combined PPF score from the pharmacokinetic and genetic system of 2, as discussed in Example 1.

TABLE 8

STOCH. APPROX.

| | | |
|---|---|---|
| VALUE | S.E. | R.S.E.(%) |

Fixed Effects

| | | | |
|---|---|---|---|
| Te_pop | 71.14 | 13.38 | 18.8 |
| beta_Te_popCl_01_hla_nb | 1.03 | 0.38 | 37.3 |
| beta_Te_IItrough_lt_15_1 | 0.67 | 0.3 | 44.3 |

Standard Deviation of the Random Effects

| | | | |
|---|---|---|---|
| omega_Te | 1.17 | 0.15 | 13.2 |

### Example 3. Poor Prognosis Pharmacokinetic Factors During Infliximab Induction Impact Time to Remission in Inflammatory Bowel Disease Patients

In this example, the PPF1 was calculated based on estimated drug clearance and low end cycle IFX levels determined during induction of IFX to assess the likelihood of inadequate disease control during maintenance therapy of Inflammatory Bowel Disease (IBD). The population of patients with IBD comprised patients with Crohn's disease and Ulcerative Colitis. The first cohort comprised patients with IBD who were receiving IFX therapy. According to the protocol for the IFX therapy, patients were administered three induction doses of 5 mg/Kg, at week 0, 2, and 6, followed by every 8 weeks maintenance at the same dose. The second cohort comprised patients with IBD who were proactively controlled for optimal exposure by Bayesian dashboard and precision dosing. Disease activity was assessed using CDAI and Harvey-Bradshaw Index (HBI). Inflammation was assessed using CRP. CRP-based clinical remission status was classified as CRP levels below 3 mg/L in the absence of symptoms (CDAI<150 points, HBI<5 points and pMayoK<2 points, as appropriate). Analysis included semiparametric and parametric time to event analysis. The estimated Poor Prognostic “PF” factors at weeks 2, 6, and 14 were estimated using the Bayesian data assimilation method of Weight, Albumin, IFX drug level determined using homogenous mobility shift assay from serum collected.

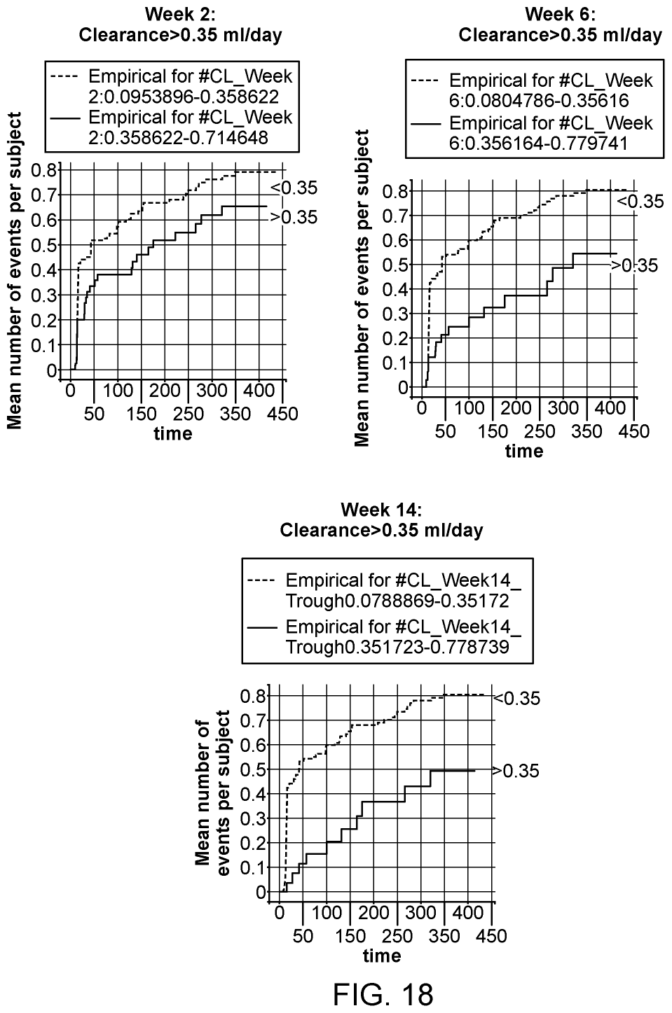

A time to remission analysis with induction estimates at weeks 2, 6, and 14 are shown in FIG. 18 for clearance levels>0.35 milliliters per day (ml/day). A mean number of events of as measured by the disease activity and inflammation, as described above, per subject were measured across several weeks. As shown, the mean number of events per subject remained elevated through week 2, 6, and 14 for patient with clearance<0.35 ml/day, while the mean number of events per subject decreased over week 2, 6, and 14 for patients with clearance>0.35 ml/day.

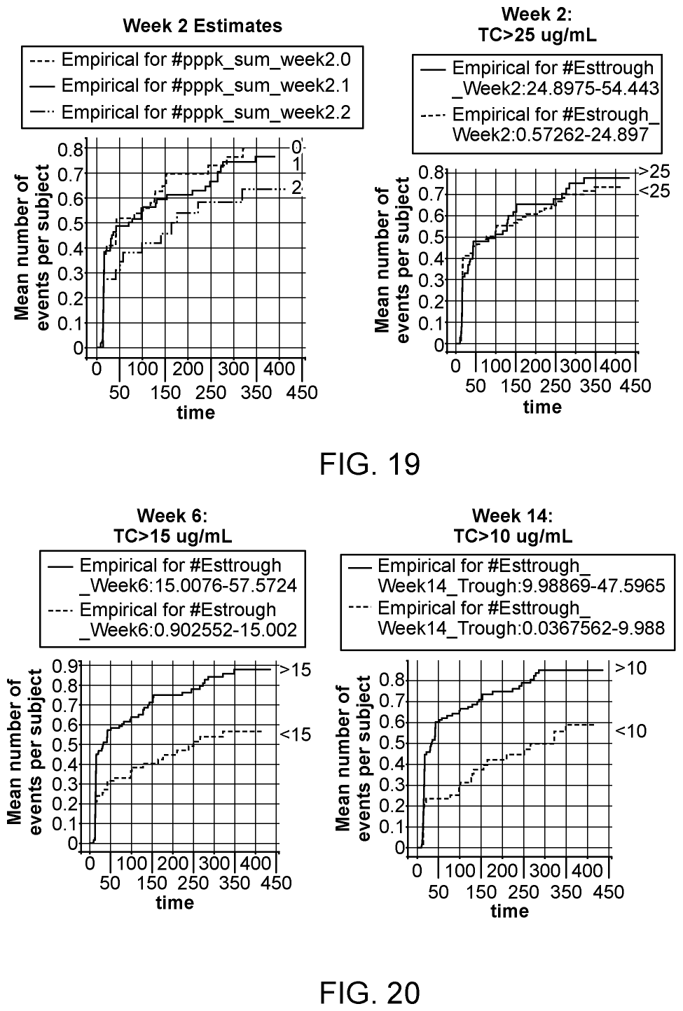

A time to remission analysis with induction estimated at week 2, 6, and 14, are also shown in FIG. 19 for a PF factor comprising varying trough concentration (“TC”) across the weeks. At week 2, patients with TC>25 μg/ml and TC<25 μg/ml have relatively similar mean numbers of events. At week 6 and 14, patients with TC<15 μg/ml and TC<10 μg/ml, respectively, have lower mean numbers of events compared to those with TC>15 μg/ml and TC>10 μg/ml, respectively.

The trough concentration (“TC”)(FIG. 19) were combined with accelerated clearance (FIG. 18) to produce a poor prognostic factor (PPF) score (ranging from 0 to 2), which is shown in FIG. 20. The parameters for the functions shown in FIG. 20 are shown in Tables 9, 10, and 11, for week 2, 6, and 14, respectively. The functions were calculated according to formulas in Example 2, where beta_Te_pppk_sum_week2_1 and beta_Te_pppk_sum_week2_2 correspond to the covariate estimates associated with the clearance and trough levels at week 2 for patients in the first cohort and second cohort, respectively.

TABLE 9

| | STOCH. APPROX. | |
|---|---|---|
| VALUE | S.E. | R.S.E.(%) |

Fixed Effects

| | | | |
|---|---|---|---|
| Te_pop | 117.91 | 31.44 | 26.7 |
| beta_Te_pppk_sum_week2_1 | 0.15 | 0.35 | 235 |
| beta_Te_pppk_sum_week2_2 | 0.81 | 0.46 | 56.1 |

Standard Deviation of the Random Effects

| | | | |
|---|---|---|---|
| omega_Te | 1.45 | 0.15 | 10.4 |

TABLE 10

| | STOCH. APPROX. | |
|---|---|---|
| VALUE | S.E. | R.S.E.(%) |

Fixed Effects

| | | | |
|---|---|---|---|
| Te_pop | 101.61 | 27.45 | 27.0 |
| beta_Te_pppk_sum_week6_1 | 0.13 | 0.34 | 252 |
| beta_Te_pppk_sum_week6_2 | 1.44 | 0.44 | 30.3 |

Standard Deviation of the Random Effects

| | | | |
|---|---|---|---|
| omega_Te | 1.36 | 0.14 | 10.5 |

TABLE 11

| | STOCH. APPROX. | |
|---|---|---|
| VALUE | S.E. | R.S.E.(%) |

Fixed Effects

| | | | |
|---|---|---|---|
| Te_pop | 73.67 | 12.97 | 17.6 |
| beta_Te_pppk_sum_week14_1 | 1.39 | 0.32 | 22.8 |
| beta_Te_pppk_sum_week14_2 | 2.44 | 0.53 | 21.9 |

Standard Deviation of the Random Effects

| | | | |
|---|---|---|---|
| omega_Te | 1.22 | 0.14 | 11.3 |

FIG. 20 shows the time to remission analysis with induction estimates based on the combined poor prognosis pharmacokinetic factors for week 2, 6, and 14. A PPF score of 0 indicated patients that did not have a low trough or high clearance. A PPF score of 1 indicated patients with either a low trough or high clearance. A PPF score of 2 indicated patients with low trough and high clearance. FIG. 20 illustrates how the time to remission varies depending on the calculated PPF scores. At week 2, the mean number of events per subject remain relatively high for all PPF scores. However, at week 6, the levels decreased for a PPF score of 2. Furthermore, at week 14, while the levels for a PPF score of 0 remained unchanged, the levels decreased for a PPF score of 1 and decreased further for a PPF score of 2.

### Example 4. Analyzing Biological Sample

For the above examples, biological samples were obtained from subjects in each study, and analyzed for levels of various biomarkers that were used as input into the structural model.

### Obtaining a Sample

Individual serum samples from healthy controls were obtained from blood bank donors (Golden West Biologics, Temecula, CA). Sera from patients with rheumatoid arthritis (RA), PS and inflammatory bowel disease (IBD) treated with adalimumab (ADA) every 14 days were drawn according to a protocol approved by an Institutional Review Board/Ethics Committee. Unless otherwise noted, all reagents and chemicals were obtained from Thermo Fisher Scientific (Waltham, MA) or Sigma Aldrich Corporation (St. Louis, MO). ADA and anti-ADA antibody levels were determined using homogenous mobility shift assay.

### Measuring PR3

Specimens analyzed for proteinase 3 (PR3) were serum at a minimum of 0.3 mL in a 4 mL cryovial, stored at 2-8° C. Specimens were analyzed for levels of PR3<20 CU=Negative; PR3≥20 CU=Positive. PR3 levels were analyzed using QUANTA Flash CIA procedure according to manufacturer's instructions. Chemiluminescence was detected using the BIO-FLASH Chemiluminescent Analyzer. Quantitative and qualitative analysis was performed.

Measuring pANCA

pANCA was measured using indirect immunofluorescent technique (IFA) Neutrophil-specific Nuclear Antibody (NSNA) in patient serum samples. Specimen is serum separated from cells as soon as possible, which were shipped overnight at room temperature, and stored up to seven days at 2-8° C. pANCA was measured using the following protocol:
1. Dilute the PBS II Concentrate from one of the Inova IFA kits 1:40 by adding the contents of the PBS II Concentrate Bottle to 975 mL of Di-Water. Alternatively, a previous dilution solution can be used as long as it is within seven days from dilution. Mix thoroughly before use.
2. Load the patient samples, deep wells, and PBS II buffer (from step 5.1.2) onto the Tecan EVO deck.
3. Dilute patients and controls on the Tecan
4. Recap samples for storage.
5. Vortex the dilution Deep Wells using an orbital mixer.
6. Add one drop (20-25 uL) of each undiluted Kit Positive and Negative controls to each slide type in wells 1-3.
7. Add 25 μL of each diluted patient sample to the appropriate wells on each slide type.
8. Place slides on a damp paper towel to keep them moist and incubate for 30±5 min.
9. Wash the slides in IBD Wash Buffer on a rocking shaker platform for three to five minutes.
10. Repeat wash one additional time.
11. Tap off the excess wash buffer and coverslip each slide with approximately 10 μL of the Kit Mounting Medium per reaction field. Store mounted slides protected from light and refrigerate (2-8° C.) for up to two (2) weeks.
12. Read using NOVA View Automated Microscope.

### Measuring Genotypes

The HLA DQA1*05A>G SNP test (chr6:32,622,994 A>G) was performed using qualitative PCR based genotyping assay that uses Taqman based fluorescence-quencher technology to detect the SNP. The assay is comprised of 2×PCR primers and 2×dual-labeled probes. The 2 PCR primers (FWD/REV) amplify the target sequence. The 2 dual-labeled probes each have different binding specificities, one for the Wild Type (reference) sequence and the other for the variant sequence. Each probe oligonucleotide sequence is dual-labeled, with a 5′-fluorophore and a 3′-quencher. The proximity of the quencher to the fluorophore inhibits the fluorescent signal while both are attached to the probe. Each PCR cycle causes the DNA polymerase enzyme to cleave the fluorophore from the probe bound to its target sequence. This leads to an increase on fluorescence. This increase of detached fluorophore doubles with each PCR cycle. This increase of fluorescence is detected by the Applied Biosystems 7500 real-time instrument attributed to the detection of the target sequence based on the probe specificity.

The TaqMan® based genotyping assays are qualitative tests. Data is collected from endpoint fluorescent reads to detect two different fluorophores (FAM & HEX). The resulting signals cluster into 3 distinct genotypes homozygous non-risk (AA), homozygous risk (GG) and heterozygous (AG).

Lysates and/or gDNA samples from whole blood are prepared. PCR master mix was prepared and the plate was loaded with master mix, samples and controls. Baseline fluorescent read of the plat on Applied Biosystems™ (ABI) 7500 Real-Time PCR System was performed. Amplification cycles were run on ABI Veriti™ thermocycler. A final fluorescence readout is observed using the ABI 7500.

### Measuring CRP

Quantitative C-Reactive protein (CRP) was measured in serum samples with volume of about 150 μl using the Beckman Coulter® IMMAGE® 800 system according to manufacturer's instructions, wherein the instrument measures the rate of increase in light-scatter resulting from an immunoprecipitation reaction occurring between human CRP antigen in a sample and the antibody to CRP in antiserum.

### Measuring Albumin

Human Albumin (ALB) was measured in serum samples with volume of about 150 μl using the Beckman Coulter® IMMAGE® 800 system according to manufacturer's instructions, wherein the instrument measures the rate of increase in light-scatter resulting from an immunoprecipitation reaction occurring between human ALB in a sample and the antibody to ALB in antiserum.

### Example 6. Patient Wellbeing Information

Patient wellbeing information used in the PPF4 model of the structural model is obtained from the patient and used to predict time to disease remission in an IBD patient. Such information includes the subject's weight, disease severity (e.g., remission, recurrence, or type, frequency, and/or severity of symptoms), disease activity using a disease activity index such as those described herein, or overall wellness using a standard, such as, e.g., SF-36 or EQ5D among others disclosed herein. The wellbeing information is collected from the subject remotely. The remote collection of this information is obtained using a mobile application on a personal electronic device of the subject, such as a smartphone, tablet, or wearable electronic device. Non-limiting examples of the wearable electronic device include a fitness tracker (e.g., watch, wristband, ring, chest monitor), glucose monitor, heart rate monitor, sleep tracking device, and the like. The patient information may be self-reported by the subject, such as through manual entry into a mobile application on the subject's smartphone. The patient information may be automatically saved to a data store, such a cloud-based data store and retrieved from the data store by the patient-centric precision model.

### Example 6. Estimating Individual Parameter Estimates to Build the Patient-Centric Precision Model

Individual pharmacokinetic parameters were estimated using a combination of non-linear mixed effect models (NONMEM). The model employs 1st order absorption with one compartment and linear elimination with random effects on clearance (Cl), volume of distribution (central, [V1 Covariates consists of patient weight (on Cl, V1), albumin (ALB) on Cl, and c-reactive protein (CRP) on Cl. For the subject, ADA levels and weight were used to estimate the conditional distribution of the individual pharmacokinetic parameters. Following the estimation of the parameter estimates, the clearance before starting treatment is estimated using the weight associated with each of the covariates that forms the model. The equation is provided in Table 12.

### Example 7. Prophetic Example of Using a Combined PPF Scores Predict Therapeutic Regimen for an Inflammatory Bowel Disease Patient to Achieve Disease Remission

In this example, a biological sample is obtained from a patient with an inflammatory bowel disease (IBD) receiving IFX for treatment of the IBD. The biological sample is analyzed for levels of albumin, CRP, pANCA, PR3, and presence of genotypes at HLA DQA1*05, using the methods in Example 5. Patient wellbeing data is received, which includes the patient's weight, symptoms, and/or scores using standard disease activity indexes, such as CDAI and pMayo. Symptoms include remission, recurrence, or type, frequency, and/or severity of symptoms. Symptoms include diarrhea, fatigue, fever, rectal bleeding, joint pain, and rashes. The patient wellbeing data is obtained from the patient via a mobile App on the patient's smartphone or tablet, such as one illustrated in FIG. 8. The levels of these biomarkers and genotypes, and patient wellbeing data, are input into the patient-centric precision model.

The patient-centric precision model analyzes the presence of all four “poor prognosis factors” illustrated in FIG. 1, which include estimated clearance of IFX (PPF1) based on the patients weight and albumin levels as covariates. Inflammation was evaluated using CRP levels relative to a 3 mg/L cutoff in combination with patient wellbeing data. Total antigenic load was evaluated by measuring a level of PR3 relative to the level in a reference population of subjects without the IBD, and a presence or an absence of pANCA in a sample from the patient. In this example, the following data were input into the model: (a) CRP levels above 3 mg/L, pANCA presence detected, and PR3 above the 99^{th} percentile of the control individual without the IBD (PPF3=2); (b) presence of symptoms as indicated with a CDAI above 150 points, HBI above 5 points or pMayo above 2 points (PPF4=1); (c) carrier of risk allele at HLA DQA1*05 (AG) (PPF2=1); (d) body weight of 68 kilograms; and (e) albumin levels above 3.5 g/dL (PPF1=2).

In this example, the patient-centric precision model calculates a score of 6, which is indicative that the subject will not respond to infliximab.

Based on the score calculated above, a physician treating the IBD patient prescribes a therapeutic agent other than infliximab, such as a small molecule JAK kinase inhibitor, or S1P modulator.

### Example 8. Methods for Predicting Response to Anti-TNF Therapy

In this example, clearance and elimination of Infliximab (IFX) and adalimumab (ADA) containing volume was calculated using the population estimates derived while the patient is under treatment. The equation in Table 12 consists of 1) Clearance estimates at baseline, before starting treatment with albumin and weight as covariates, with or without C-reactive protein (CRP), 2) Clearance estimates at baseline, before starting treatment with albumin and weight as covariates, with or without CRP.

TABLE 12

| Infliximab (IFX) | Adalimumab (ADA) |
|---|---|
| Cl_base_ifx: = | Cl_base_ada: = |
| EXP(ln(0.294) − | EXP(−1.1394 − |
| 1.17*LN(ALB/4) + | 0.462*LN(ALB/4.12588) + |
| 0.614*LN(WT/70)) | 0.569*LN(WT/70.2995)) |
| CL_base_ifx_crp = | Cl_base_ada_crp: = |
| EXP(ln(0.294) − | EXP(−1.1394 − |
| 0.98*LN(ALB/4) + | 0.462*LN(ALB/4.12588) + |
| 0.614*LN(WT/70) + | 0.569*LN(WT/70.2995) + |
| 0.0399*LN(CRP/3) | 0.144*LN(CRP/1.95886)) |

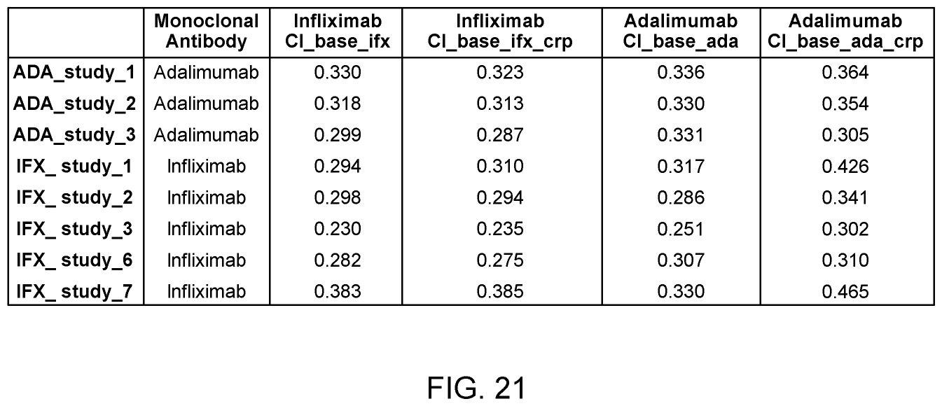

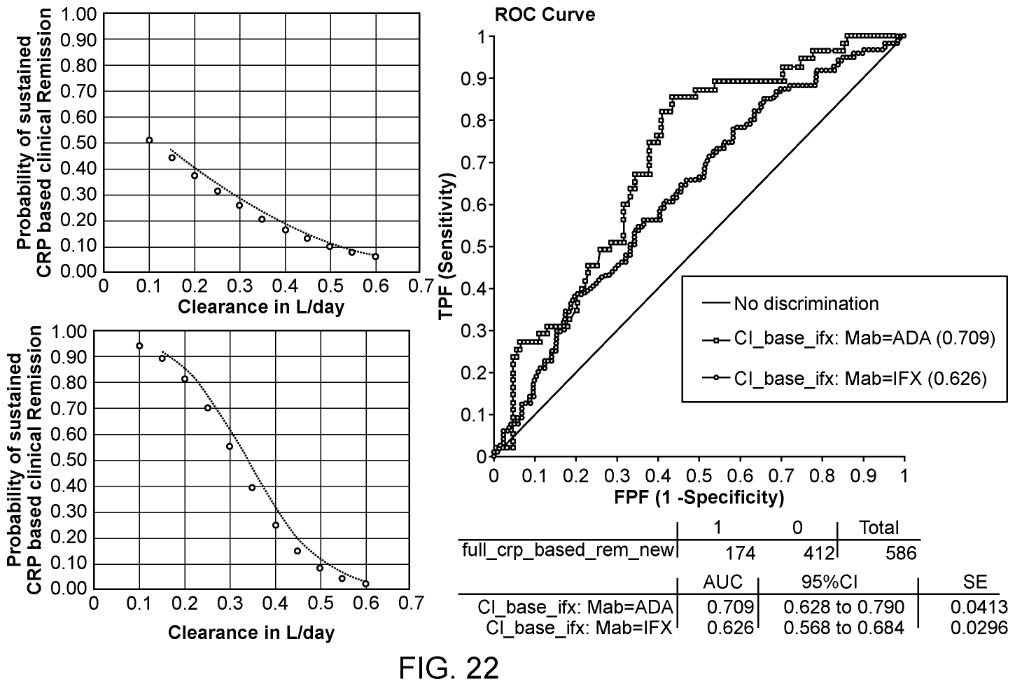

FIG. 21 provides clearance estimates for IFX (Cl_base_ifx) and ADA (Cl_base_ada) without and with CRP (Cl_base_ifx_crp and Cl_base_ada_crp), derived from the calculations above.

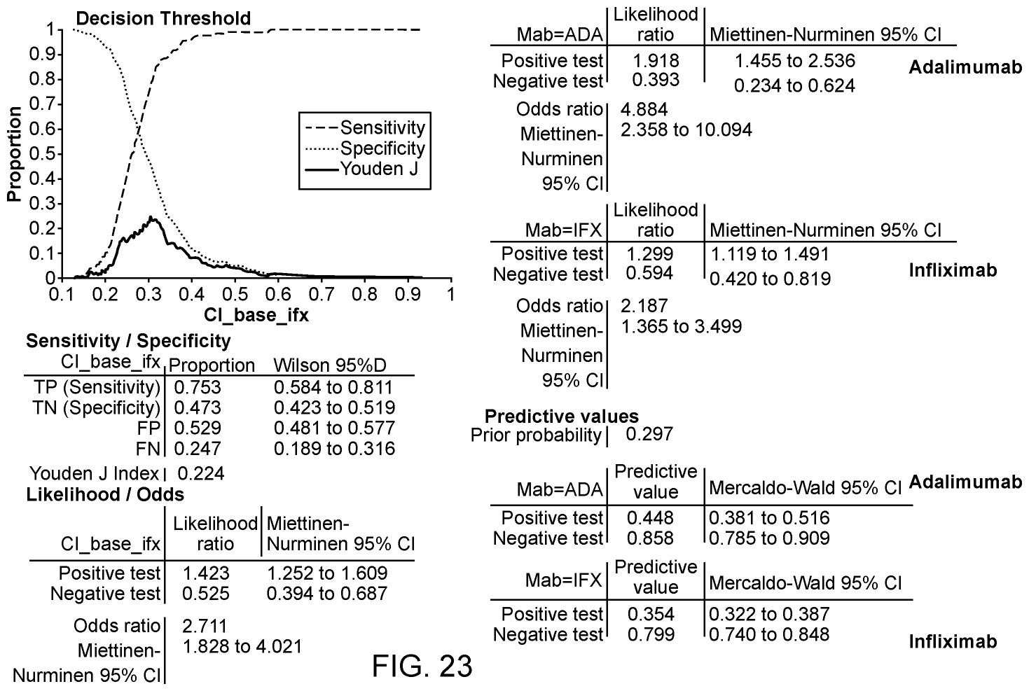

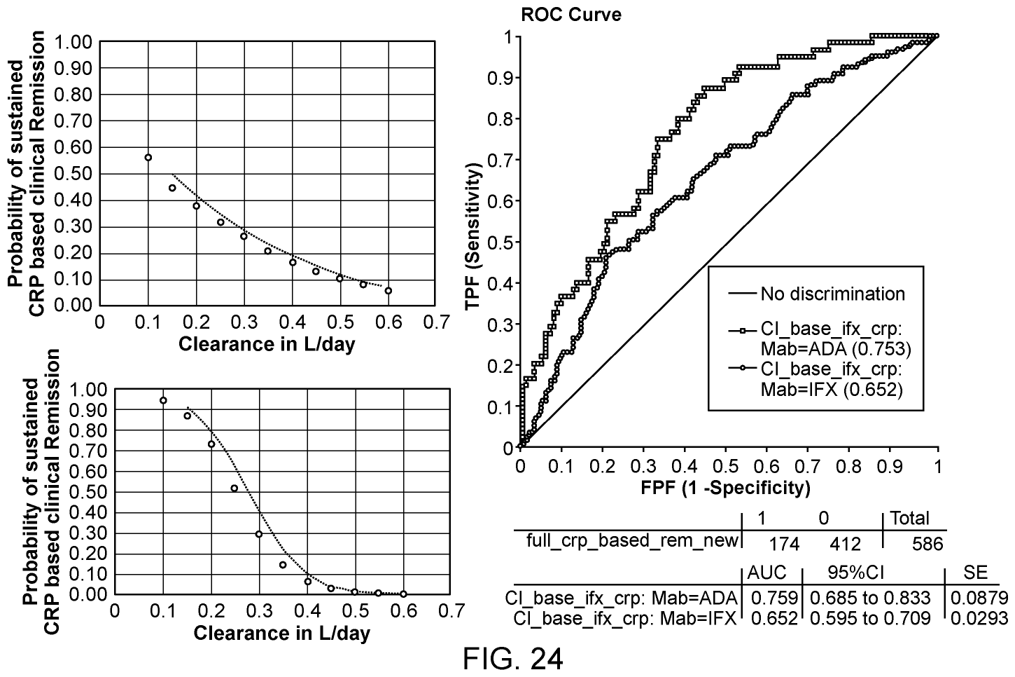

FIGS. 22-24 illustrate the performance of clearance pre-therapy in associating with outcome that consists of sustained CRP based clinical remission status over maintenance period of IFX treatment. Receiver operating characteristics curves are determined and the optimal cutoff associated with higher Youden score is determined. The probability for a sustained efficacy during treatment is calculated using logistic regression and is presented below with sensitivity, specificity, positive and negative predictive value.

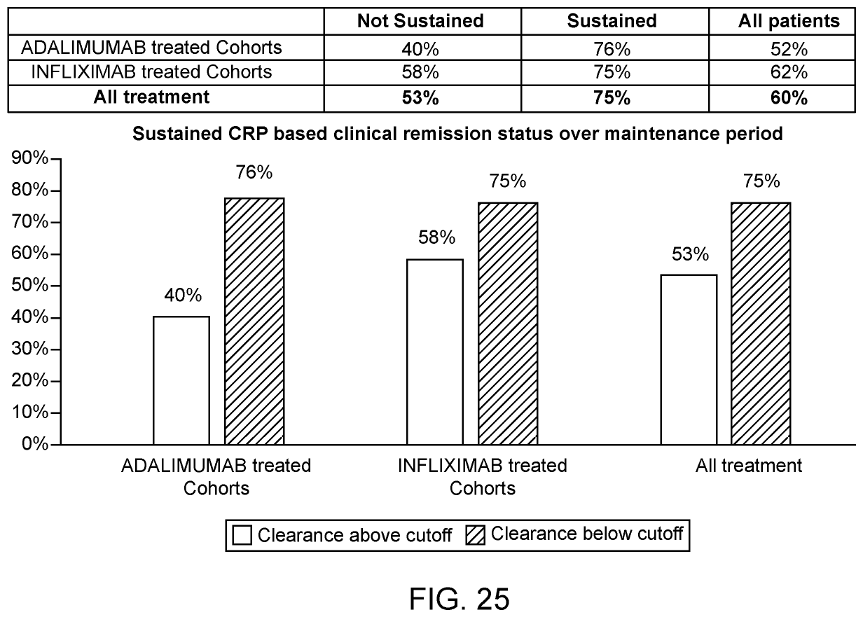

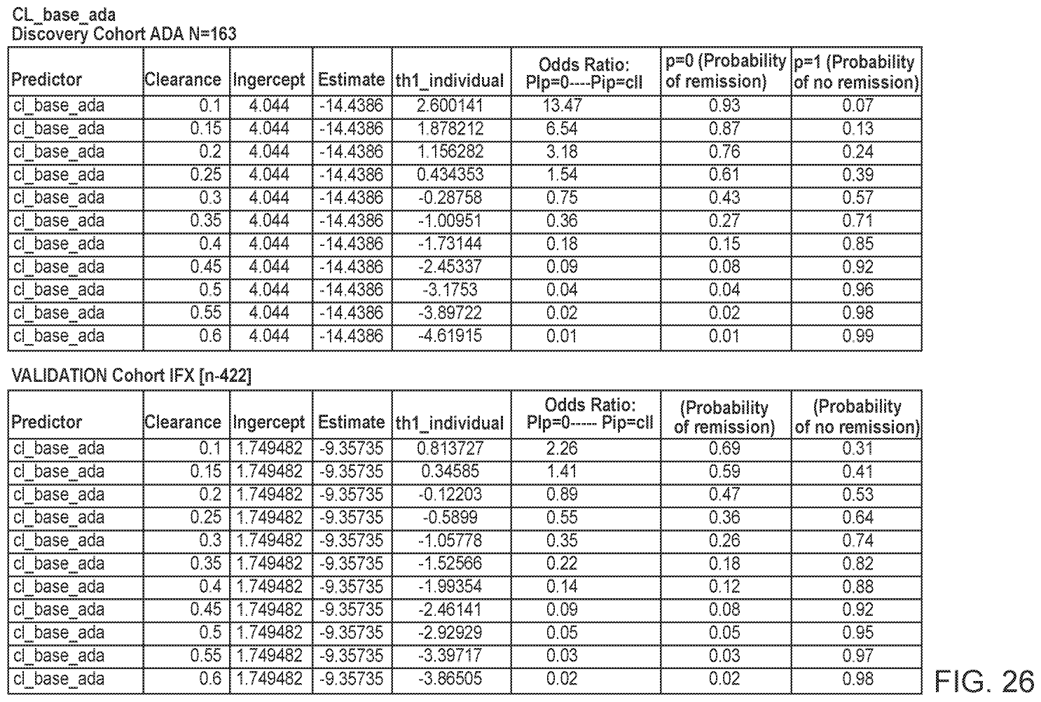

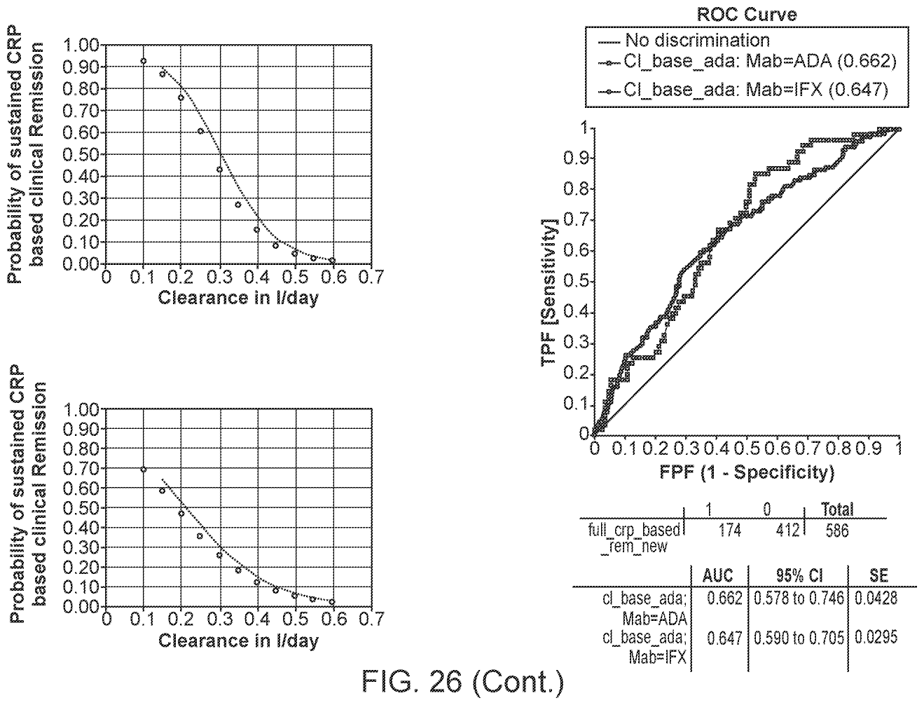

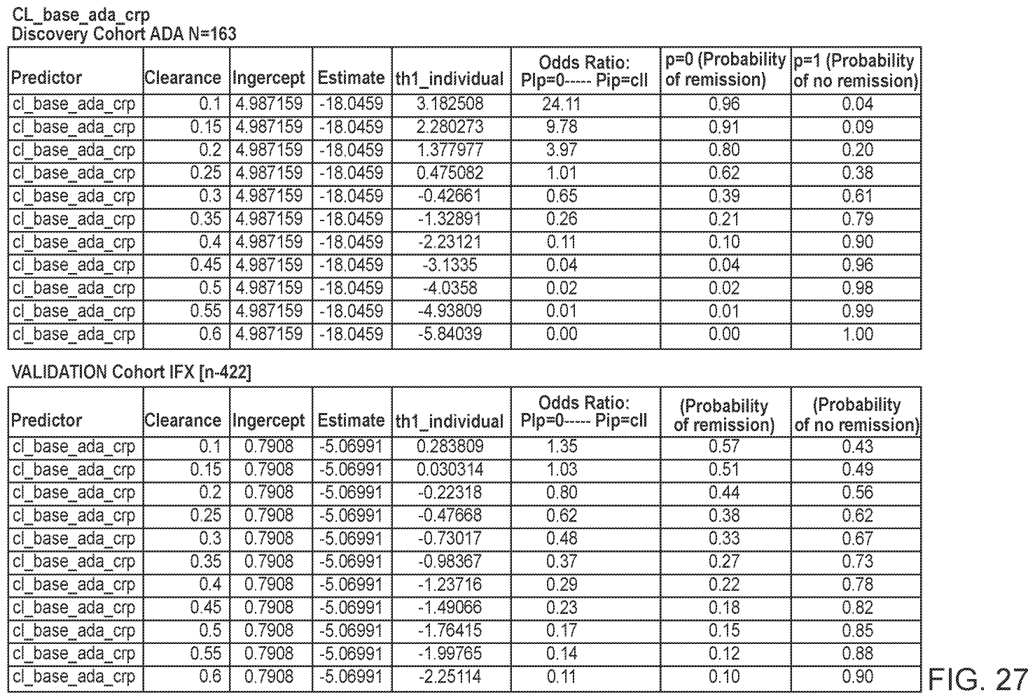

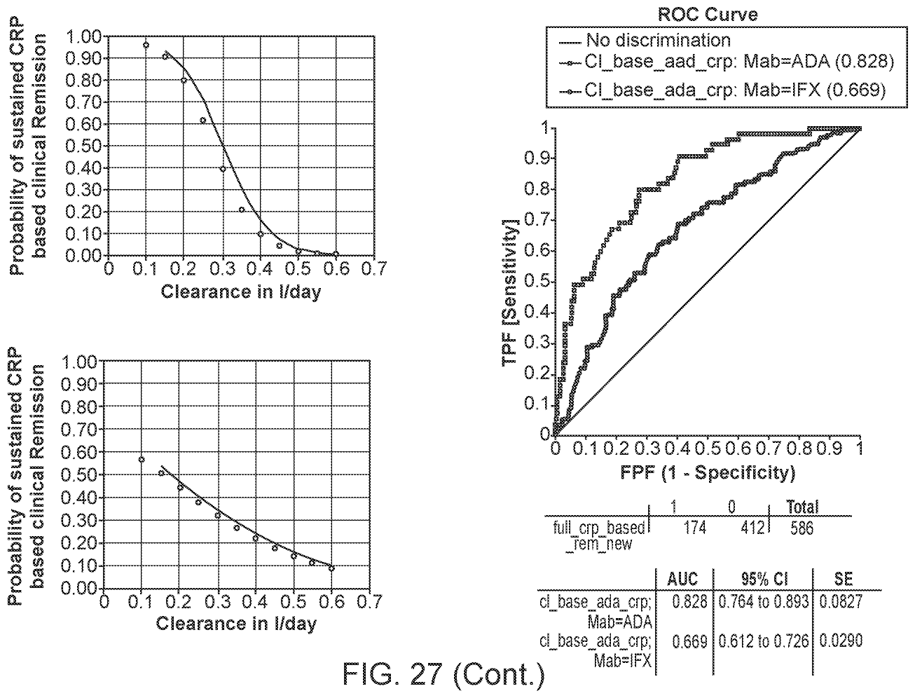

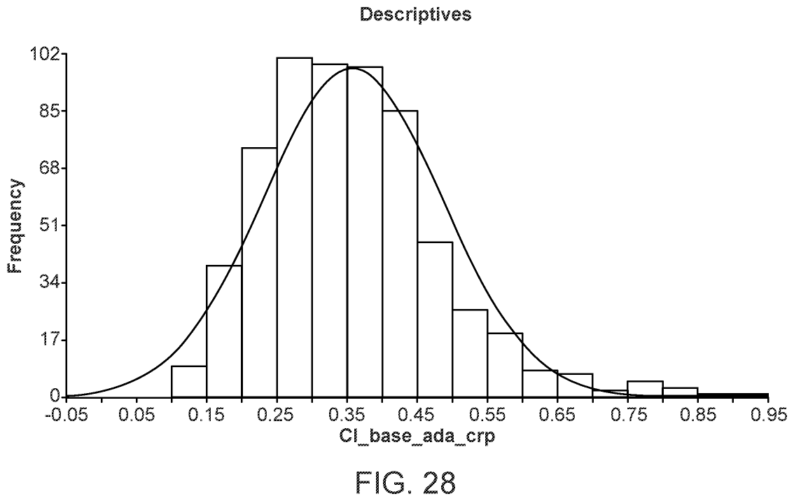

The probability of sustained versus not sustained CRP based clinical remission for IFX and ADA is presented in FIG. 25. FIG. 26 provides the clearance estimates for ADA (Cl_base_ada) without CRP. FIG. 27 provides the clearance estimates for ADA (Cl_base_ada_crp) with CRP. From the ADA (Cl_base_ada_crp) cohort analysis, Table 13 shows area under the curve (AUC) an confidential interval (95% CI) of statistical significance in bold. from patients from three separate studies involving treatment of inflammatory bowel disease (IBD) patients with ADA, and three separate studies involving treatment of IBD patients with IFX, using CRP as a covariate. A distribution of the predicted clearance estimates for those patients treated with ADA using CRP as a covariate is provided in FIG. 28.

TABLE 13

| Study | AUC | 95% CI | | SE |
|---|---|---|---|---|
| ADA_{—}study_{—}1 | 0.751 | 0.580 | to 0.922 | 0.0873 |
| ADA_{—}study_{—}2 | 0.752 | 0.620 | to 0.884 | 0.0674 |
| ADA_{—}study_{—}3 | 0.877 | 0.774 | to 0.980 | 0.0524 |
| IFX_study_1 | 0.659 | 0.488 | to 0.831 | 0.0875 |
| IFX_{—}study_{—}2 | 0.616 | 0.531 | to 0.702 | 0.0435 |
| IFX_study_3 | 0.527 | 0.352 | to 0.701 | 0.0892 |
| IFX_{—}study_{—}6 | 0.783 | 0.670 | to 0.897 | 0.0579 |
| IFX_{—}study_{—}7 | 0.720 | 0.516 | to 0.924 | 0.1042 |

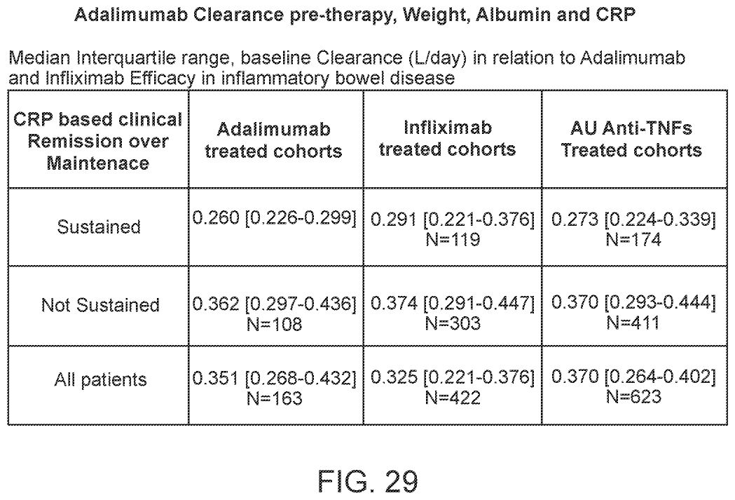

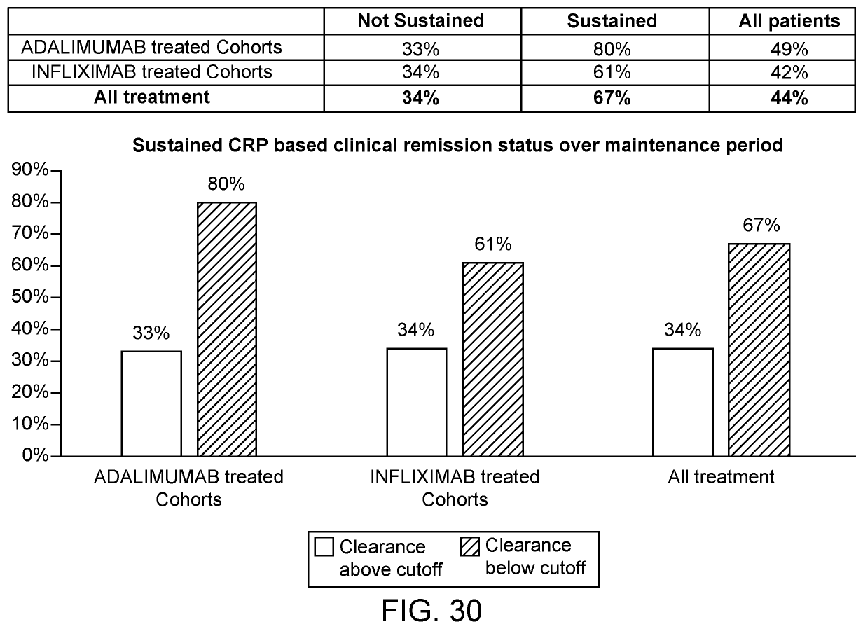

FIG. 29 shows median interquartile range, baseline clearance (L/day) in relation to ADA and IFX in IBD. Sustained clinical remission estimated using the patient-centric precision model is estimated for clearance of ADA of 0.260 or lower. Sustained clinical remission estimated using the patient-centric precision model is estimated for clearance of IFX of 0.291 or lower. FIG. 30 sows that in the ADA and IFX cohorts, fewer subjects with clearance above the relevant cutoff experienced remission over a maintenance period, than subjects with clearance below the relevant cutoff.

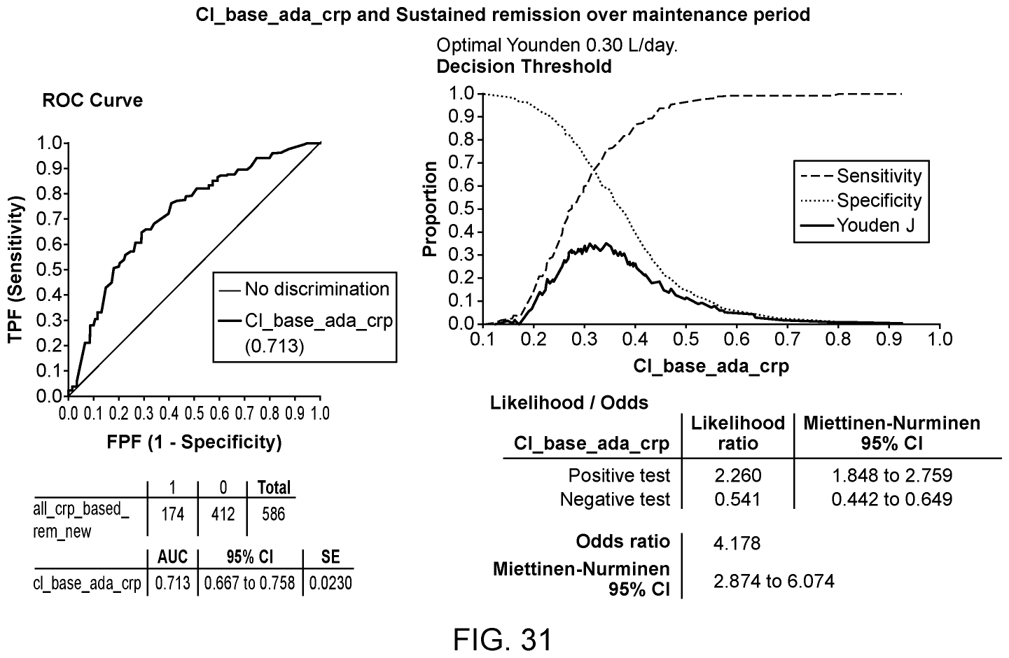

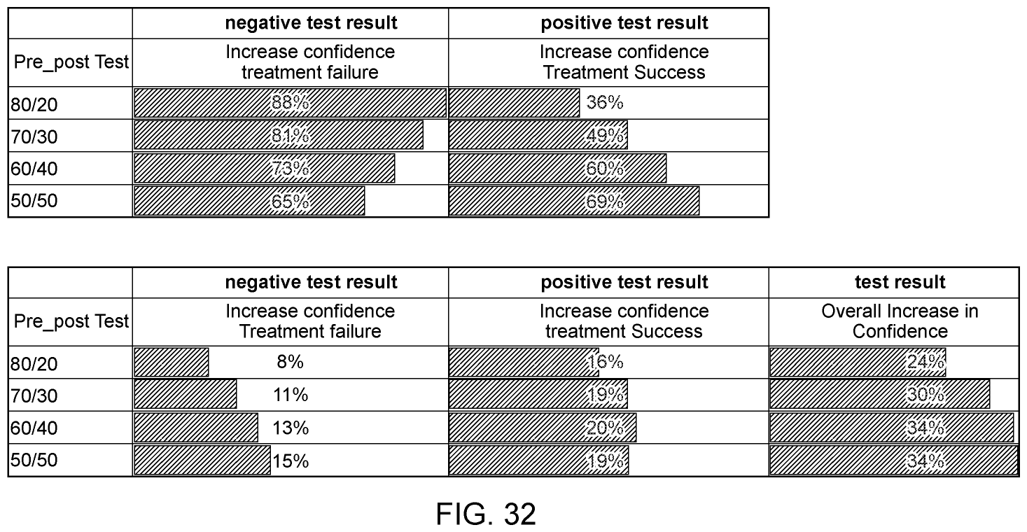

An optimal cutoff associated with higher Youden score is determined, as shown in FIG. 31. The positive and predictive value of the test using the optimal cutoff is calculated and provides the probability of treatment success or failure and achievement of lack thereof, respectively of sustained clinical remission in the presence of a negative test result at various pretest probability. An increase confidence in treatment failure or success is calculated for the monoclonal antibody. FIG. 32 shows a pre-post test illustrating increased confidence in predicting treatment failures with negative test results and treatment successes with positive test results.

Conclusion. The above example demonstrates that in IBD patient cohorts treated with ADA and IFX, that the patient-centric precision model is capable of predicting CRP-based clinical remission in subjects as a result of treatment with ADA and IFX with a high degree of confidence. While ADA and IFX were specifically tested, it is believed that the patient-centric precision model can predict CRP-based clinical remission for other monoclonal antibodies, such as for example, inhibitors of α₄β₇ integrin activity (e.g., Vedolizumab), inhibitor of interleukin 12 (IL-12), or interleukin 23 (IL-23) activity (e.g., Ustekinumab), inhibitors of TL1A, inhibitors of interleukin 6 (e.g., Tocilizumab) or other anti-TNF inhibitors, such as Etanercept, Golimumab, or Certolizumab.

### Example 9. Predicting Response to Anti-TNF Treatment Using PPF1

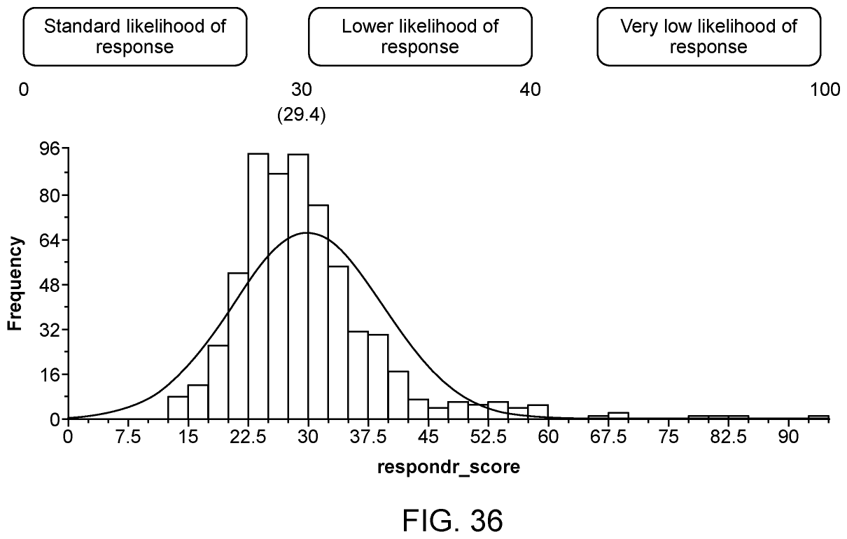

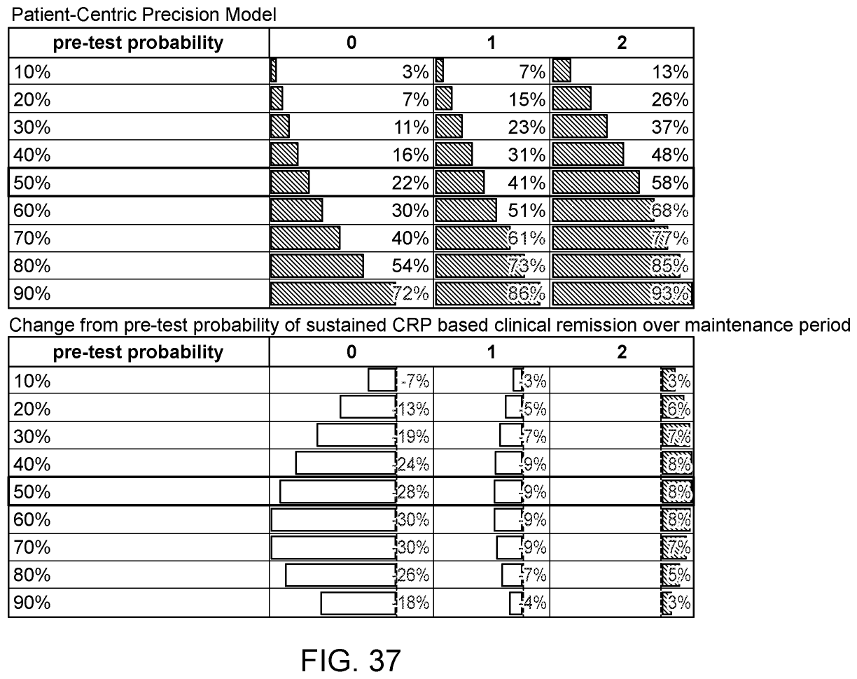

This example describes the method for estimate the likelihood of poor response to an anti-TNF agent based on PPF1 using the patient-centric precision model. A population of 586 patients started Infliximab or Adalimumab treatment. The PPF score is on a scale of 0 to 2 based on points ranging from 0 to 100 is calculated, where points below 30 (e.g., 29.4) points is associated with a “standard” response to an anti-TNF therapy (e.g., Infliximab or Adalimumab), points ranging from 30 to 40 points is associated with a low likelihood of response to the anti-TNF therapy, and points above 40 points which associates with a very low likelihood of response to the anti-TNF treatment. The performances were established in a population of 586 patients receiving TNF alpha blockers and consisting of Infliximab or adalimumab. The PPF score was calculated as follows before treatment (multiply by 100): CL_base_ifx: =EXP(ln(0.294)−1.17*LN(ALB/4)+0.614*LN(WT/70)). Table 14 provides the response status. FIG. 36 shows the distribution of scores associated with anti-TNF response, where the points are illustrated on the x axis and the frequency of subjects is shown on the y axis. FIG. 37 shows the performance of the patient-centric precision model in predicting anti-TNF response based on the PPF1 scores (0, 1, 2) calculated.

TABLE 14

| Variable | | Cl base ifx scale | | |
|---|---|---|---|---|
| Classification variable | | Full crp based rem new | | |
| Level | Positive* | Negative** | Likelihood ratio | 95% CI |
| 0 | 6 | 49 | 0.290 | 0.127 to 0.664 |
| 1 | 44 | 153 | 0.681 | 0.512 to 0.905 |
| 2 | 124 | 210 | 1.398 | 1.223 to 1.598 |
| Total | 174 | 412 | | |

*full_crp_based_rem_new = 1
**full_crp_based_rem_new = 0

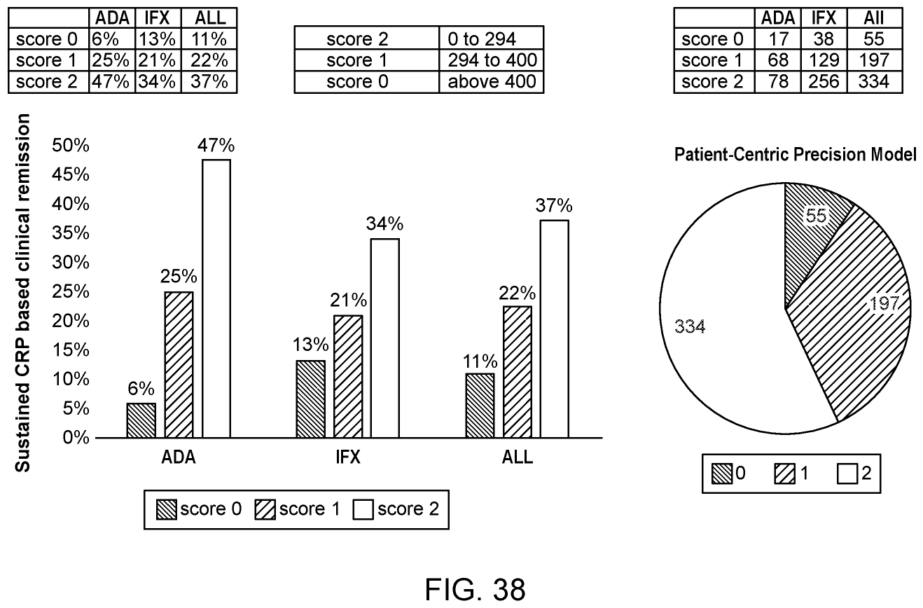

FIG. 37 shows that the change from pretest probability of response to an anti-TNF therapy during maintenance in subjects with a PPF1 score of 1 and 2 as determined using the patient-centric precision model is small. FIG. 38 shows that sustained clinical remission in achieved in IBD patients having a PPF1 score of 2 (left) and the distribution of scores using the patient-centric precision model (right).

### Example 10. Predicting Response to Anti-TNF Treatment Using Poor Prognosis Factor 3 (PPF3)

In this example, the PPF3 is composed of the presence of pANCA and Proteinase 3 (PR3) above a 99^{th} percentile of a reference population. Using the patient-centric precision model, it is estimated that patients with an immune-mediated inflammatory disease presenting with both pANCA and PR3 above the cutoffs corresponding to a PPF3 score of 2 are less likely to respond to treatment with a biologic drug, such as an anti-TNF therapy. Based on the results of the PPF3 score, a physician may prescribe a therapy other than a biologic drug to the patient to treat the immune-mediated inflammatory disease, such as a small molecule inhibitor of JAK or modulator of S1P.

### Example 11. Predicting Response to Anti-TNF Treatment Using PPF1 and PPF3

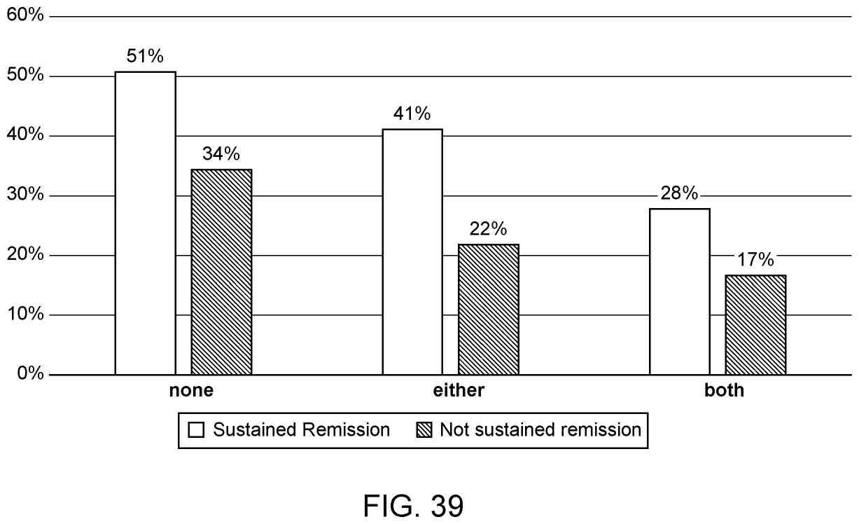

In this example, the PPF3 is composed of the presence of pANCA and Proteinase 3 (PR3) in the 99^{t}h percentile for a reference population that does not have the immune-mediated inflammatory disease. Using the patient-centric precision model, it is estimated that patients with an immune-mediated inflammatory disease presenting with a PPF3 score of 2 (as determined in Example 10) and PPF1 with score of 1 (as determined in Example 8) do not do well with treatment of the immune-mediated inflammatory disease with IFX, as shown in FIG. 39. The equation used for PPF1 is provided in Table 12. In this example, the physician may prescribe the patient a therapeutic agent other than a monoclonal antibody, such as a small molecule inhibitor of JAK or S1P, to treat the immune-mediated inflammatory disease.

### Example 12. Optimizing Treatment of an Immune-Mediated Inflammatory Disease with an Anti-Integrin α4β7 Therapy Using PPF1

This example describes the method for estimate the likelihood of poor response to an anti-integrin α4β7 therapy based on PPF1 using the patient-centric precision model. A population of patients is started on vedolizumab treatment. The PPF score is on a scale of 0 to 2 based on points ranging from 0 to 100 is calculated, where points below 30 (e.g., 29.4) points is associated with a “standard” response to an anti-integrin α4p7 therapy, points ranging from 30 to 40 points is associated with a low likelihood of response to the anti-integrin α4β7 therapy, and points above 40 points which associates with a very low likelihood of response to the anti-integrin α4β7 therapy treatment. The performances are established in the population of patients receiving vedolizumab. The PPF score is calculated as it is in Example 9. The patient-centric precision model recommends the following doses and inter-dose intervals for the patients based on the PPF score that is calculated. Without being bound by any particular theory, this structural model may be applied to any biologic drug, such as a monoclonal antibodies. Non-limiting examples of such monoclonal antibodies are provided below in Table 15 along with their recommended dose and inter-dose intervals.

TABLE 15

| | PPF1 = 0 or 1 | PPF1 = 2 |
|---|---|---|
| IFX | dose 10 mg/Kg 2, 6, 14 | Standard dose 5 mg/Kg 2, 6, |
| | weeks | 14 weeks |
| ADA | 160 mg at weeks 0, 1, 2, | Standard 160 mg at week 0 |
| | and 3 | and 80 mg at week 2 |
| VDZ | 300 mg 0, 2, 6, 10 weeks | Standard 300 mg 0, 2, 6, 14 |
| | | weeks |
| UST | Standard: 390 mg (55 Kg | Standard: 260 mg (55 Kg |
| | patient) | patient) |
| | Standard: 520 mg (55-85 Kg) | Standard: 390 mg (55-85 Kg) |
| | Standard: 650 mg (>85 Kg) | Standard: 520 mg (>85 Kg) |

What is claimed is:

1. A method for treating an immune-mediated inflammatory disease in a subject, said method comprising: administering a dose of an anti-tumor necrosis factor (TNF) therapy to the subject to treat the immune-mediated inflammatory disease, wherein the subject is selected based on a combined poor prognosis factor (PPF) score below 3 on a scale of 0 to 3 calculated using a statistical model, wherein the combined PPF score reflects a clearance estimate of the anti-TNF therapy and a presence of a genotype at a single nucleotide variant (SNV) comprising rs2097432 or a SNV in linkage disequilibrium therewith detected in a biological sample obtained from the subject.

2. A method for treating an immune-mediated inflammatory disease in a subject, said method comprising: administering a dose of an immune-mediated inflammatory disease therapy other than an anti-TNF therapy to the subject to treat the immune-mediated inflammatory disease, wherein the subject was selected based on combined poor prognosis factor (PPF) score of 3 on a scale of 0 to 3 calculated using a statistical model, wherein the combined PPF score reflects a clearance estimate of the anti-TNF therapy and a presence of a genotype at a single nucleotide variant (SNV) comprising rs2097432 or a SNV in linkage disequilibrium therewith detected in a biological sample obtained from the subject.

3. The method of claim 2, wherein the immune-mediated inflammatory disease therapy other than the anti-TNF therapy comprises a small molecule Janus Kinase (JAK) or a small molecule modulator of sphingosine 1-phosphate (S1P).

4. The method of claim 1, wherein the anti-TNF therapy comprises infliximab, adalimumab, etanercept, golimumab, or certolizumab.

5. The method of any one of claims 1-4, wherein the statistical model comprises one or more algorithms comprising a Naïve Bayes classifier algorithm, a non-linear mixed effects model (NLME), or a Metropolis Hastings algorithm, or any combination thereof.

6. The method of any one of claims 1-5, wherein the statistical model predicts a time to remission for the subject of the immune-mediated inflammatory disease.

7. The method of claim 6, wherein the statistical model predicts the time to remission for the subject by a method comprising:
(a) receiving data from a database, wherein the data is related to disease remission in individuals from a reference population having the immune-mediated inflammatory disease that have been treated with the anti-TNF therapy;
(b) establishing a first set of parameter estimates from the data;
(c) deriving a second set of parameter estimates for the statistical model based at least in part on the first set of parameter estimates;
(d) receiving subject specific data comprising: (i) the genotype at the SNV comprising rs2097432 or the SNV in linkage disequilibrium therewith; (ii) a weight or body mass index (BMI) of the subject; and (iii) a level of albumin measured in the biological sample;
(e) updating the model based at least in part on the subject specific data received in (d); and
(f) determining the time to remission for the subject, wherein the time to remission is reflected in a combined PPF score on the scale of 0 to 3.

8. The method of claim 7, wherein the subject specific data further comprises a level of c-reactive protein (CRP), wherein a CRP above 3 milligrams per liter (mg/L) is indicative of a presence of inflammation in the subject.

9. The method of claim 7, wherein the subject specific data further comprises a level of autoantibodies against the anti-TNF therapy, a level of the anti-TNF therapy, or a combination thereof.

10. The method of any one of claims 1-9, wherein the combined PPF score further reflects a wellbeing of the subject based on patient wellbeing data received by the structural model.

11. The method of claim 10, wherein the patient wellbeing data comprises a score on a disease activity index for the immune-mediated inflammatory disease.

12. The method of claim 11, wherein the disease activity index is Crohn's Disease Activity Index (CDAI) when the immune-mediated inflammatory disease is Crohn's disease.

13. The method of claim 11, wherein the disease activity index is a Mayo Clinical Score when the immune-mediated inflammatory disease is Ulcerative Colitis.

14. The method of claim of claim 10, wherein the patient wellbeing data comprises symptoms of the immune-mediated inflammatory disease, wherein the symptoms comprise diarrhea, abdominal pain, abdominal cramping, loss of appetite, weight loss, fatigue, fever, rectal bleeding, a skin rash, joint pain, stiffness, or swelling, decreased range of motion for joints, redness at joints, or any combination thereof.

15. The method of claim 10, wherein the patient wellbeing data comprises body mass index (BMI) or weight.

16. A method for treating an immune-mediated inflammatory disease in a subject, said method comprising:
(a) calculating a poor prognosis factor 1 (PPF1) score for the subject comprising:
(i) obtaining or having obtained a biological sample from the subject;
(ii) measuring a level of albumin in the biological sample;
(iii) calculating a rate of which an anti-tumor necrosis factor (TNF) therapy is estimated to be cleared from the subject using a first statistical model based, at least in part, on the level of the albumin measured in the biological sample and a weight of the subject; and
(iv) comparing the rate to a predetermined threshold for the anti-TNF therapy, to produce an estimated clearance score on a scale of 0 to 1, wherein 0 is indicative of a low rate of clearance below the predetermined threshold, and 1 is indicative of a high rate of clearance above the predetermined threshold;
(b) calculating a poor prognosis factor 2 (PPF 2) score for the subject, comprising:
(i) obtaining or having obtained the biological sample from the subject;
(ii) performing or having performed a genotyping assay on the biological sample to detect a genotype at single nucleotide variant (SNV) comprising rs2097432 or a SNV in linkage disequilibrium therewith; and
(iii) calculating a PPF2 score using a second statistical model, wherein the PPF2 score is on a scale of 0 to 2, wherein 0 is indicative of the genotype that is homozygous non-risk, 1 is indicative of the genotype that is heterozygous risk at rs2097432, and 2 comprises a presence of two genetic loci of said genotype;
(c) calculating a poor prognosis factor 4 (PPF 4) for the subject comprising:
(i) receiving wellbeing data from said subject comprising one or more symptoms related to the immune-mediated inflammatory disease;
(ii) calculating a wellbeing score with a fourth statistical model of 0 or 1 based on the wellbeing data received, wherein 0 is indicative of an improvement of at least one symptom of the one or more symptoms, and 1 is indicative of no improvement in the at least one symptom of the one or more symptoms; and
(d) calculating a combined PPF score comprising the PPF1 score, the PPF2 score, and the PPF4 score to predict a time to remission following administration of the anti-TNF therapy to the subject;
(e) if the time to remission predicted in (d) for the subject is above a pre-specified threshold, then administering a first dosage of the anti-TNF therapy to the subject to treat the immune-mediated inflammatory disease in the subject; and
(f) if the time to remission predicted in (d) for the subject is below the pre-specified threshold, then administering to the subject:
(i) a second dosage of the anti-TNF therapy, wherein the first dosage of the anti-TNF therapy is different than the second dosage of the anti-TNF therapy; or
(ii) a therapy other than the anti-TNF therapy.

17. The method of claim 16, further comprising calculating a poor prognosis factor 3 (PPF 3) score for the subject, comprising:
(i) obtaining or having obtained the biological sample from the subject;
(ii) performing or having performed an immunological assay on the biological sample to detect a level of a serological marker; and
(iii) calculating a PPF3 score using a third statistical model, wherein the PPF3 score is on a scale of 0 to 2, wherein 0 is indicative of a standard response to the anti-TNF therapy, 1 is indicative e of a low response to the anti-TNF therapy, and 2 is indicative of very low response to the anti-TNF therapy.

18. The method of claim 17, wherein the serological marker comprises perinuclear antineutrophil cytoplasmic antibodies (pANCA), proteinase 3 (PR3), or one or more antibodies against an integrin.

19. The method of any one of claims 16-18, wherein the predetermined threshold comprises about 0.2 to 0.4 L/day of the anti-TNF therapy.

20. The method of any one of claims 16-18, wherein the predetermined threshold is above about 0.3 L/day of the anti-TNF therapy.

21. The method of any one of claims 16-18, wherein the anti-TNF therapy is Infliximab.

22. The method of claim 21, wherein the pre-specified threshold is a percentage reduction in hazard remission above 78%.

23. The method of claim 22, wherein the first dose of the anti-TNF therapy comprises 5 milligrams per kilogram (mg/Kg) of Infliximab.

24. The method of any one of claims 16-20, wherein the pre-specified threshold is a percentage reduction in hazard remission comprising from 56% to 78%.

25. The method of claim 24, wherein the first dose of the anti-TNF therapy comprises 7.5 mg/Kg of Infliximab.

26. The method of any one of claims 16-20, wherein the pre-specified threshold is a percentage reduction in hazard remission that comprises from 34 to 56%.

27. The method of claim 26, wherein the first dose of the anti-TNF therapy comprises 10 mg/Kg of Infliximab.

28. The method of any one of claims 16-20, wherein the pre-specified threshold is a percentage reduction in hazard remission comprising 34% or less.

29. The method of claim 28, wherein the therapy other than the anti-TNF therapy comprises a small molecule Janus Kinase (JAK) or a small molecule modulator of sphingosine 1-phosphate (S1P).

30. The method of claim 29, wherein the small molecule inhibitor of JAK comprises Tofacitinib.

31. The method of claim 29, wherein the small molecule modulator of S1P comprises Ozanimod.

32. The method of any one of claims 16-31, wherein the first statistical model, the second statistical model, the third statistical model, or the fourth statistical model comprises one or more algorithms comprising a Naïve Bayes classifier algorithm, a non-linear mixed effects model (NLME), or a Metropolis Hastings algorithm, or any combination thereof.

33. The method of any one of claims 1-32, wherein the anti-TNF therapy comprises Infliximab, adalimumab, etanercept, golimumab, or certolizumab.

34. The method of any one of claims 1-32, wherein the immune-mediated inflammatory disease comprises an inflammatory bowel disease (IBD), rheumatoid arthritis (RA), cytokine release syndrome, multiple sclerosis (MS), ankylosing spondylitis (AS), lupus, plaque psoriasis, atopic dermatitis, gout, migraine, cancer, or a neoplasm.

35. The method of claim 34, wherein the IBD comprises Crohn's disease.

36. The method of claim 34, wherein the IBD comprises Ulcerative Colitis.

37. The method of any one of claims 1-36, wherein the biological sample obtained from the subject is blood serum.

38. The method of any one of claims 1-37, wherein the genotype at the SNV comprising rs2097432 comprises a “G.”

39. The method of claim 36, wherein the genotype at the SNV comprising s2097432 is homozygous.

40. The method of any one of claims 1-39, wherein the linkage disequilibrium is determined with an r² of at least about 0.84.

41. The method of any one of claims 1-4, wherein the subject is age 18 or older.

42. A computer-implemented system comprising a computing device comprising at least one processor, an operating system configured to perform executable instructions, a memory, and a computer program including instructions executable by the computing device to perform a method comprising:
(a) calculating a poor prognosis factor 1 (PPF1) score for the subject comprising:
(i) receiving a level of albumin measured in a biological sample obtained from a subject receiving anti-tumor necrosis factor (TNF) therapy for treatment of a immune-mediated inflammatory disease, and a weight of the subject;
(iii) calculating a rate of which the anti-TNF therapy is estimated to be cleared from the subject using a first statistical model based, at least in part, on the level of the albumin measured in the biological sample, and the weight of the subject; and
(iv) comparing the rate to a predetermined threshold for the anti-TNF therapy, to produce an estimated clearance score on a scale of 0 to 1, wherein 0 is indicative of a low rate of clearance below the predetermined threshold, and 1 is indicative of a high rate of clearance above the predetermined threshold;
(b) calculating a poor prognosis factor 2 (PPF 2) score for the subject comprising:
(i) receiving a genotype of the subject at a single nucleotide variant (SNV) comprising rs2097432 or a SNV in linkage disequilibrium therewith; and
(iii) calculating a PPF2 score using a second statistical model, wherein the PPF2 score is on a scale of 0 to 2, wherein 0 is indicative of the genotype that is homozygous non-risk, 1 is indicative of the genotype that is heterozygous risk at rs2097432, and 2 comprises a presence of two genetic loci of said genotype; and
(c) calculating a combined PPF score comprising the PPF1 score and the PPF2 score, to predict a time to remission following administration of the anti-TNF therapy to the subject.

43. The system of claim 42, wherein the method further comprises: (d) calculating a poor prognosis factor 3 (PPF 3) score for the subject, comprising:
(i) receiving a level of a serological marker detected in the biological sample obtained from the subject; and
(ii) calculating a PPF3 score using a third statistical model, wherein the PPF3 score is on a scale of 0 to 2, wherein 0 is indicative of a standard response to the anti-TNF therapy, 1 is indicative e of a low response to the anti-TNF therapy, and 2 is indicative of very low response to the anti-TNF therapy.

44. The system of claim 42 or 43, wherein the serological marker comprises perinuclear antineutrophil cytoplasmic antibodies (pANCA), proteinase 3 (PR3), or one or more antibodies against an integrin.

45. The system of any one of claims 42-44, wherein the method further comprises: (e) calculating a poor prognosis factor 4 (PPF 4) for the subject comprising:
(i) receiving wellbeing data from the subject comprising one or more symptoms related to the immune-mediated inflammatory disease;
(ii) calculating a wellbeing score with a fourth statistical model of 0 or 1 based on the wellbeing data received, wherein 0 is indicative of an improvement of at least one symptom of the one or more symptoms, and 1 is indicative of no improvement in the at least one symptom of the one or more symptoms, wherein the combined PPF score calculated in (c) further comprises the PPF score calculated in (d).

46. The system of any one of claims 42-45, wherein the anti-TNF therapy comprises Infliximab, Adalimumab, Etanercept, Golimumab, or Certolizumab.

47. The system of any one of claims 42-45, wherein the immune-mediated inflammatory disease comprises an inflammatory bowel disease (IBD), rheumatoid arthritis (RA), cytokine release syndrome, multiple sclerosis (MS), ankylosing spondylitis (AS), lupus, plaque psoriasis, atopic dermatitis, gout, migraine, cancer, or a neoplasm.

48. The system of claim 47, wherein the IBD comprises Crohn's disease.

49. The system of claim 47, wherein the IBD comprises Ulcerative Colitis.

50. The system of any one of claims 40-47, further comprising the biological sample, wherein the biological sample is blood serum.

51. The system of any one of claims 20-50, wherein the genotype at the SNV comprising rs2097432 comprises a “G.”

52. The system of claim 51, wherein the genotype at the SNV comprising s2097432 is homozygous.

53. The system of any one of claims 42-52, wherein the linkage disequilibrium is determined with an r² of at least about 0.84.

54. The system of any one of claims 42-53, wherein the subject is age 18 or older.

55. The system of any one of claims 42-54, wherein the predetermined threshold comprises about 0.2 to 0.4 L/day of the anti-TNF therapy.

56. The system of any one of claims 42-54, wherein the predetermined threshold is above about 0.3 L/day of the anti-TNF therapy.

57. The system of any one of claims 42-56, wherein the method further comprising determining a percentage reduction in hazard remission based on the time to remission.

58. The system of claim 57, wherein if the percentage reduction in hazard remission is above 34%, then the subject is indicated for treatment with Infliximab.

59. The system of claim 57, wherein if the percentage reduction in hazard remission is above 78%, then the subject is indicated for treatment with 5 mg/Kg of Infliximab.

60. The system of claim 57, wherein if the percentage reduction in hazard remission comprises from 56% to 78%, then the subject is indicated for treatment with 7.5 mg/Kg of Infliximab.

61. The system of claim 57, wherein if the percentage reduction in hazard remission comprises from 34% to 56%, then the subject is indicated for treatment with 10 mg/Kg of Infliximab.

62. The system of claim 57, wherein if the percentage reduction in hazard remission is 34% or below, then the subject is indicated for treatment with a therapy other than the anti-TNF therapy.

63. A non-transitory computer readable storage medium encoded with a computer program including instructions executable by one or more processors to statistic model for predicting disease remission in a subject with an immune-mediated inflammatory disease, the computer program configured to cause the one or more processors to perform a method comprising
(a) calculating a poor prognosis factor 1 (PPF1) score for the subject comprising:
(i) receiving a level of albumin measured in a biological sample obtained from a subject receiving anti-tumor necrosis factor (TNF) therapy for treatment of a immune-mediated inflammatory disease, and a weight of the subject;
(ii) calculating a rate of which the anti-TNF therapy is estimated to be cleared from the subject using a first statistical model based, at least in part, on the level of the albumin measured in the biological sample, and the weight of the subject; and
(iii) comparing the rate to a predetermined threshold for the anti-TNF therapy, to produce an estimated clearance score on a scale of 0 to 1, wherein 0 is indicative of a low rate of clearance below the predetermined threshold, and 1 is indicative of a high rate of clearance above the predetermined threshold;
(b) calculating a poor prognosis factor 2 (PPF 2) score for the subject comprising:
(i) receiving a genotype of the subject at a single nucleotide variant (SNV) comprising rs2097432 or a SNV in linkage disequilibrium therewith; and
(ii) calculating a PPF2 score using a second statistical model, wherein the PPF2 score is on a scale of 0 to 2, wherein 0 is indicative of the genotype that is homozygous non-risk, 1 is indicative of the genotype that is heterozygous risk at rs2097432, and 2 comprises a presence of two genetic loci of said genotype; and
(c) calculating a combined PPF score comprising the PPF1 score, the PPF2 score, and the PPF3 score to predict a time to remission following administration of the anti-TNF therapy to the subject.

64. The non-transitory computer-readable storage media of claim 63, wherein the method further comprises: (d) calculating a poor prognosis factor 3 (PPF 3) score for the subject, comprising:
(i) receiving a level of a serological marker detected in the biological sample obtained from the subject; and
(ii) calculating a PPF3 score using a third statistical model, wherein the PPF3 score is on a scale of 0 to 2, wherein 0 is indicative of a standard response to the anti-TNF therapy, 1 is indicative e of a low response to the anti-TNF therapy, and 2 is indicative of very low response to the anti-TNF therapy.

65. The non-transitory computer-readable storage media of claim 63, wherein the serological marker comprises perinuclear antineutrophil cytoplasmic antibodies (pANCA), proteinase 3 (PR3), or one or more antibodies against an integrin.

66. The non-transitory computer-readable storage media of any one of claims 63-65, wherein the method further comprises:
(e) calculating a poor prognosis factor 4 (PPF 4) for the subject comprising:
(i) receiving wellbeing data from the subject comprising one or more symptoms related to the immune-mediated inflammatory disease;
(ii) calculating a wellbeing score with a fourth statistical model of 0 or 1 based on the wellbeing data received, wherein 0 is indicative of an improvement of at least one symptom of the one or more symptoms, and 1 is indicative of no improvement in the at least one symptom of the one or more symptoms, wherein the combined PPF score calculated in (c) further comprises the PPF score calculated in (d).

67. The non-transitory computer-readable storage media of any one of claims 63-66, wherein the anti-TNF therapy comprises Infliximab, adalimumab, etanercept, golimumab, or certolizumab.

68. The non-transitory computer-readable storage media of any one of claims 63-67, wherein the immune-mediated inflammatory disease comprises an inflammatory bowel disease (IBD), rheumatoid arthritis (RA), cytokine release syndrome, multiple sclerosis (MS), ankylosing spondylitis (AS), lupus, plaque psoriasis, atopic dermatitis, gout, migraine, cancer, or a neoplasm.

69. The non-transitory computer-readable storage media of claim 68, wherein the IBD comprises Crohn's disease.

70. The non-transitory computer-readable storage media of claim 68, wherein the IBD comprises Ulcerative Colitis.

71. The non-transitory computer-readable storage media of any one of claims 63-70, wherein the biological sample is blood serum.

72. The non-transitory computer-readable storage media of any one of claims 63-71, wherein the genotype at the SNV comprising rs2097432 comprises a “G.”

73. The non-transitory computer-readable storage media of claim 72, wherein the genotype at the SNV comprising s2097432 is homozygous.

74. The non-transitory computer-readable storage media of any one of claims 63-73, wherein the linkage disequilibrium is determined with an r² of at least about 0.84.

75. The non-transitory computer-readable storage media of any one of claims 63-74, wherein the subject is age 18 or older.

76. The non-transitory computer-readable storage media of any one of claims 63-75, wherein the predetermined threshold comprises about 0.2 to 0.4 L/day of the anti-TNF therapy.

77. The non-transitory computer-readable storage media of any one of claims 63-75, wherein the predetermined threshold is above about 0.3 L/day of the anti-TNF therapy.

78. The non-transitory computer-readable storage media of any one of claims 63-77, wherein the method further comprising determining a percentage reduction in hazard remission based on the time to remission.

79. The non-transitory computer-readable storage media of claim 78, wherein if the percentage reduction in hazard remission is above 34%, then the subject is indicated for treatment with Infliximab.

80. The non-transitory computer-readable storage media of claim 78, wherein if the percentage reduction in hazard remission is above 78%, then the subject is indicated for treatment with 5 mg/Kg of Infliximab.

81. The non-transitory computer-readable storage media of claim 78, wherein if the percentage reduction in hazard remission comprises from 56% to 78%, then the subject is indicated for treatment with 7.5 mg/Kg of Infliximab.

82. The non-transitory computer-readable storage media of claim 78, wherein if the percentage reduction in hazard remission comprises from 34% to 56%, then the subject is indicated for treatment with 10 mg/Kg of Infliximab.

83. The non-transitory computer-readable storage media of claim 78, wherein if the percentage reduction in hazard remission is 34% or below, then the subject is indicated for treatment with a therapy other than the anti-TNF therapy.

84. A method of predicting whether an immune-mediated inflammatory disease in a subject will positively respond to a treatment with an anti-TNF therapy, the method comprising:
(a) calculating an estimated clearance rate at which an anti-tumor necrosis factor (TNF) therapy is cleared from the subject using a first statistical model based, at least in part, on a level of albumin measured in a biological sample obtained from the subject, and a weight of the subject;
(b) comparing the clearance rate calculated in (a) to a predetermined threshold, to produce poor prognosis factor 1 (PPF1) score on a scale of 0 to 1, wherein 0 is indicative of a low rate of clearance below the predetermined threshold, and 1 is indicative of a high rate of clearance above the predetermined threshold;
(c) calculating a poor prognosis factor 2 (PPF2) score on a scale of 0 to 2 with a second statistical model, based at least in part, on a presence of a genotype at a single nucleotide variant (SNV) comprising rs2097432 or a SNV in linkage disequilibrium therewith detected in the biological sample obtained from the subject, wherein 0 is indicative of the genotype that is homozygous non-risk, 1 is indicative of the genotype that is heterozygous risk at rs2097432, and 2 comprises a presence of two genetic loci of said genotype; and
(d) calculating a combined PPF score comprising the PPF1 score and the PPF2 score to predict whether the immune-mediated inflammatory disease in the subject will positively respond to the treatment with the anti-TNF therapy.

85. The method of claim 84, further comprising:
(e) calculating a poor prognosis factor 4 (PPF4) score on a scale of 0 to 1 with a third statistical model, based at least in part on patient wellbeing received from the subject, wherein a PPF4 score of 0 is indicative of an improvement of at least one symptom of the immune-mediated inflammatory disease, and a PPF4 score of 1 is indicative of no improvement in the at least one symptom, and wherein the combined PPF score calculated in (d) further comprises the PPF4 score.

86. The method of claim 84 or 85, further comprising: (f) calculating a poor prognosis factor 3 (PPF3) score on a scale of 0 to 2 with a fourth statistic model, wherein 0 is indicative of a standard response to the anti-TNF therapy, 1 is indicative e of a low response to the anti-TNF therapy, and 2 is indicative of very low response to the anti-TNF therapy.

87. The method of any one of claims 84-86, wherein the predetermined threshold is about 0.2 to 0.4 L/day.

88. The method of any one of claims 84-86, wherein the predetermined threshold is above 0.3 L/day.

89. The method of any one of claims 84-88, further comprising calculating a time to remission for the subject, wherein the time to remission is expressed as a percentage reduction in hazard remission.

90. The method of claim 89, wherein the percentage reduction in hazard remission is above 34% and the anti-TNF therapy comprises Infliximab.

91. The method of claim 89, further comprising administering to the subject 5 mg/Kg of Infliximab, provided the percentage reduction in hazard remission is above 78%.

92. The method of claim 89, further comprising administering to the subject 7.5 mg/Kg of Infliximab, provided the percentage reduction in hazard remission comprises from 56% to 78%.

93. The method of claim 89, further comprising administering to the subject 10 mg/Kg of Infliximab, provided the percentage reduction in hazard remission comprises 34% to 56%.

94. The method of claim 89, further comprising administering to the subject a therapy other than the anti-TNF therapy, provided the percentage reduction in hazard remission is 34% or below.

95. The method of claim 4, wherein the therapy other than the anti-TNF therapy comprises a small molecule inhibitor of Janus Kinase (JAK) or a small molecule modulator of sphingosine 1-phosphate (S1P).

96. The method of claim 95, wherein the small molecule inhibitor of Janus Kinase (JAK) comprises Tofacitinib.

97. The method of any one of claims 84-96, wherein the first statistical model and the second statistical model comprises one or more algorithms comprising a Naïve Bayes classifier algorithm, a non-linear mixed effects model (NLME), or a Metropolis Hastings algorithm, or any combination thereof.

98. The method of any one of claims 84-97, wherein the anti-TNF therapy comprises Infliximab, adalimumab, etanercept, golimumab, or certolizumab.

99. The method of any one of claims 84-98, wherein the immune-mediated inflammatory disease comprises an inflammatory bowel disease (IBD), rheumatoid arthritis (RA), cytokine release syndrome, multiple sclerosis (MS), ankylosing spondylitis (AS), lupus, plaque psoriasis, atopic dermatitis, gout, migraine, cancer, or a neoplasm.

100. The method of claim 99, wherein the IBD comprises Crohn's disease.

101. The method of claim 99, wherein the IBD comprises Ulcerative Colitis.

102. The method of any one of claims 84-101, wherein the biological sample obtained from the subject is blood serum.

103. The method of any one of claims 84-102, wherein the genotype at the SNV comprising rs2097432 comprises a “G.”

104. The method of claim 103, wherein the genotype at the SNV comprising s2097432 is homozygous.

105. The method of any one of claims 84-104, wherein the linkage disequilibrium is determined with an r² of at least about 0.84.

106. The method of any one of claims 84-105, wherein the subject is age 18 or older.

107. The method of any one of claims 84-106, wherein the patient wellbeing data comprises a score on a disease activity index for the immune-mediated inflammatory disease.

108. The method of claim 107, wherein the disease activity score index comprises Crohn's Disease Activity Index (CDAI) or a Mayo Clinical Score.
